(19) United States
(12) Patent Application Publication
(10) Pub. No.: US 2024/0208942 A1
(43) Pub. Date: Jun. 27, 2024

(54) METAP-2 INHIBITORS, PHARMACEUTICAL COMPOSITIONS AND THERAPEUTIC METHODS THEREOF

(71) Applicant: CANWELL BIOTECH LIMITED, Hong Kong (CN)

(72) Inventors: RONGLIANG LOU, Cheshire, CT (US); NINGHUI YU, Wellesley, MA (US)

(21) Appl. No.: 18/280,548

(22) PCT Filed: Mar. 14, 2022
(86) PCT No.: PCT/CN2022/080544

§ 371 Date: Sep. 6, 2023

Related U.S. Application Data
(60) Provisional application No. 63/167,078, filed on Mar. 28, 2021.

(51) Int. Cl. C07D 403/04; A61K 31/404; A61K 31/407; A61K 31/428; A61K 31/437; A61K 31/4985; C07D 417/04; C07D 471/04; C07D 487/04; C07D 495/04

(52) U.S. Cl. CPC C07D 403/04; A61K 31/404; A61K 31/407; A61K 31/428; A61K 31/437; A61K 31/4985; C07D 417/04; C07D 471/04; C07D 487/04; C07D 495/04

(57) ABSTRACT

The invention provides novel inhibitors of methionine aminopeptidase 2 (MetAP-2) and pharmaceutical compositions thereof, and their use in and methods of treatment, reduction or prevention of certain diseases or conditions associated with MetAP-2 (e.g., cancer, obesity, rheumatoid arthritis and psoriasis).

### PRIORITY CLAIMS AND RELATED APPLICATIONS

This application claims the benefit of priority to U.S. Provisional Application No. 63/167,078, filed Mar. 28, 2021, the entire content of which is incorporated herein by reference for all purposes.

### TECHNICAL FIELD OF THE INVENTION

The invention generally relates to compounds and therapeutic uses thereof. More particularly, the invention provides novel compounds, e.g., inhibitors of methionine aminopeptidase 2 (MetAP-2) and pharmaceutical compositions thereof, and their use in and methods of treatment, reduction or prevention of certain diseases or conditions associated with MetAP-2 (e.g., cancer, obesity, rheumatoid arthritis and psoriasis).

### BACKGROUND OF THE INVENTION

Methionine aminopeptidases (MetAPs) are intracellular metalloproteins responsible for the removal of the initiator NH₂-terminal methionine from newly synthesized proteins, thereby facilitating their intracellular translocation from the ribosome. They are necessary for different protein cotranslational and/or posttranslational modifications, such as NH₂— terminal myristoylation or acetylation.

Two types of MetAP enzymes have been generally found, MetAP-1 and MetAP-2, which have similar three-dimensional structures despite low homology in their sequences (Biochim Biophys Acta 2000; 1477:157-167). In recent years, studies have suggested that MetAP-1 could play an important role in the G2/M phase of the cell cycle and that it may serve as a promising target for the development of new anticancer agents (Proc Natl Acad Sci USA 2006; 103:18148-18153). MetAP-2 has attracted more attention than MetAP-1 due to its identification as a target molecule of the anti-angiogenic compounds. Different studies suggest that MetAP-2 may play a central role in endothelial cell proliferation, and higher concentrations of MetAP-2 have been detected in tumors compared with normal tissue (Am J Pathol 2001; 159:721-731. Lab Invest 2002; 82:893-901). MetAP-2 inhibition is able to induce G1 cell cycle arrest and cytostasis of tumor cells in vitro, and to reduce tumor growth in vivo (Biochemistry 2003; 42:5035-5042. Proc Natl Acad Sci USA 2000; 97:6427-6432). Moreover, it has been demonstrated the physical interaction between MetAP-2 and the metastasis-associated protein S100A4, and the effects of this association on endothelial cell growth and tumor metastasis (J Biol Chem 2002; 277:26396-26402). MetAP-2 inhibitors could be useful in the treatment of a broad type of cancers by their potential ability to inhibit tumor angiogenesis and metastasis. Furthermore, MetAP-2 inhibition also has potential in the treatment of cell proliferative diseases, including rheumatoid arthritis and psoriasis.

Various pre-clinical and clinical studies have been reported that involve the use of MetAP-2 inhibitors in treating various diseases and conditions. Nevertheless, the therapeutics and methods currently available for the management of diseases or conditions associated with MetAP-2 remain inadequate. There is an urgent and ongoing need for novel and improved therapeutics to effectively treat such diseases and conditions.

### SUMMARY OF THE INVENTION

The invention is based in part on the unexpected discovery of novel MetAP-2 inhibitors, methods of their synthesis, and pharmaceutical compositions as well as methods thereof for treating, preventing or reducing various diseases or conditions associated with MetAP-2, for example, cancer, obesity, diabetes rheumatoid arthritis and psoriasis.

In one aspect, the invention generally relates to a compound having the structural formula of (I), or a pharmaceutically acceptable form or an isotope derivative thereof:

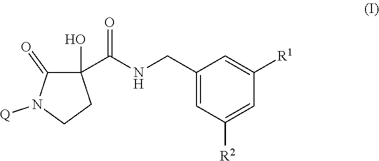

wherein
each of R¹ and R² is independently selected from F, Cl, Br, and I;
Q is (a) an unsubstituted or substituted 5- or 6-membered aromatic ring, or (b) an unsubstituted or substituted 5- or 6-membered aromatic ring fused with a second unsubstituted or substituted 5- or 6-membered ring.

In another aspect, the invention generally relates to a pharmaceutical composition comprising a compound disclosed herein and a pharmaceutically acceptable excipient, carrier, or diluent.

In another aspect, the invention generally relates to a pharmaceutical composition comprising an amount of a compound having the structural formula (I), or a pharmaceutically acceptable form or an isotope derivative thereof:

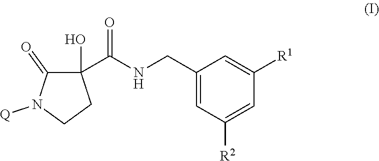

wherein

each of R¹ and R² is independently selected from F, Cl, Br, and I; Q is
(a) an unsubstituted or substituted 5- or 6-membered aromatic ring, or
(b) an unsubstituted or substituted 5- or 6-membered aromatic ring fused with a second unsubstituted or substituted 5- or 6-membered ring.

In yet another aspect, the invention generally relates to a unit dosage form comprising a pharmaceutical composition of the invention.

In yet another aspect, the invention generally relates to a method for treating, reducing, or preventing a disease or condition, comprising administering to a subject in need thereof a pharmaceutical composition comprising a compound having the structural formula of (I), or a pharmaceutically acceptable form or an isotope derivative thereof:

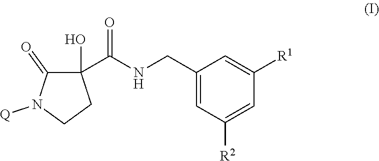

wherein

each of R^{t} and R² is independently selected from F, Cl, Br, and I; Q is
(a) an unsubstituted or substituted 5- or 6-membered aromatic ring, or
(b) an unsubstituted or substituted 5- or 6-membered aromatic ring fused with a second unsubstituted or substituted 5- or 6-membered ring,
   effective to treat, prevent, or reduce one or more of cancer, obesity, diabetes rheumatoid arthritis, psoriasis, or a related disease or condition thereof, in a mammal, including a human, and a pharmaceutically acceptable excipient, carrier, or diluent.

In yet another aspect, the invention generally relates to use of a compound of the invention for treating or reducing a disease or condition.

In yet another aspect, the invention generally relates to use of a compound of the invention, and a pharmaceutically acceptable excipient, carrier, or diluent, in preparation of a medicament for treating or reducing a disease or condition.

### Definitions

Unless defined otherwise, all technical and scientific terms used herein have the same meaning as commonly understood by one of ordinary skill in the art to which this invention belongs. General principles of organic chemistry, as well as specific functional moieties and reactivity, are described in “Organic Chemistry”, Thomas Sorrell, University Science Books, Sausalito: 1999, and “March's Advanced Organic Chemistry”, 5^{th} Ed.: Smith, M. B. and March, J., John Wiley & Sons, New York: 2001, the entire contents of which are hereby incorporated by reference.

Certain compounds of the present invention may exist in particular geometric or stereoisomeric forms. The present invention contemplates all such compounds, including cis- and trans-isomers, R- and S-enantiomers, diastereomers, (D)-isomers, (L)-isomers, the racemic mixtures thereof, and other mixtures thereof, as falling within the scope of the invention. Additional asymmetric carbon atoms may be present in a substituent such as an alkyl group. All such isomers, as well as mixtures thereof, are intended to be included in this invention.

Isomeric mixtures containing any of a variety of isomer ratios may be utilized in accordance with the present invention. For example, where only two isomers are combined, mixtures containing 50:50, 60:40, 70:30, 80:20, 90:10, 95:5, 96:4, 97:3, 98:2, 99:1, or 100:0 isomer ratios are contemplated by the present invention. Those of ordinary skill in the art will readily appreciate that analogous ratios are contemplated for more complex isomer mixtures.

If, for instance, a particular enantiomer of a compound of the present invention is desired, it may be prepared by asymmetric synthesis, or by derivation with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts are formed with an appropriate optically-active acid or base, followed by resolution of the diastereomers thus formed by fractional crystallization or chromatographic methods well known in the art, and subsequent recovery of the pure enantiomers.

Solvates and polymorphs of the compounds of the invention are also contemplated herein. Solvates of the compounds of the present invention include, for example, hydrates.

Definitions of specific functional groups and chemical terms are described in more detail below. When a range of values is listed, it is intended to encompass each value and sub-range within the range. For example, “C₁₋₆ alkyl” is intended to encompass, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

As used herein, the term “alkyl” refers to a straight, branched or cyclic hydrocarbon radical consisting solely of carbon and hydrogen atoms, containing no unsaturation, having from one to ten carbon atoms (e.g., C₁₋₁₀₀ alkyl). Whenever it appears herein, a numerical range such as “1 to 10” refers to each integer in the given range; e.g., “1 to 10 carbon atoms” means that the alkyl group can consist of 1 carbon atom, 2 carbon atoms, 3 carbon atoms, etc., up to and including 10 carbon atoms, although the present definition also covers the occurrence of the term “alkyl” where no numerical range is designated. In some embodiments, “alkyl” can be a C₁₋₆ alkyl group. In some embodiments, alkyl groups have 1 to 10, 1 to 8, 1 to 6, or 1 to 3 carbon atoms.

Representative saturated straight chain alkyls include, but are not limited to, -methyl,-ethyl, -n-propyl, -n-butyl, -n-pentyl, and -n-hexyl; while saturated branched alkyls include, but are not limited to, -isopropyl, -sec-butyl, -isobutyl, -tert-butyl, -isopentyl, 2-methylbutyl, 3-methylbutyl, 2-methylpentyl, 3-methylpentyl, 4-methylpentyl, 2-methylhexyl, 3-methylhexyl, 4-methylhexyl, 5-methylhexyl, 2,3-dimethylbutyl, and the like. The alkyl is attached to the parent molecule by a single bond.

Unless stated otherwise in the specification, an alkyl group is optionally substituted by one or more of substituents which independently include: acyl, alkyl, alkenyl, alkynyl, alkoxy, alkylaryl, cycloalkyl, aralkyl, aryl, aryloxy, amino, amido, amidino, imino, azide, carbonate, carbamate, carbonyl, heteroalkyl, heteroaryl, heteroarylalkyl, heterocycloalkyl, hydroxy, cyano, halo (F, Cl, Br, I), haloalkoxy, haloalkyl, ester, ether, mercapto, thio, alkylthio, arylthio, thiocarbonyl, nitro, oxo, phosphate, phosphonate, phosphinate, silyl, sulfinyl, sulfonyl, sulfonamidyl, sulfoxyl, sulfonate, urea, —Si(R^{a})₃, —OR^{a}, —SR^{a}, —OC(O)—R^{a}, —N(R^{a})₂, —C(O)Rₐ, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, —N(R^{a})C(NR^{a})N(R^{a})₂, —N(R^{a})S(O)ₜN(R^{a})₂ (where t is 1 or 2), —P(═O)(R^{a})(R^{a}), or —O—P(═O)(OR^{a})₂ where each R^{a} is independently hydrogen, alkyl, haloalkyl, carbocyclyl, carbocyclylalkyl, aryl, aralkyl, heterocycloalkyl, heterocycloalkylalkyl, heteroaryl or heteroarylalkyl, and each of these moieties can be optionally substituted as defined herein. In a non-limiting embodiment, a substituted alkyl can be selected from fluoromethyl, difluoromethyl, trifluoromethyl, 2-fluoroethyl, 3-fluoropropyl, hydroxymethyl, 2-hydroxyethyl, 3-hydroxypropyl, benzyl, and phenethyl.

As used herein, the terms “aliphatic” or “aliphatic group” means a straight-chain (i.e., unbranched) or branched, substituted or unsubstituted hydrocarbon chain that is completely saturated or that contains one or more units of unsaturation, or a monocyclic hydrocarbon or bicyclic hydrocarbon that is completely saturated or that contains one or more units of unsaturation, but which is not aromatic, that has a single point of attachment to the rest of the molecule. Unless otherwise specified, aliphatic groups contain 1-6 aliphatic carbon atoms. In some embodiments, aliphatic groups contain 1-5 aliphatic carbon atoms. In other embodiments, aliphatic groups contain 1-4 aliphatic carbon atoms. In still other embodiments, aliphatic groups contain 1-3 aliphatic carbon atoms, and in yet other embodiments, aliphatic groups contain 1-2 aliphatic carbon atoms. In some embodiments, “cycloaliphatic” refers to a monocyclic C₃-C₆ hydrocarbon that is completely saturated or that contains one or more units of unsaturation, but which is not aromatic, that has a single point of attachment to the rest of the molecule. Exemplary aliphatic groups are linear or branched, substituted or unsubstituted C₁-C₈ alkyl, C₂-C₈ alkenyl, C₂-C₈ alkynyl groups and hybrids thereof such as (cycloalkyl)alkyl, (cycloalkenyl)alkyl or (cycloalkyl)alkenyl.

As used herein, the terms “aromatic” or “aryl” refer to a radical with 6 to 14 ring atoms (e.g., C₆₋₁₄ aromatic or C₆₋₁₄ aryl) that has at least one ring having a conjugated pi electron system which is carbocyclic (e.g., phenyl, fluorenyl, and naphthyl). In some embodiments, the aryl is a C₆₋₁₀ aryl group. For example, bivalent radicals formed from substituted benzene derivatives and having the free valences at ring atoms are named as substituted phenylene radicals. In other embodiments, bivalent radicals derived from univalent polycyclic hydrocarbon radicals whose names end in “-yl” by removal of one hydrogen atom from the carbon atom with the free valence are named by adding “-idene” to the name of the corresponding univalent radical, e.g., a naphthyl group with two points of attachment is termed naphthylidene. Whenever it appears herein, a numerical range such as “6 to 14 aryl” refers to each integer in the given range; e.g., “6 to 14 ring atoms” means that the aryl group can consist of 6 ring atoms, 7 ring atoms, etc., up to and including 14 ring atoms. The term includes monocyclic or fused-ring polycyclic (i.e., rings which share adjacent pairs of ring atoms) groups. Polycyclic aryl groups include bicycles, tricycles, tetracycles, and the like. In a multi-ring group, only one ring is required to be aromatic, so groups such as indanyl are encompassed by the aryl definition. Non-limiting examples of aryl groups include phenyl, phenalenyl, naphthalenyl, tetrahydronaphthyl, phenanthrenyl, anthracenyl, fluorenyl, indolyl, indanyl, and the like. Unless stated otherwise in the specification, an aryl moiety can be optionally substituted by one or more substituents which independently include: acyl, alkyl, alkenyl, alkynyl, alkoxy, alkylaryl, cycloalkyl, aralkyl, aryl, aryloxy, amino, amido, amidino, imino, azide, carbonate, carbamate, carbonyl, heteroalkyl, heteroaryl, heteroarylalkyl, heterocycloalkyl, hydroxy, cyano, halo, haloalkoxy, haloalkyl, ester, ether, mercapto, thio, alkylthio, arylthio, thiocarbonyl, nitro, oxo, phosphate, phosphonate, phosphinate, silyl, sulfinyl, sulfonyl, sulfonamidyl, sulfoxyl, sulfonate, urea, —Si(R^{a})₃, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, —N(R^{a})C(NR^{a})N(R^{a})₂, —N(R^{a})S(O)ₜN(R^{a})₂ (where t is 1 or 2), —P(═O)(R^{a})(R^{a}) or —O—P(═O)(OR^{a})₂ where each R^{a} is independently hydrogen, alkyl, haloalkyl, carbocyclyl, carbocyclylalkyl, aryl, aralkyl, heterocycloalkyl, heterocycloalkylalkyl, heteroaryl or heteroarylalkyl, and each of these moieties can be optionally substituted as defined herein.

As used herein, the terms “cycloalkyl” and “carbocyclyl” each refers to a monocyclic or polycyclic radical that contains only carbon and hydrogen, and can be saturated or partially unsaturated. Partially unsaturated cycloalkyl groups can be termed “cycloalkenyl” if the carbocycle contains at least one double bond, or “cycloalkynyl” if the carbocycle contains at least one triple bond. Cycloalkyl groups include groups having from 3 to 13 ring atoms (i.e., C₃₁3 cycloalkyl). Whenever it appears herein, a numerical range such as “3 to 10” refers to each integer in the given range; e.g., “3 to 13 carbon atoms” means that the cycloalkyl group can consist of 3 carbon atoms, 4 carbon atoms, 5 carbon atoms, etc., up to and including 13 carbon atoms. The term “cycloalkyl” also includes bridged and spiro-fused cyclic structures containing no heteroatoms. The term also includes monocyclic or fused-ring polycyclic (i.e., rings which share adjacent pairs of ring atoms) groups. Polycyclic aryl groups include bicycles, tricycles, tetracycles, and the like. In some embodiments, “cycloalkyl” can be a C₃₋₈ cycloalkyl radical. In some embodiments, “cycloalkyl” can be a C₃₋₅ cycloalkyl radical. Illustrative examples of cycloalkyl groups include, but are not limited to the following moieties: C₃₋₆ carbocyclyl groups include, without limitation, cyclopropyl (C₃), cyclobutyl (C₄), cyclopentyl (C₅), cyclopentenyl (C₅), cyclohexyl (C₆), cyclohexenyl (C₆), cyclohexadienyl (C₆) and the like. Examples of C₃₋₇ carbocyclyl groups include norbornyl (C₇). Examples of C₃₋₈ carbocyclyl groups include the aforementioned C₃₋₇ carbocyclyl groups as well as cycloheptyl (C₇), cycloheptadienyl (C₇), cycloheptatrienyl (C₇), cyclooctyl (C₈), bicyclo[2.2.1]heptanyl, bicyclo[2.2.2]octanyl, and the like. Examples of C₃₋₁₃ carbocyclyl groups include the aforementioned C₃₋₈ carbocyclyl groups as well as octahydro-1H indenyl, decahydronaphthalenyl, spiro[4.5]decanyl and the like. Unless stated otherwise in the specification, a cycloalkyl group can be optionally substituted by one or more substituents which independently include: acyl, alkyl, alkenyl, alkynyl, alkoxy, alkylaryl, cycloalkyl, aralkyl, aryl, aryloxy, amino, amido, amidino, imino, azide, carbonate, carbamate, carbonyl, heteroalkyl, heteroaryl, heteroarylalkyl, heterocycloalkyl, hydroxy, cyano, halo, haloalkoxy, haloalkyl, ester, ether, mercapto, thio, alkylthio, arylthio, thiocarbonyl, nitro, oxo, phosphate, phosphonate, phosphinate, silyl, sulfinyl, sulfonyl, sulfonamidyl, sulfoxyl, sulfonate, urea, —Si(R^{a})₃, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, —N(R^{a})C(NR^{a})N(R^{a})₂, —N(R^{a})S(O)ₜN(R^{a})₂ (where t is 1 or 2), —P(═O)(R^{a})(R^{a}), or —O—P(═O)(OR^{a})₂ where each R^{a} is independently hydrogen, alkyl, haloalkyl, carbocyclyl, carbocyclylalkyl, aryl, aralkyl, heterocycloalkyl, heterocycloalkylalkyl, heteroaryl or heteroarylalkyl, and each of these moieties can be optionally substituted as defined herein. The terms “cycloalkenyl” and “cycloalkynyl” mirror the above description of “cycloalkyl” wherein the prefix “alk” is replaced with “alken” or “alkyn” respectively, and the parent “alkenyl” or “alkynyl” terms are as described herein. For example, a cycloalkenyl group can have 3 to 13 ring atoms, such as 5 to 8 ring atoms. In some embodiments, a cycloalkynyl group can have 5 to 13 ring atoms.

As used herein, the term “inhibit” refers to any measurable reduction of biological activity. Thus, as used herein, “inhibit” or “inhibition” may be referred to as a percentage of a normal level of activity.

As used herein, the term “effective amount” or “therapeutically effective amount” of an active agent refers to an amount sufficient to elicit the desired biological response. The effective amount, when administered in a proper dosing regimen, is sufficient to reduce or ameliorate the severity, duration or progression of the disorder being treated, prevent the advancement of the disorder being treated, cause the regression of the disorder being treated, or enhance or improve the prophylactic or therapeutic effect(s) of another therapy. As will be appreciated by those of ordinary skill in this art, the effective amount of a compound of the invention may vary depending on such factors as the desired biological endpoint, the pharmacokinetics of the compound, the disease being treated, the mode of administration, and the patient. An effective amount can be readily determined by a skilled physician, e.g., by first administering a low dose of the pharmacological agent(s) and then incrementally increasing the dose until the desired therapeutic effect is achieved with minimal or no undesirable side effects.

As used herein, the terms “treatment” or “treating” a disease or disorder refers to a method of reducing, delaying or ameliorating such a condition before or after it has occurred. Treatment may be directed at one or more effects or symptoms of a disease and/or the underlying pathology. The treatment can be any reduction and can be, but is not limited to, the complete ablation of the disease or the symptoms of the disease. As compared with an equivalent untreated control, such reduction or degree of prevention is at least 5%, 10%, 20%, 40%, 50%, 60%, 80%, 90%, 95%, or 100% as measured by any standard technique.

As used herein, the terms “prevent”, “preventing”, or “prevention” refer to a method for precluding, delaying, averting, or stopping the onset, incidence, severity, or recurrence of a disease or condition. For example, a method is considered to be a prevention if there is a reduction or delay in onset, incidence, severity, or recurrence of a disease or condition or one or more symptoms thereof in a subject susceptible to the disease or condition as compared to a subject not receiving the method. The disclosed method is also considered to be a prevention if there is a reduction or delay in onset, incidence, severity, or recurrence of osteoporosis or one or more symptoms of a disease or condition in a subject susceptible to the disease or condition after receiving the method as compared to the subject's progression prior to receiving treatment. Thus, the reduction or delay in onset, incidence, severity, or recurrence of osteoporosis can be about a 5, 10, 20, 30, 40, 50, 60, 70, 80, 90, 100%, or any amount of reduction in between.

As used herein, a “pharmaceutically acceptable form” of a disclosed compound includes, but is not limited to, pharmaceutically acceptable salts, esters, hydrates, solvates, polymorphs, isomers, prodrugs, and isotopically labeled derivatives thereof. In one embodiment, a “pharmaceutically acceptable form” includes, but is not limited to, pharmaceutically acceptable salts, esters, prodrugs and isotopically labeled derivatives thereof. In some embodiments, a “pharmaceutically acceptable form” includes, but is not limited to, pharmaceutically acceptable isomers and stereoisomers, prodrugs and isotopically labeled derivatives thereof.

In certain embodiments, the pharmaceutically acceptable form is a pharmaceutically acceptable salt. As used herein, the term “pharmaceutically acceptable salt” refers to those salts which are, within the scope of sound medical judgment, suitable for use in contact with the tissues of subjects without undue toxicity, irritation, allergic response and the like, and are commensurate with a reasonable benefit/risk ratio. Pharmaceutically acceptable salts are well known in the art. For example, Berge et al. describes pharmaceutically acceptable salts in detail in J. Pharmaceutical Sciences (1977) 66:1-19. Pharmaceutically acceptable salts of the compounds provided herein include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchioric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, besylate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. In some embodiments, organic acids from which salts can be derived include, for example, acetic acid, propionic acid, glycolic acid, pyruvic acid, oxalic acid, lactic acid, trifluoracetic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like.

The salts can be prepared in situ during the isolation and purification of the disclosed compounds, or separately, such as by reacting the free base or free acid of a parent compound with a suitable base or acid, respectively. Pharmaceutically acceptable salts derived from appropriate bases include alkali metal, alkaline earth metal, ammonium and N+(C₁₋₄alkyl)₄ salts. Representative alkali or alkaline earth metal salts include sodium, lithium, potassium, calcium, magnesium, iron, zinc, copper, manganese, aluminum, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate and aryl sulfonate. Organic bases from which salts can be derived include, for example, primary, secondary, and tertiary amines, substituted amines, including naturally occurring substituted amines, cyclic amines, basic ion exchange resins, and the like, such as isopropylamine, trimethylamine, diethylamine, triethylamine, tripropylamine, and ethanolamine. In some embodiments, the pharmaceutically acceptable base addition salt can be chosen from ammonium, potassium, sodium, calcium, and magnesium salts.

In certain embodiments, the pharmaceutically acceptable form is a “solvate” (e.g., a hydrate). As used herein, the term “solvate” refers to compounds that further include a stoichiometric or non-stoichiometric amount of solvent bound by non-covalent intermolecular forces. The solvate can be of a disclosed compound or a pharmaceutically acceptable salt thereof. Where the solvent is water, the solvate is a “hydrate”. Pharmaceutically acceptable solvates and hydrates are complexes that, for example, can include 1 to about 100, or 1 to about 10, or 1 to about 2, about 3 or about 4, solvent or water molecules. It will be understood that the term “compound” as used herein encompasses the compound and solvates of the compound, as well as mixtures thereof.

In certain embodiments, the pharmaceutically acceptable form is a prodrug. As used herein, the term “prodrug” (or “pro-drug”) refers to compounds that are transformed in vivo to yield a disclosed compound or a pharmaceutically acceptable form of the compound. A prodrug can be inactive when administered to a subject, but is converted in vivo to an active compound, for example, by hydrolysis (e.g., hydrolysis in blood). In certain cases, a prodrug has improved physical and/or delivery properties over the parent compound. Prodrugs can increase the bioavailability of the compound when administered to a subject (e.g., by permitting enhanced absorption into the blood following oral administration) or which enhance delivery to a biological compartment of interest (e.g., the brain or lymphatic system) relative to the parent compound. Exemplary prodrugs include derivatives of a disclosed compound with enhanced aqueous solubility or active transport through the gut membrane, relative to the parent compound.

The prodrug compound often offers advantages of solubility, tissue compatibility or delayed release in a mammalian organism (see, e.g., Bundgard, H., Design of Prodrugs (1985), pp. 7-9, 21-24 (Elsevier, Amsterdam). A discussion of prodrugs is provided in Higuchi, T., et al., “Pro-drugs as Novel Delivery Systems,” A.C.S. Symposium Series, Vol. 14, and in Bioreversible Carriers in Drug Design, ed. Edward B. Roche, American Pharmaceutical Association and Pergamon Press, 1987, both of which are incorporated in full by reference herein. Exemplary advantages of a prodrug can include, but are not limited to, its physical properties, such as enhanced water solubility for parenteral administration at physiological pH compared to the parent compound, or it can enhance absorption from the digestive tract, or it can enhance drug stability for long-term storage.

Prodrugs commonly known in the art include well-known acid derivatives, such as, for example, esters prepared by reaction of the parent acids with a suitable alcohol, amides prepared by reaction of the parent acid compound with an amine, basic groups reacted to form an acylated base derivative, etc. Of course, other prodrug derivatives may be combined with other features disclosed herein to enhance bioavailability. As such, those of skill in the art will appreciate that certain of the presently disclosed compounds having free amino, arnido, hydroxy or carboxylic groups can be converted into prodrugs. Prodrugs include compounds having an amino acid residue, or a polypeptide chain of two or more (e.g., two, three or four) amino acid residues which are covalently joined through peptide bonds to free amino, hydroxy or carboxylic acid groups of the presently disclosed compounds. The amino acid residues include the 20 naturally occurring amino acids commonly designated by three letter symbols and also include 4-hydroxyproline, hydroxylysine, demosine, isodemosine, 3-methylhistidine, norvalin, beta-alanine, gamma—aminobutyric acid, citrulline homocysteine, homoserine, ornithine and methionine sulfone. Prodrugs also include compounds having a carbonate, carbamate, amide or alkyl ester moiety covalently bonded to any of the above substituents disclosed herein.

As used herein, the term “pharmaceutically acceptable” excipient, carrier, or diluent refers to a pharmaceutically acceptable material, composition or vehicle, such as a liquid or solid filler, diluent, excipient, solvent or encapsulating material, involved in carrying or transporting the subject pharmaceutical agent from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations. Wetting agents, emulsifiers and lubricants, such as sodium lauryl sulfate, magnesium stearate, and polyethylene oxide-polypropylene oxide copolymer as well as coloring agents, release agents, coating agents, sweetening, flavoring and perfuming agents, preservatives and antioxidants can also be present in the compositions.

As used herein, the terms “isolated” or “purified” refer to a material that is substantially or essentially free from components that normally accompany it in its native state. Purity and homogeneity are typically determined using analytical chemistry techniques such as polyacrylamide gel electrophoresis or high-performance liquid chromatography.

As used herein, the term “subject” refers to any animal (e.g., a mammal), including, but not limited to humans, non-human primates, rodents, and the like, which is to be the recipient of a particular treatment. Typically, the terms “subject” and “patient” are used interchangeably herein in reference to a human subject.

As used herein, the term “low dosage” refers to at least 5% less (e.g., at least 10%, 20%, 50%, 80%, 90%, or even 95%) than the lowest standard recommended dosage of a particular compound formulated for a given route of administration for treatment of any human disease or condition. For example, a low dosage of an agent that is formulated for administration by inhalation will differ from a low dosage of the same agent formulated for oral administration.

As used herein, the term “high dosage” is meant at least 5% (e.g., at least 10%, 20%, 50%, 100%, 200%, or even 300%) more than the highest standard recommended dosage of a particular compound for treatment of any human disease or condition.

Isotopically-labeled compounds are also within the scope of the present disclosure. As used herein, an “isotopically-labeled compound” or “isotope derivative” refers to a presently disclosed compound including pharmaceutical salts and prodrugs thereof, each as described herein, in which one or more atoms are replaced by an atom having an atomic mass or mass number different from the atomic mass or mass number usually found in nature. Examples of isotopes that can be incorporated into compounds presently disclosed include isotopes of hydrogen, carbon, nitrogen, oxygen, phosphorous, fluorine and chlorine, such as ²H, ³H, ¹³C, ¹⁴C, ¹⁵N, ¹⁸O, ¹⁷O ³¹P, ³²P, ³⁵S, ¹⁸F, and ³⁶C₁, respectively.

By isotopically-labeling the presently disclosed compounds, the compounds may be useful in drug and/or substrate tissue distribution assays. Tritiated (3H) and carbon-14 (¹⁴C) labeled compounds are particularly preferred for their ease of preparation and detectability. Further, substitution with heavier isotopes such as deuterium (2H) can afford certain therapeutic advantages resulting from greater metabolic stability, for example increased in vivo half-life or reduced dosage requirements and, hence, may be preferred in some circumstances. Isotopically labeled compounds presently disclosed, including pharmaceutical salts, esters, and prodrugs thereof, can be prepared by any means known in the art. Benefits may also be obtained from replacement of normally abundant ¹²C with ¹³C. (See, WO 2007/005643, WO 2007/005644, WO 2007/016361, and WO 2007/016431.)

For example, deuterium (2H) can be incorporated into a compound disclosed herein for the purpose in order to manipulate the oxidative metabolism of the compound by way of the primary kinetic isotope effect. The primary kinetic isotope effect is a change of the rate for a chemical reaction that results from exchange of isotopic nuclei, which in turn is caused by the change in ground state energies necessary for covalent bond formation after this isotopic exchange. Exchange of a heavier isotope usually results in a lowering of the ground state energy for a chemical bond and thus causes a reduction in the rate in rate-limiting bond breakage. If the bond breakage occurs in or in the vicinity of a saddle-point region along the coordinate of a multi-product reaction, the product distribution ratios can be altered substantially. For explanation: if deuterium is bonded to a carbon atom at a non-exchangeable position, rate differences of k_{M}/k_{D}=2-7 are typical. If this rate difference is successfully applied to a compound disclosed herein that is susceptible to oxidation, the profile of this compound in vivo can be drastically modified and result in improved pharmacokinetic properties.

When discovering and developing therapeutic agents, the person skilled in the art is able to optimize pharmacokinetic parameters while retaining desirable in vitro properties. It is reasonable to assume that many compounds with poor pharmacokinetic profiles are susceptible to oxidative metabolism. In vitro liver microsomal assays currently available provide valuable information on the course of oxidative metabolism of this type, which in turn permits the rational design of deuterated compounds of those disclosed herein with improved stability through resistance to such oxidative metabolism. Significant improvements in the pharmacokinetic profiles of compounds disclosed herein are thereby obtained, and can be expressed quantitatively in terms of increases in the in vivo half-life (t/2), concen-tra-tion at maximum therapeutic effect (Cₘₐₓ), area under the dose response curve (AUC), and F; and in terms of reduced clearance, dose and materials costs.

The following is intended to illustrate the above: a compound which has multiple potential sites of attack for oxidative metabolism, for example benzylic hydrogen atoms and hydrogen atoms bonded to a nitrogen atom, is prepared as a series of analogues in which various combinations of hydrogen atoms are replaced by deuterium atoms, so that some, most or all of these hydrogen atoms have been replaced by deuterium atoms. Half-life determinations enable favorable and accurate determination of the extent of the extent to which the improvement in resistance to oxidative metabolism has improved. In this way, it is determined that the half-life of the parent compound can be extended by up to 100% as the result of deuterium-hydrogen exchange of this type.

Deuterium-hydrogen exchange in a compound disclosed herein can also be used to achieve a favorable modification of the metabolite spectrum of the starting compound in order to diminish or eliminate undesired toxic metabolites. For example, if a toxic metabolite arises through oxidative carbon-hydrogen (C—H) bond cleavage, it can reasonably be assumed that the deuterated analogue will greatly diminish or eliminate production of the unwanted metabolite, even if the particular oxidation is not a rate-determining step. Further information on the state of the art with respect to deuterium-hydrogen exchange may be found, for example in Hanzlik et al., J. Org. Chem. 55, 3992-3997, 1990, Reider et al., J. Org. Chem. 52, 3326-3334, 1987, Foster, Adv. Drug Res. 14, 1-40, 1985, Gillette et al, Biochemistry 33(10) 2927-2937, 1994, and Jarman et al. Carcinogenesis 16(4), 683-688, 1993.

Compounds of the present invention are, subsequent to their preparation, preferably isolated and purified to obtain a composition containing an amount by weight equal to or greater than 95% (“substantially pure”), which is then used or formulated as described herein. In certain embodiments, the compounds of the present invention are more than 99% pure.

Combinations of substituents and variables envisioned by this invention are only those that result in the formation of stable compounds. The term “stable”, as used herein, refers to compounds which possess stability sufficient to allow manufacture and which maintains the integrity of the compound for a sufficient period of time to be useful for the purposes detailed herein (e.g., therapeutic or prophylactic administration to a subject).

The recitation of a listing of chemical groups in any definition of a variable herein includes definitions of that variable as any single group or combination of listed groups. The recitation of an embodiment for a variable herein includes that embodiment as any single embodiment or in combination with any other embodiments or portions thereof.

### DETAILED DESCRIPTION OF THE INVENTION

The invention provides novel, selective and potent MetAP-2 inhibitors, methods of their synthesis, and pharmaceutical compositions as well as methods thereof for treating, preventing or reducing various diseases or conditions associated with MetAP-2, for example, cancer, obesity, diabetes rheumatoid arthritis and psoriasis.

MetAP-2 inhibitors of the invention are orally and/or topically available and are suitable for oral or topical administrations. These compounds are designed to show good potency against MetAP-2 with good oral absorption and good in vivo stability.

In one aspect, the invention generally relates to a compound having the structural formula of (I), or a pharmaceutically acceptable form or an isotope derivative thereof:

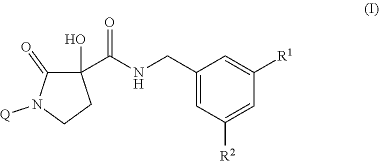

wherein

each of R¹ and R² is independently selected from F, Cl, Br, and I;

Q is (a) an unsubstituted or substituted 5- or 6-membered aromatic ring, or (b) an unsubstituted or substituted 5- or 6-membered aromatic ring fused with a second unsubstituted or substituted 5- or 6-membered ring.

In certain embodiments of (I), the compound has the structural formula of (I^{A}):

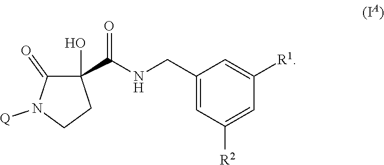

In certain embodiments of (I^{A}), Q is an unsubstituted or substituted 5- or 6-membered aromatic ring Q¹ and the compound has the structural formula (I^{A}₁):

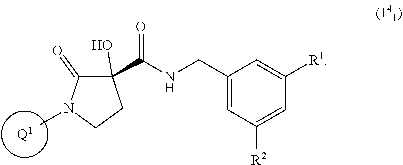

In certain embodiments of (I^{A}₁), Q¹ is a substituted 6-membered aromatic ring.

In certain embodiments, the compound has the following structural formula (II^{A}):

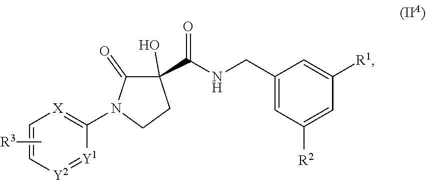

wherein

X is N or CH;

each of Y¹ and Y² is independently N or CH, provided that if one of Y¹ and Y² is N, the other is CH;

R³ is a group comprising a P(═O)(R′)(R″) group or a S(R′)(R″)(R″′) group; and each of R′, R″, and R″′ is independently selected from H and C₁₋₆ alkyl.

In certain embodiments, R³ is at the para-position and the compound has having the structural formula (II^{A}₁):

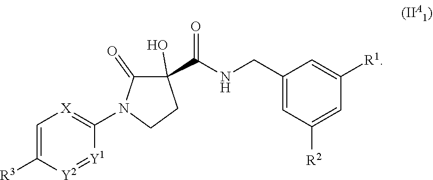

In certain embodiments, R³ is at the meta-position and the compound has the structural formula (II^{A}₂):

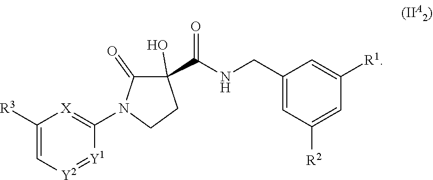

In certain embodiments, each of X, Y¹ and Y² is CH.

In certain embodiments, one of X, Y¹ and Y² is not CH.

In certain embodiments, X is CH, Y¹ is CH, and Y² is N.

In certain embodiments, X is N, Y¹ is CH, and Y² is N.

In certain embodiments, X is CH, Y¹ is N, and Y² is CH.

In certain embodiments, R³ is group comprising a P(═O)(R′)(R′) group. In certain embodiments, R′ and R″ are the same, each being a C₁₋₂ alkyl. In certain embodiments, R′ and R″ are the same, each being a C₃₄ cycloalkyl.

In certain embodiments, R³ is group comprising a S(R′)(R″)(R″′) group. In certain embodiments, R′, R″ and R″′ are the same, each being a C₁₋₃ alkyl.

In certain embodiments, R³ is —A-P(═O)(R′)(R″), wherein A is selected from a single bond, (CH₂)ₘ, (CH₂)ₘ—NR^{a}, NR^{a} —(CH₂)ₘ, (CH₂)ₘ—O, O—(CH₂)ₘ, (CH₂)ₘ, —C(═O)—, (CH₂)ₘ, (CH₂)ₘ—NR^{a}—(CH₂)ₘ, in which m is 0, 1, 2, 3, or 4, and R^{a} is H or a C₁₋₆ alkyl. In certain embodiments, A is a single bond such that P(═O)(R′)(R″) is bonded to Q¹ via the single bond.

In certain embodiments of (I^{A}), Q is a substituted or unsubstituted 5- or 6-membered aromatic ring Q¹ fused with a second 5- or 6-membered ring Q² and the compound has the structural formula (I^{A}₂):

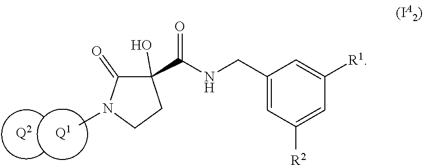

In certain embodiments of (I^{A}₂), Q¹ is a 6-membered aromatic ring and Q² is a 5-membered ring, having the following structural formula (III^{A}):

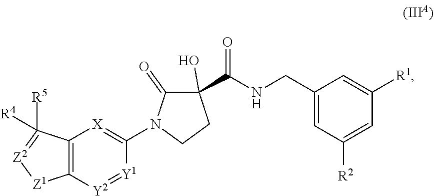

wherein

X is N or CH;

each of Y¹ and Y² is independently N or CH, provided that if one of Y¹ and Y² is N, the other is CH;

Z¹ is NH or CH₂;

Z² is C═O, S(═O)₂, CR′ or CR′R″ wherein R′ and R″ is independently H or a C₁₋₃ alkyl;

--- is absent when Z² is C═O, S(═O)₂ or CR′R″, or represents a bond when Z² is CR′, in which R⁵ is absent; and

R⁴ and R⁵, if present, is independently selected from H and a C₁₋₆ alkyl, and optionally R⁴ and R⁵, together with the carbon atom to which they are attached, form a 3- to 6-membered aliphatic ring.

In certain embodiments of (III^{A}), Z¹ is NH and Z² is CH and the compound has the structural formula (III^{A}₁):

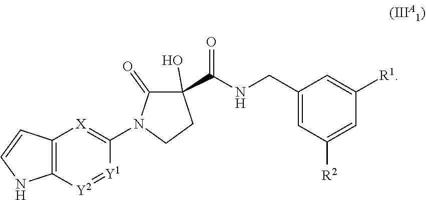

In certain embodiments of (III^{A}) Z¹ is NH and Z² is CH₂ and the compound has the structural formula (III^{A}₂):

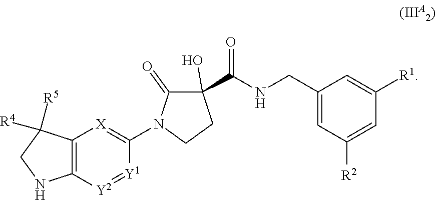

In certain embodiments of (III^{A}) Z¹ is NH and Z² is C═O and the compound has the structural formula (III^{A}₃):

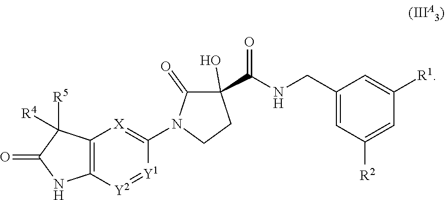

In certain embodiments of (III^{A}), Z¹ is NH and Z² is S(═O)₂ and the compound has the structural formula (III^{A}₄):

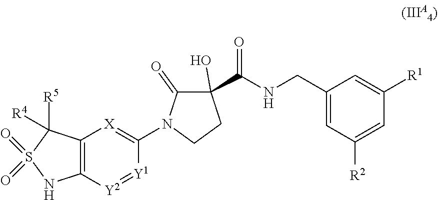

In certain embodiments, each of X, Y¹ and Y² is CH.

In certain embodiments, one of X, Y¹ and Y² is not CH.

In certain embodiments, X is CH, Y¹ is CH, and Y² is N.

In certain embodiments, X is N, Y¹ is CH, and Y² is N.

In certain embodiments, X is CH, Y¹ is N, and Y² is CH.

In certain embodiments of (I^{A}₂), Q¹ is a 5-membered aromatic ring and Q² is a 5-membered ring, having the following structural formula (IV^{A}):

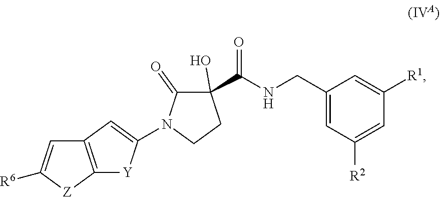

wherein
Y is S or O;
Z is independently N or CH; and
R⁶ is H, alkyl, COOH, or an amide group.

In certain embodiments, Y is S and Z is NH.

In certain embodiments of (I), the compound has the structural formula of (I^{B}):

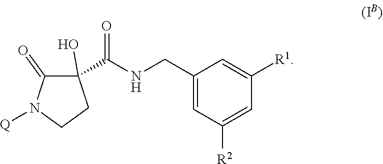

In certain embodiments of (I^{B}), Q is an unsubstituted or substituted 5- or 6-membered aromatic ring Q¹ and the compound has the structural formula (I^{B}₁):

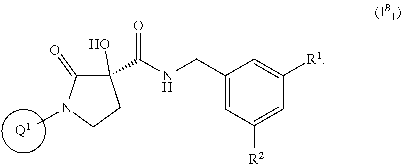

In certain embodiments of (I^{B}₁), Q¹ is a substituted 6-membered aromatic ring.

In certain embodiments of (I^{B}₁), the compound has the following structural formula (II^{A}):

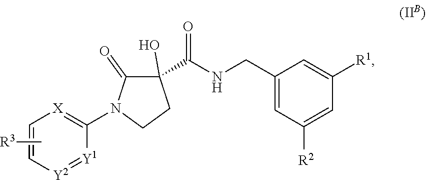

wherein
X is N or CH;
each of Y¹ and Y² is independently N or CH, provided that if one of Y¹ and Y² is N, the other is CH;
R³ is a group comprising a P(═O)(R′)(R″) group or a S(R′)(R″)(R″′) group; and
each of R′, R″, and R″′ is independently selected from H and C₁₋₆ alkyl.

In certain embodiments of (II^{B}), R³ is at the para-position and the compound has the structural formula (II^{B}₁):

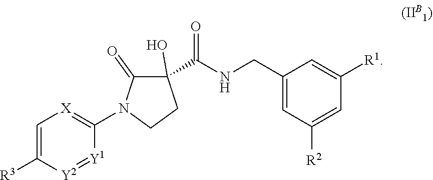

In certain embodiments of (II^{B}) R³ is at the meta-position and the compound has the structural formula (II^{B}₂):

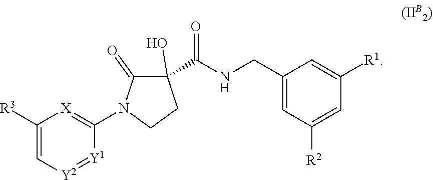

In certain embodiments, each of X, Y¹ and Y² is CH.

In certain embodiments, one of X, Y¹ and Y² is not CH.

In certain embodiments, X is CH, Y¹ is CH, and Y² is N.

In certain embodiments, X is N, Y¹ is CH, and Y² is N.

In certain embodiments, X is CH, Y¹ is N, and Y² is CH.

In certain embodiments, R³ is group comprising a P(═O)(R′)(R″) group. In certain embodiments, R′ and R″ are the same, each being a C₁₋₂ alkyl. In certain embodiments, R′ and R″ are the same, each being a C₃₋₄ cycloalkyl.

In certain embodiments, R³ is group comprising a S(R′)(R″)(R″′) group. In certain embodiments, R′, R″ and R″′ are the same, each being a C₁₋₃ alkyl.

In certain embodiments, R³ is —A-P(═O)(R′)(R″), wherein A is selected from a single bond, (CH₂)ₘ, (CH₂)ₘ—NR^{a}, NR^{a} —(CH₂)ₘ, (CH₂)ₘ—O, O—(CH₂)ₘ, (CH₂)ₘ—C(═O)—, (CH₂)ₘ, (CH₂)ₘ—NR^{a}—(CH₂)ₘ, in which m is 0, 1, 2, 3, or 4, and R^{a} is H or a C₁₋₆ alkyl. In certain embodiments, A is a single bond such that P(═O)(R′)(R″) is bonded to Q¹ via the single bond.

In certain embodiments of (I^{B}), Q is a substituted or unsubstituted 5- or 6-membered aromatic ring Q¹ fused with a second 5- or 6-membered ring Q², having the structural formula

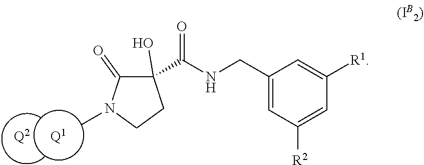

In certain embodiments of (I^{B}₂), Q¹ is a 6-membered aromatic ring and Q² is a 5-membered ring and the compound has the following structural formula (III^{B}

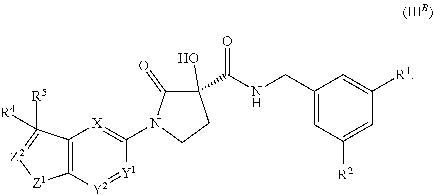

wherein
X is N or CH;
each of Yⁱ and Y² is independently N or CH, provided that if one of Yⁱ and Y² is N, the other is CH;
Z¹ is NH or CH₂;
Z² is C═O, S(═O)₂, CR′ or CR′R″ wherein R′ and R″ is independently H or a C₁₋₃ alkyl;
--- is absent when Z² is C═O, S(═O)₂ or CR′R″, or represents a bond when Z² is CR′, in which R⁵ is absent; and
R⁴ and R⁵, if present, is independently selected from H and a C₁₋₆ alkyl, and optionally R⁴ and R⁵, together with the carbon atom to which they are attached, form a 3- to 6-membered aliphatic ring.

In certain embodiments of (III^{B}), Z¹ is NH and Z² is CH and the compound has the structural formula (III^{B}₁):

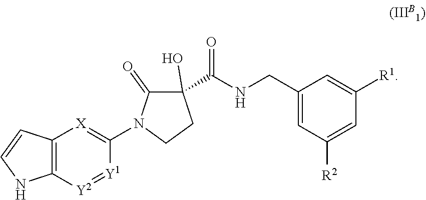

In certain embodiments of (III^{B}), Z^{I} is NH and Z² is CH₂ and the compound has the structural formula (III^{B}₂):

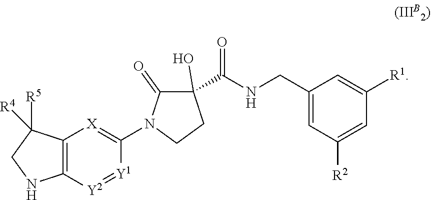

In certain embodiments of (III^{B}), Z^{I} is NH and Z² is C═O and the compound has the structural formula (III^{B}₃):

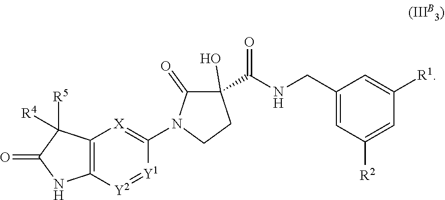

In certain embodiments of (III^{B}), Z¹ is NH and Z² is S(═O)₂ and the compound has the structural formula (III^{B}₄):

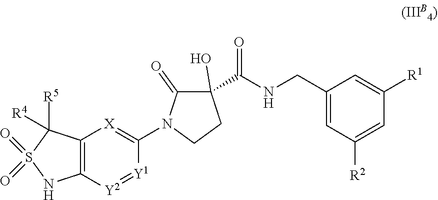

In certain embodiments, each of X, Y¹ and Y² is CH.

In certain embodiments, one of X, Y¹ and Y² is not CH.

In certain embodiments, X is CH, Y¹ is CH, and Y² is N.

In certain embodiments, X is N, Y¹ is CH, and Y² is N.

In certain embodiments, X is CH, Y¹ is N, and Y² is CH.

In certain embodiments of (I^{B}₂), Q¹ is a 5-membered aromatic ring and Q² is a 5-membered ring, having the following structural formula (IV^{B}

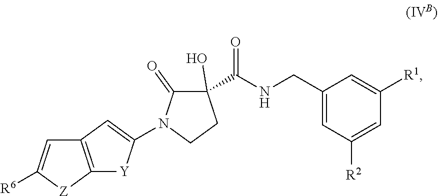

wherein
Y is S or O;
Z is independently N or CH; and
R⁶ is H, alkyl, COOH, or an amide group.

In certain embodiments of (IV^{B}), Y is S and Z is NH.

Non-limiting examples of compounds of the invention include:

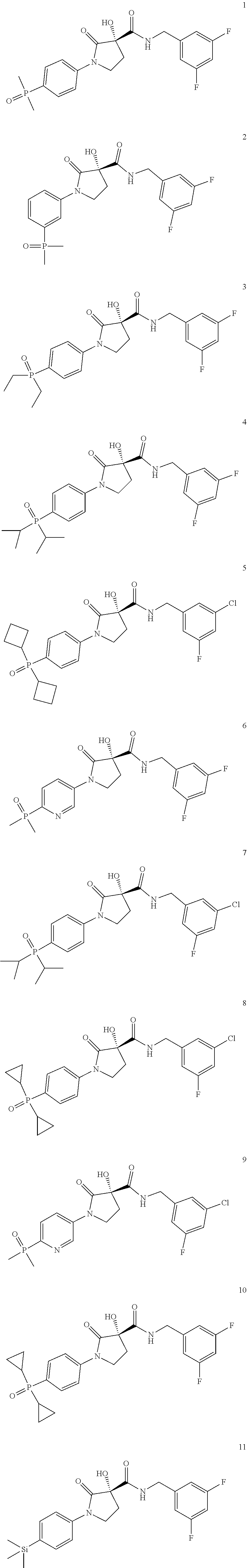

Non-limiting examples of compounds of the invention include:

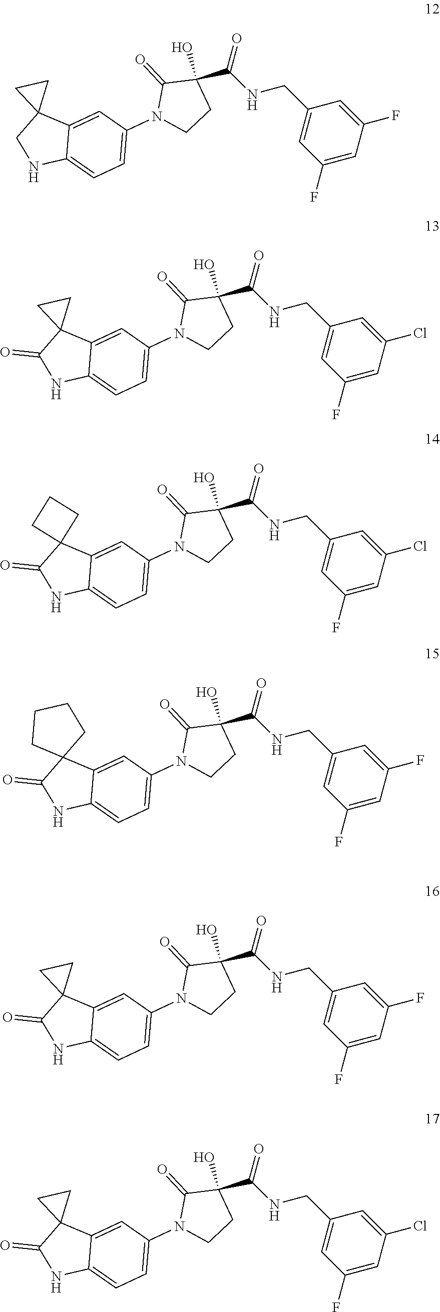 [SMILES: O=C(NCc1cc(F)cc(F)c1)[C@@]1(O)CCN(c2ccc3c(c2)C2(CC2)CN3)C1=O ; O=C1Nc2ccc(N3CC[C@](O)(C(=O)NCc4cc(F)cc(Cl)c4)C3=O)cc2C12CC2 ; O=C1Nc2ccc(N3CC[C@](O)(C(=O)NCc4cc(F)cc(Cl)c4)C3=O)cc2C12CCC2 ; O=C1Nc2ccc(N3CC[C@](O)(C(=O)NCc4cc(F)cc(F)c4)C3=O)cc2C12CCCC2 ; O=C1Nc2ccc(N3CC[C@](O)(C(=O)NCc4cc(F)cc(F)c4)C3=O)cc2C12CC2 ; O=C1Nc2ccc(N3CC[C@](O)(C(=O)NCc4cc(F)cc(Cl)c4)C3=O)cc2C12CC2]

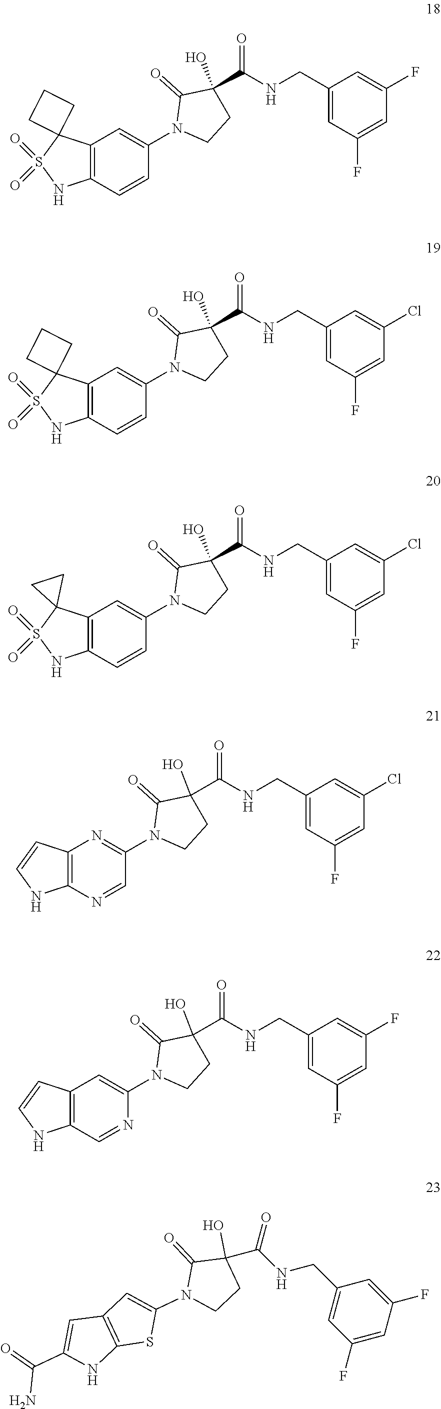 [SMILES: O=C(NCc1cc(F)cc(F)c1)[C@@]1(O)CCN(c2ccc3c(c2)C2(CCC2)S(=O)(=O)N3)C1=O ; O=C(NCc1cc(F)cc(Cl)c1)[C@@]1(O)CCN(c2ccc3c(c2)C2(CCC2)S(=O)(=O)N3)C1=O ; O=C(NCc1cc(F)cc(Cl)c1)[C@@]1(O)CCN(c2ccc3c(c2)C2(CC2)S(=O)(=O)N3)C1=O ; O=C(NCc1cc(F)cc(Cl)c1)C1(O)CCN(c2cnc3[nH]ccc3n2)C1=O ; O=C(NCc1cc(F)cc(F)c1)C1(O)CCN(c2cc3cc[nH]c3cn2)C1=O ; NC(=O)c1cc2cc(N3CCC(O)(C(=O)NCc4cc(F)cc(F)c4)C3=O)sc2[nH]1]

In another aspect, the invention generally relates to a pharmaceutical composition comprising a compound disclosed herein and a pharmaceutically acceptable excipient, carrier, or diluent.

In another aspect, the invention generally relates to a pharmaceutical composition comprising an amount of a compound having the structural formula (I), or a pharmaceutically acceptable form or an isotope derivative thereof:

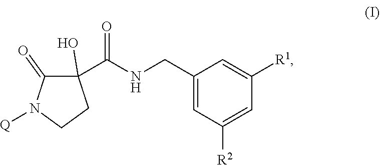

wherein

each of R¹ and R² is independently selected from F, Cl, Br, and I; Q is
(a) an unsubstituted or substituted 5- or 6-membered aromatic ring, or
(b) an unsubstituted or substituted 5- or 6-membered aromatic ring fused with a second unsubstituted or substituted 5- or 6-membered ring.

In certain embodiments of the pharmaceutical composition, the compound of (I) has the structural formula (I^{A}):

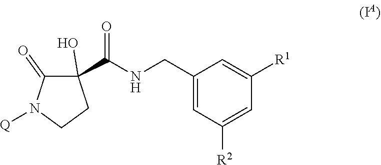

In certain embodiments of the pharmaceutical composition, the compound of (I) has the structural formula (I^{B}):

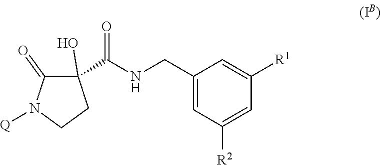

In certain embodiments, the pharmaceutical composition of the invention is effective to treat, prevent, or reduce a disease or condition selected from cancer, obesity, diabetes, rheumatoid arthritis, psoriasis, or a related disease or condition. In certain embodiments, the pharmaceutical composition of the invention is effective to treat, prevent, or reduce cancer, or a related disease or condition. In certain embodiments, the pharmaceutical composition of the invention is effective to treat, prevent, or reduce obesity, or a related disease or condition. In certain embodiments, the pharmaceutical composition of the invention is effective to treat, prevent, or reduce diabetes, or a related disease or condition. In certain embodiments, the pharmaceutical composition of the invention is effective to treat, prevent, or reduce rheumatoid arthritis, or a related disease or condition. In certain embodiments, the pharmaceutical composition of the invention is effective to treat, prevent, or reduce psoriasis, or a related disease or condition.

In yet another aspect, the invention generally relates to a unit dosage form comprising a pharmaceutical composition of the invention.

In yet another aspect, the invention generally relates to a method for treating, reducing, or preventing a disease or condition, comprising administering to a subject in need thereof a pharmaceutical composition comprising a compound having the structural formula of (I), or a pharmaceutically acceptable form or an isotope derivative thereof:

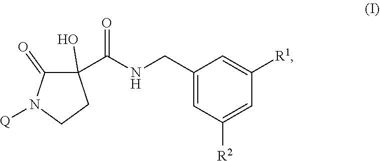

wherein

each of R¹ and R² is independently selected from F, Cl, Br, and I; Q is
(a) an unsubstituted or substituted 5- or 6-membered aromatic ring, or
(b) an unsubstituted or substituted 5- or 6-membered aromatic ring fused with a second unsubstituted or substituted 5- or 6-membered ring,
   effective to treat, prevent, or reduce one or more of cancer, obesity, diabetes rheumatoid arthritis, psoriasis, or a related disease or condition thereof, in a mammal, including a human, and a pharmaceutically acceptable excipient, carrier, or diluent.

In certain embodiments of the method, the compound has the structural formula (I^{A}):

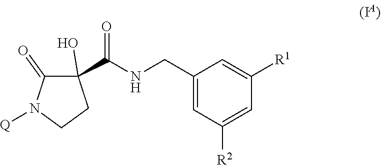

In certain embodiments of the method, the compound has the structural formula (I^{B}):

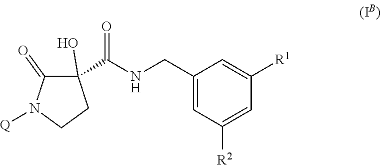

In yet another aspect, the invention generally relates to a method for treating, preventing or reducing a disease or condition treatable by modulation, regulation, or inhibition of methionine aminopeptidase (MetAP-2), comprising administering to a subject in need thereof an effective amount of a compound disclosed herein.

In certain embodiments, the method of the invention is effective to treat, prevent, or reduce a disease or condition selected from cancer, obesity, diabetes rheumatoid arthritis, psoriasis, or a related disease or condition. In certain embodiments, the method of the invention is effective to treat, prevent, or reduce cancer, or a related disease or condition. In certain embodiments, the method of the invention is effective to treat, prevent, or reduce obesity, or a related disease or condition. In certain embodiments, the method of the invention is effective to treat, prevent, or reduce diabetes, or a related disease or condition. In certain embodiments, the method of the invention is effective to treat, prevent, or reduce rheumatoid arthritis, or a related disease or condition. In certain embodiments, the method of the invention is effective to treat, prevent, or reduce psoriasis, or a related disease or condition.

In certain embodiments, the method of the invention further comprises administering the subject a second therapeutic agent.

In yet another aspect, the invention generally relates to use of a compound of the invention for treating or reducing a disease or condition.

In yet another aspect, the invention generally relates to use of a compound of the invention, and a pharmaceutically acceptable excipient, carrier, or diluent, in preparation of a medicament for treating or reducing a disease or condition.

In certain embodiments of use of a compound of the invention, the disease or condition being treated or reduced is cancer, obesity, diabetes rheumatoid arthritis, psoriasis, or a related disease or condition thereof

In certain embodiments of use of a compound of the invention, the disease or condition being treated or reduced is cancer, or a related disease or condition.

In certain embodiments of use of a compound of the invention, the disease or condition being treated or reduced is obesity, or a related disease or condition.

In certain embodiments of use of a compound of the invention, the disease or condition being treated or reduced is diabetes, or a related disease or condition.

In certain embodiments of use of a compound of the invention, the disease or condition being treated or reduced is rheumatoid arthritis, or a related disease or condition.

In certain embodiments of use of a compound of the invention, the disease or condition being treated or reduced is psoriasis, or a related disease or condition.

In certain embodiments, compounds according to the invention may be used in the treatment or reduction of any carcinoma having a substantial degree of vascularization, such as lung, breast, prostate, head and neck, oesophageal, pancreatic, liver, colon or kidney carcinomas or carcinomas that induce metastases, such as colon, breast, liver, head and neck, and stomach carcinomas, and melanomas. These compounds may be used in monotherapy or combination with radiotherapy or chemotherapy.

In certain embodiments, compounds according to the inventions may also be used in the treatment or reduction of hepatocarcinomas, cholangiocarcinoma and malignant mesothelioma, pancreatic cancer, head and neck cancer, and haemoangioma.

In certain embodiments, compounds according to the inventions may also be used in the treatment or reduction of type II diabetes, nonalcoholic steatohepatitis, obesity, or to provide therapeutic weight loss.

Compositions of the present invention are administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. Preferably, the compositions are administered orally, intraperitoneally or intravenously. Sterile injectable forms of the compositions of this invention include aqueous or oleaginous suspension. These suspensions are formulated according to techniques known in the art using suitable dispersing or wetting agents and suspending agents. The sterile injectable preparation is also be a sterile injectable solution or suspension in a non-toxic parenterally acceptable diluent or solvent, for example as a solution in 1,3-butanediol. Among the acceptable vehicles and solvents that are employed are water, Ringer's solution and isotonic sodium chloride solution. In addition, sterile, fixed oils are conventionally employed as a solvent or suspending medium.

For this purpose, any bland fixed oil employed includes synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms are also be used for the purposes of formulation.

Pharmaceutically acceptable compositions of this invention are orally administered in any orally acceptable dosage form. Exemplary oral dosage forms are capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents are optionally also added.

Alternatively, pharmaceutically acceptable compositions of this invention are administered in the form of suppositories for rectal administration. These can be prepared by mixing the agent with a suitable non-irritating excipient that is solid at room temperature but liquid at rectal temperature and therefore will melt in the rectum to release the drug. Such materials include cocoa butter, beeswax and polyethylene glycols.

Pharmaceutically acceptable compositions of this invention are also administered topically, especially when the target of treatment includes areas or organs readily accessible by topical application, including diseases of the eye, the skin, or the lower intestinal tract. Suitable topical formulations are readily prepared for each of these areas or organs.

Topical application for the lower intestinal tract can be effected in a rectal suppository formulation (see above) or in a suitable enema formulation. Topically-transdermal patches are also used.

For topical applications, provided pharmaceutically acceptable compositions are formulated in a suitable ointment containing the active component suspended or dissolved in one or more carriers. Exemplary carriers for topical administration of compounds of this aremineral oil, liquid petrolatum, white petrolatum, propylene glycol, polyoxyethylene, polyoxypropylene compound, emulsifying wax and water. Alternatively, provided pharmaceutically acceptable compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

Pharmaceutically acceptable compositions of this invention are optionally administered by nasal aerosol or inhalation. Such compositions are prepared according to techniques well-known in the art of pharmaceutical formulation and are prepared as solutions in saline, employing benzyl alcohol or other suitable preservatives, absorption promoters to enhance bioavailability, fluorocarbons, and/or other conventional solubilizing or dispersing agents.

Most preferably, pharmaceutically acceptable compositions of this invention are formulated for oral administration. Such formulations may be administered with or without food. In some embodiments, pharmaceutically acceptable compositions of this invention are administered without food. In other embodiments, pharmaceutically acceptable compositions of this invention are administered with food.

The amount of compounds of the present invention that are optionally combined with the carrier materials to produce a composition in a single dosage form will vary depending upon the host treated, the particular mode of administration. Preferably, provided compositions should be formulated so that a dosage of between 0.01-100 mg/kg body weight/day of the compound can be administered to a patient receiving these compositions.

It should also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated. The amount of a compound of the present invention in the composition will also depend upon the particular compound in the composition.

### Examples

As depicted in the Examples below, in certain exemplary embodiments, compounds are prepared according to the following general procedures. It will be appreciated that, although the general methods depict the synthesis of certain compounds of the present invention, the following general methods, and other methods known to one of ordinary skill in the art, can be applied to all compounds and subclasses and species of each of these compounds, as described herein.

Compound numbers utilized in the Examples below correspond to compound numbers set forth supra.

¹H was recorded at 400 MHz on a Varian Mercury 400 spectrometer. ¹³C NMR was recorded at 100 MHz. Proton chemical shifts were internally referenced to the residual proton resonance in CDCl3 (7.26 ppm). Carbon chemical shifts were internally referenced to the deuterated solvent signals in CDCl3 (77.20 ppm).

LC-MS spectra were recorded on a Shimadzu LC-MS2020 using Agilent C18 column (Eclipse XDB-C18, 5 um, 2.1×50 mm) with flow rate of 1 mL/min. Mobile phase A: 0.1% of formic acid in water; mobile phase B: 0.1% of formic acid in acetonitrile. A general gradient method was used.

TABLE 1

| Time (min) | A | B |
|---|---|---|
| 0 | 95 | 5 |
| 3 | 0 | 100 |
| 4 | 0 | 100 |
| 4.05 | 95 | 5 |

Analytical HPLC was performed on Agilent 1200 HPLC with a Zorbax Eclipse XDB C18 column (2.1×150 mm) with flow rate of 1 mL/min. Mobile phase A: 0.1% of TFA in water; mobile phase B: 0.1% of TFA in acetonitrile. A general method with following gradient was used.

TABLE 2

| Time (min) | Mobile Phase A | Mobile Phase B |
|---|---|---|
| 0 | 95 | 5 |
| 15 | 0 | 100 |
| 16 | 0 | 100 |
| 16.5 | 95 | 5 |
| 16.5 | | stop |

Preparative HPLC was performed on Varian ProStar using Hamilton C18 PRP-1 column (15×250 mm) with flow rate of 20 mL/min. Mobile phase A: 0.1% of TFA in water; mobile phase B: 0.1% of TFA in acetonitrile. A typical gradient method was used.

TABLE 3

| Time (min) | Mobile Phase A | Mobile Phase B |
|---|---|---|
| 0 | 90 | 10 |
| 30 | 30 | 70 |
| 35 | 10 | 100 |
| 40 | 90 | 10 |
| 45 | | stop |

### Example 1

### (S)-N-(3,5-difluorobenzyl)-1-(4-(dimethylphosphoryl)phenyl)-3-hydroxy-2-oxopyrrolidine-3-arboxamide (1)

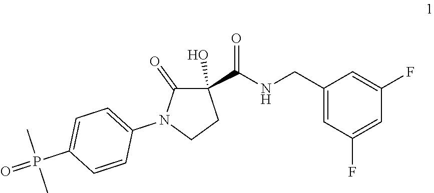 [SMILES: CP(C)(=O)c1ccc(N2CC[C@](O)(C(=O)NCc3cc(F)cc(F)c3)C2=O)cc1]

### Scheme 1

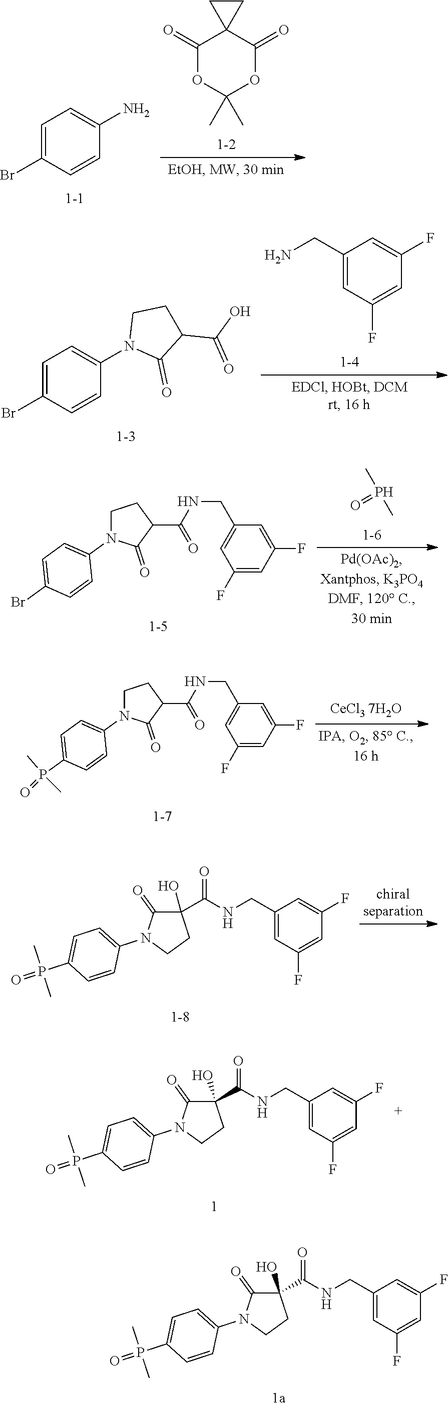

Step 1: To a mixture of 4-bromoaniline (1-1) (500 mg, 2.9 mmol, 1.0 eq) in EtOH (5 mL) was added 6,6-dimethyl-5,7-dioxaspiro[2.5]octane-4,8-dione (1-2) (741 mg, 4.36 mmol, 1.5 eq). The mixture was refluxed at 100° C. in MW for 30 min. TLC analysis of the reaction mixture showed full conversion to the desired product. Then the mixture was filtered and the residue was purified by column chromatography on a silica gel (DCM:MeOH, 1:1) to afford 1-(4-bromophenyl)-2-oxopyrrolidine-3-carboxylic acid (1-3) (670 mg, 82%). TLC (DCM:MeOH, 10:1): R_{f}=0.2.

Step 2: To a mixture of 1-(4-bromophenyl)-2-oxopyrrolidine-3-carboxylic acid (1-3) (670 mg, 2.37 mmol, 1.0 eq) in DCM (5 mL) was added (3,5-difluorophenyl)methanamine (1-4) (338 mg, 2.37 mmol, 1.0 eq), EDCI (590 mg, 3.08 mmol, 1.3 eq), HOBt (989 mg, 3.08 mmol, 1.3 eq) and DIEA (917 mg, 7.11 mmol, 3.0 eq). The mixture was stirred at rt overnight. TLC analysis of the reaction mixture showed full conversion to the desired product. The residue was dried over Na₂SO₄ and concentrated under reduced pressure. The residue was purified by column chromatography on a silica gel (PE:EA, 3:1-1:1) to afford 1-(4-bromophenyl)-N-(3,5-difluorobenzyl)-2-oxopyrrolidine-3-carboxamide (1-5) (590 mg, 61%) as white solid. TLC (PE:EA, 1:1): R_{f}=0.6.

Step 3: To a mixture of 1-(4-bromophenyl)-N-(3,5-difluorobenzyl)-2-oxopyrrolidine-3-carboxamide (1-5) (300 mg, 0.735 mmol, 1.0 eq) in DMF (3 mL) was added dimethylphosphine oxide (1-6) (63 mg, 0.808 mmol, 1.1 eq), Pd(OAc)₂ (8.25 mg, 0.036 mmol, 0.05 eq), Xantphos (25.5 mg, 0.0225 mmol, 0.06 eq) and K₃PO₄ (215 mg, 0.808 mmol, 1.1 eq). The mixture was stirred at 120° C. for 30 min in MW. TLC analysis of the reaction mixture showed full conversion to the desired product. The residue was dried over Na₂SO₄ and concentrated under reduced pressure. The residue was purified by column chromatography on a silica gel (DCM ˜ DCM: MeOH, 30:1) to afford N-(3,5-difluorobenzyl)-1-(4-(dimethylphosphoryl)phenyl)-2-oxopyrrolidine-3-carboxamide (1-7) (200 mg, 67%). TLC (DCM:MeOH, 10:1): R_{f}=0.8.

Step 4: To a mixture of N-(3,5-difluorobenzyl)-1-(4-(dimethylphosphoryl)phenyl)-2-oxopyrrolidine-3-carboxamide (1-7) (200 mg, 0.49 mmol, 1.0 eq) in IPA (3 mL) was added CeCl₃ 7H₂O (73.4 mg, 0.196 mmol, 0.4 eq). The mixture was stirred at 85° C. overnight under O₂. TLC analysis of the reaction mixture showed full conversion to the desired product. The residue was dried over Na₂SO₄ and concentrated under reduced pressure. The residue was purified by column chromatography on a silica gel (DCM: MeOH, 20:1) and prep-HPLC to afford N-(3, 5-difluorobenzyl)-1-(4-(dimethylphosphoryl)phenyl)-3-hydroxy-2-oxopyrrolidine-3-carbox amide (1-8) (25 mg, 12%) as a yellow solid. TLC (DCM:MeOH, 10:1): R_{f}=0.3. ¹H NMR (DMSO, 400 MHz): δ 8.72-8.69 (t, J=6.2 Hz, 1H), 7.81-7.73 (m, 4H), 7.06-7.01 (m, 1H), 6.96-6.94 (d, J=6.4 Hz, 2H), 4.39-4.34 (dd, J=16.0 Hz, 6.6 Hz, 1H), 4.25-4.20 (dd, J=15.6 Hz, 5.8 Hz, 1H), 3.89-3.86 (t, J=6.6 Hz, 2H), 2.59 (m, 1H), 2.14-2.11 (m, 1H), 1.62 (s, 3H), 1.59 (s, 3H). m/z: 423.2 [M+H]⁺

Step 5: N-(3, 5-difluorobenzyl)-1-(4-(dimethylphosphoryl)phenyl)-3-hydroxy-2-oxopyrrolidine-3-carbox amide (1-8) (50 mg) was applied to a Chiralcel OD column (5×25 cm 20 m) at a flow of 100 mL /min with solvent system of n-heptane/ethanol=70:30. (S)-Enantiomer (compound 1) was collected in the first fraction, which was concentrated to give (S)-N-(3, 5-difluorobenzyl)-1-(4-(dimethylphosphoryl)phenyl)-3-hydroxy-2-oxopyrrolidine-3-car boxamide (1). Enantiomer 1a was collected in the second fraction, which was concentrated to give

(R)-N-(3, 5-difluorobenzyl)-1-(4-(dimethylphosphoryl)phenyl)-3-hydroxy-2-oxopyrrolidine-3-car boxamide (1a).

### Example 2

### (S)-N-(3,5-difluorobenzyl)-1-(3-(dimethylphosphoryl)phenyl)-3-hydroxy-2-oxopyrrolidine-3 -carboxamide (2)

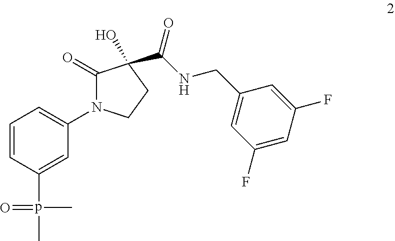

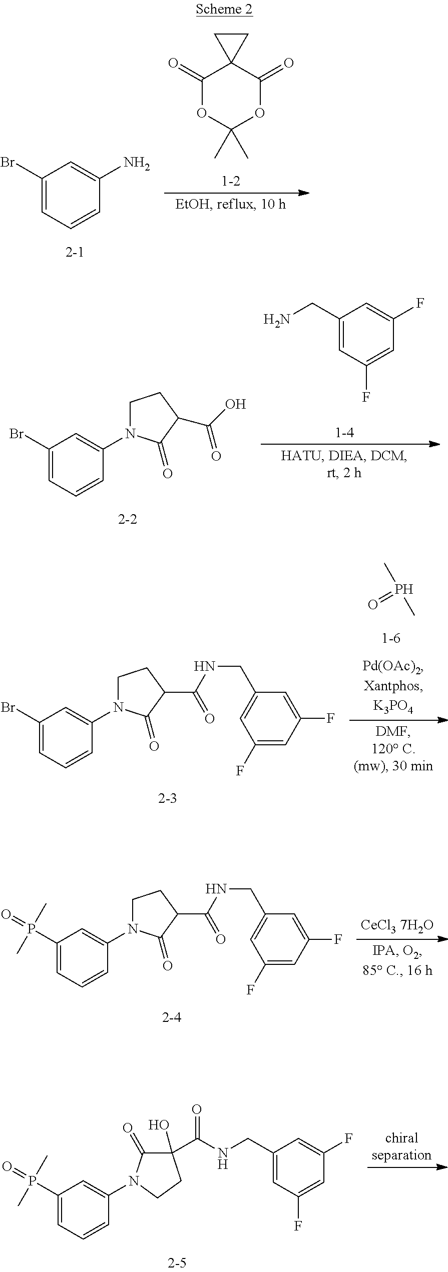

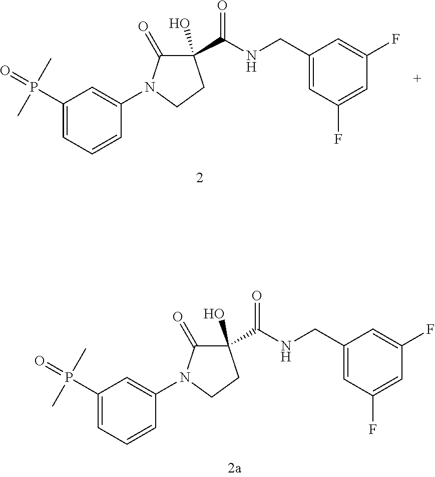 [SMILES: CP(C)(=O)c1cccc(N2CC[C@](O)(C(=O)NCc3cc(F)cc(F)c3)C2=O)c1 ; CP(C)(=O)c1cccc(N2CC[C@@](O)(C(=O)NCc3cc(F)cc(F)c3)C2=O)c1]

Step 1: To a mixture of compound 2-1 (1.0 g, 5.8 mmol) in EtOH (10 mL) was added compound 1-2 (1.29 g, 7.6 mmol). The mixture was refluxed at 100° C. for 10 hrs. TLC analysis of the reaction mixture showed full conversion to the desired product. Then the mixture was concentrated in vacuo and the residue was purified by column chromatography on a silica gel eluting by 25% to 50% EtOAc in petroleum ether to afford compound 2-2 (1.05 g, 3.7 mmol, 64%). TLC (PE:EA=2:1): Rf (compound 2-2)=0.2, Rf (compound 2-1)=0.9.

Step 2: A solution of compound 2-2 (930 mg, 3.27 mmol), compound 1-4 (562 mg, 3.93 mmol), DIPEA (1.26 g, 9.81 mmol) and HATU (1.86 g, 4.91 mmol) in DCM (20 mL) was stirred at rt for 2 hrs. TLC analysis of the reaction mixture showed full conversion to the desired product. The residue was dried over Na₂SO₄ and concentrated under reduced pressure. The residue was purified by column chromatography on a silica gel (PE:EA, 3:1-1:1) to afford compound 2-3 (1.16 g, 2.84 mmol, 87%) as a white solid. TLC (PE:EA, 2:1): Rf (compound 2-2)=0.1, Rf (compound 2-3)=0.3.

Step 3: To a mixture of compound 2-3 (400 mg, 0.98 mmol) in DMF (3 mL) was added compound 1-6 (84 mg, 1.08 mmol), Pd(OAc)₂ (11 mg, 0.05 mmol), Xantphos (34 mg, 0.06 mmol) and K₃PO₄ (288 mg, 1.08 mmol). The mixture was stirred at 120° C. for 30 min in microwave. TLC analysis of the reaction mixture showed full conversion to the desired product. The residue was dried over Na₂SO₄ and concentrated under reduced pressure. The residue was purified by column chromatography on a silica gel eluting by 5% CH₃OH in CH₂Cl2 to afford compound 2-4 (270 mg, 0.67 mmol, 66%). TLC (DCM:MeOH, 10:1): Rf (compound 2-4)=0.4, Rf (compound 2-3)=0.8.

Step 4: To a mixture of compound 2-4 (270 mg, 0.66 mmol) in IPA (15 mL) was added CeCl₃ 7H₂O (99 mg, 0.27 mmol). The mixture was stirred at 85° C. overnight under O₂. TLC analysis of the reaction mixture showed full conversion to the desired product. The residue was dried over Na₂SO₄ and concentrated under reduced pressure. The residue was purified by prep-HPLC to afford 2-5 (99 mg, 0.23 mmol, 36%) as a yellow solid. TLC (DCM:MeOH, 10:1): Rf (compound 2-4)=0.5, Rf (compound 2-5)=0.3. LCMS: [M+1]: 423. ¹H NMR (400 MHz, DMSO) δ 8.75 (t, J=6.4 Hz, 1H), 8.02 (dd, J=13.6, 1.1 Hz, 1H), 7.94-7.86 (m, 1H), 7.62-7.53 (m, 2H), 7.07 (tt, J=9.4, 2.3 Hz, 1H), 7.03-6.96 (m, 2H), 4.41 (dd, J=15.8, 6.7 Hz, 1H), 4.27 (dd, J=15.8, 6.0 Hz, 2H), 3.96-3.88 (m, 2H), 2.63 (dt, J=11.8, 5.7 Hz, 1H), 2.17 (dt, J=13.0, 7.6 Hz, 1H), 1.69 (s, 3H), 1.65 (s, 3H).

Step 5: Similar to the chiral separation procedure for compound 1 and 1a, compound 2-5 was applied to a Chiralcel OD column to afford compound 2 and 2a.

### Example 3

### (S)-1-(4-(diethylphosphoryl)phenyl)-N-(3,5-difluorobenzyl)-3-hydroxy-2-oxopyrrolidine-3-c arboxamide (3)

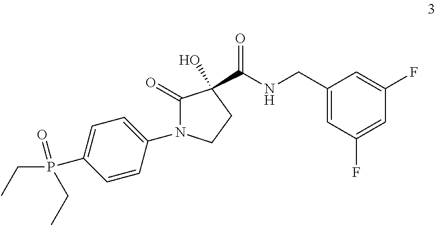 [SMILES: CCP(=O)(CC)c1ccc(N2CC[C@](O)(C(=O)NCc3cc(F)cc(F)c3)C2=O)cc1]

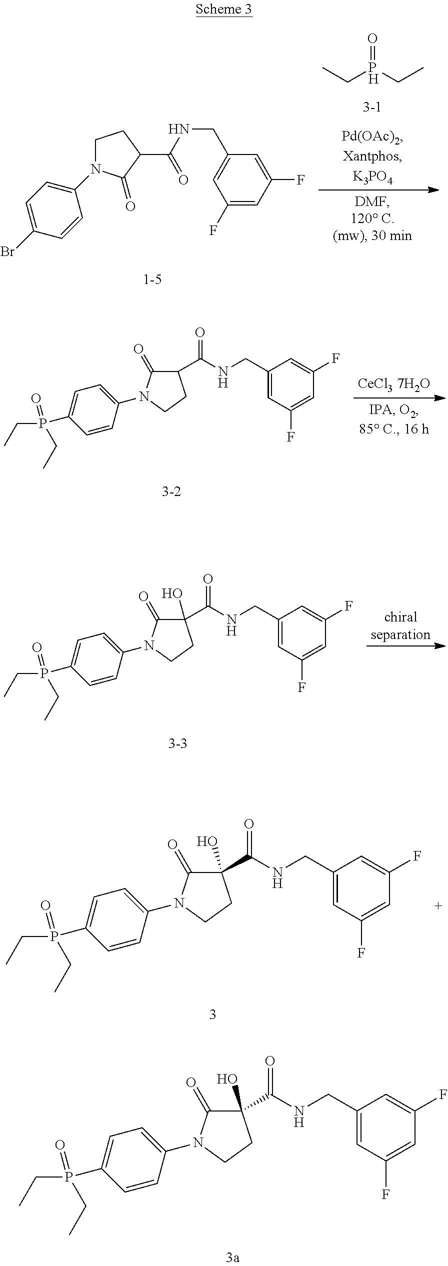

Compound 3 and 3a were prepared using the similar procedure as in compound 2. For compound 3-3. ¹H NMR (400 MHz, CDCl₃) δ 7.74 (d, J=7.2 Hz, 2H), 7.64 (t, J=9.2 Hz, 2H), 7.40 (s, 1H), 6.79 (d, J=5.8 Hz, 2H), 6.69 (s, 1H), 4.44-4.39 (m, 2H), 4.15-4.10 (m, 1H), 3.87-3.81 (m, 1H), 2.81-2.74 (m, 1H), 2.31-2.24 (m, 1H), 2.03-1.84 (m, 4H), 1.18-1.03 (m, 6H). LCMS: [M+1]: 451.

### Example 4

### (S)-N-(3,5-difluorobenzyl)-1-(4-(diisopropylphosphoryl)phenyl)-3-hydroxy-2-oxopyrrolidin e-3-carboxamide (4)

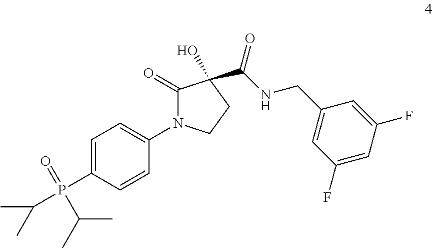 [SMILES: CC(C)P(=O)(c1ccc(N2CC[C@](O)(C(=O)NCc3cc(F)cc(F)c3)C2=O)cc1)C(C)C]

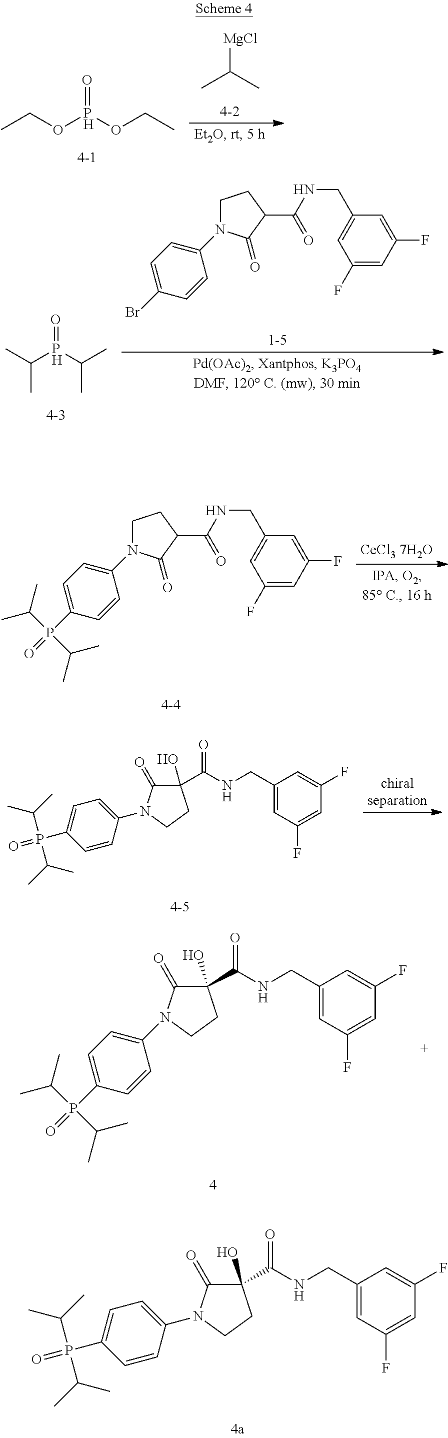

Step 1: Compound 4-2 (20 mL, 2M in THF solution, 40 mmol) was added dropwise to a solution of compound 4-1 (1.38 g, 10 mmol) in anhydrous Ether (30 mL) at 0° C. under N₂. The mixture was stirred at r.t. for 5 hrs. The mixture was quenched by 1M HCl solution (40 mL) at 0° C. Then the mixture was concentrated and the residue was washed with a solution of 10% CH₃OH in CH₂C2. The mixture was filtered and the filtrate was concentrated in vacuo to afford compound 4-3 (600 mg, 4.47 mmol, 44.7%) as colorless oil. TLC (DCM:MeOH, 15:1): Rf (compound 4-1) =0.8, Rf (compound 4-3)=0.5.

Compound 4 and 4a were prepared using the similar procedure as in compound 2. For racemic compound 4-5. ¹H NMR (400 MHz, DMSO-d₆) δ 8.72 (t, J=6.3 Hz, 1H), 7.86-7.79 (m, 2H), 7.66 (t, J=8.8 Hz, 2H), 7.03 (t, J=9.4 Hz, 1H), 6.95 (d, J=6.7 Hz, 2H), 4.28 (ddd, J=21.6, 15.8, 6.3 Hz, 2H), 3.87 (t, J=6.7 Hz, 2H), 2.62-2.51 (m, 1H), 2.30 (dq, J=14.4, 7.2 Hz, 2H), 2.16-1.97 (m, 1H), 2.17-2.04 (m, 1H), 1.01 (dd, J=14.8, 7.0 Hz, 6H), 0.86 (dd, J=15.6, 7.0 Hz, 6H). LCMS: [M+1]: 479

### Example 5

### (S)-N-(3-chloro-5-fluorobenzyl)-1-(4-(dicyclobutylphosphoryl)phenyl)-3-hydroxy-2-oxopyrr olidine-3-carboxamide (5)

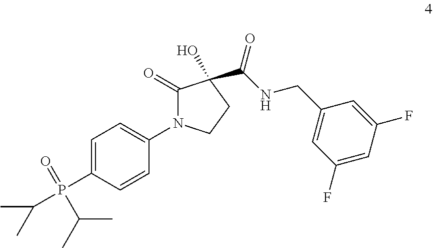 [SMILES: CC(C)P(=O)(c1ccc(N2CC[C@](O)(C(=O)NCc3cc(F)cc(F)c3)C2=O)cc1)C(C)C]

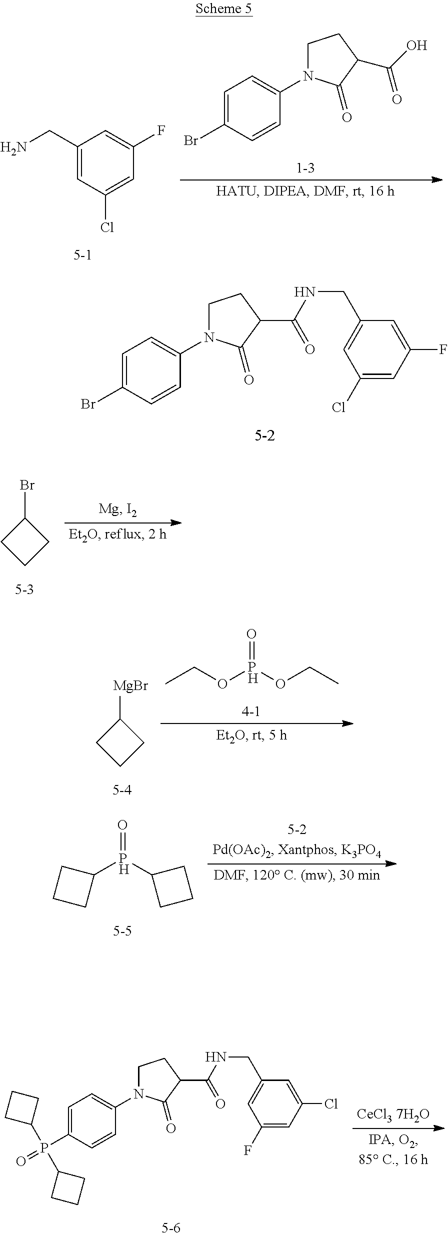

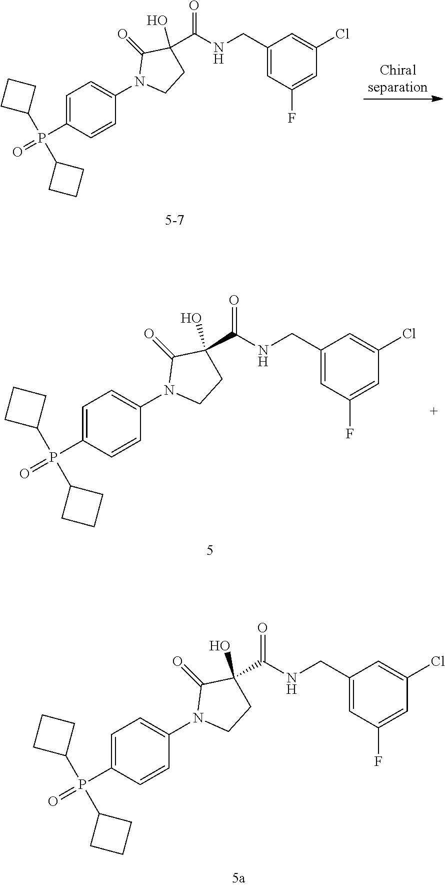 [SMILES: O=C(NCc1cc(F)cc(Cl)c1)C1(O)CCN(c2ccc(P(=O)(C3CCC3)C3CCC3)cc2)C1=O ; O=C(NCc1cc(F)cc(Cl)c1)[C@@]1(O)CCN(c2ccc(P(=O)(C3CCC3)C3CCC3)cc2)C1=O ; O=C(NCc1cc(F)cc(Cl)c1)[C@]1(O)CCN(c2ccc(P(=O)(C3CCC3)C3CCC3)cc2)C1=O]

Step 1: A solution of compound 1-3 (2.5 g, 8.83 mmol), compound 5-1 (1.55 g, 9.71 mmol), DIPEA (3.40 g, 26.5 mmol) and HATU (5.02 g, 13.25 mmol) in DCM (100 mL) was stirred at rt for 2 hrs. TLC analysis of the reaction mixture showed full conversion to the desired product. The residue was dried over Na₂SO₄ and concentrated under reduced pressure. The residue was purified by column chromatography on a silica gel (PE:EA, 3:1-1:1) to afford compound 5-2 (3.0 g, 7.08 mmol, 80%) as a white solid. TLC (PE:EA, 2:1): Rf (compound 1-3)=0.1, Rf (compound 5-2)=0.3.

Step 2 & 3: A 3-neck 1L RBF was fitted with mechanical stirrer, reflux condenser and addition funnel, evacuated/N₂ filled (3 X), charged with Mg powder (2.23 g, 16.49 mmol, 3.3 eq) and I₂ (0.1 mmol), then again evacuated/N₂ filled (3 X). 20 mL of dry Et₂O was then added via canula. To this was added a solution of compound 5-3 (2.23 g, 16.49 mmol, 3.3 eq) in Et₂O (5 mL). After refluxed for 2 hrs, the brown color of the reaction mixture was turned to clear to afford the compound 5-4, which was used directly in the next step. The solution of 5-4 was cooled to 0° C., diethylphosphite 4-1 (690 mg, 5 mmol, 1 eq) in Et₂O (10 mL) was added dropwise over 15 minutes. The mixture was stirred at 25° C. for 18 hrs, then cooled to 0° C. and cautiously quenched with 0.1N HCl solution (10 mL). 50 mL of MTBE was added, and the mixture was agitated well for 5 minutes. The upper organic phase was decanted from the gel and saved. To the remaining gel was added 25 mL CH₂Cl₂, and the mixture agitated well for 5 minutes. The resulted mixture was then filtered through a Celite pad, washing the pad with CH₂Cl₂. the organic phase was combined dried (MgSO4), and concentrated in vacuo. The residue was purified on silica gel eluting by 3% CH₃OH in CH₂Cl₂ to afford compound 5-5 (210 mg, 1.62 mmol, 32% 2steps) as colorless oil. TLC (DCM:MeOH, 10:1): Rf (compound 5-3)=0.5, Rf (compound 5-5)=0.3. ¹H NMR (400 MHz, CDCl₃) δ 7.22-6.03 (m, 1H), 2.69 (d, J=7.9 Hz, 4H), 2.47-1.93 (m, 10H).

Step 3-5: Compound 5 and 5a were prepared using the similar procedure as in compound 2. For compound 5-7, LCMS: [M+1]: 519; ¹H NMR (400 MHz, CDCl₃) δ 7.86 (s, 1H), 7.58 (s, 2H), 7.47 (s, 2H), 7.07 (s, 1H), 6.93 (t, J=9.2 Hz, 2H), 4.47 (dd, J=15.5, 6.5 Hz, 1H), 4.33 (dd, J=15.2, 5.6 Hz, 1H), 4.00 (d, J=7.6 Hz, 1H), 3.77 (s, 1H), 2.92-2.76 (m, 3H), 2.48-2.36 (m, 3H), 2.27-2.05 (d, J=45.8 Hz, 6H), 2.00-1.83 (m, 4H).

### Example 6

### (S)-N-(3,5-difluorobenzyl)-1-(6-(dimethylphosphoryl)pyridin-3-yl)-3-hydroxy-2-oxopyrroli dine-3-carboxamide (6)

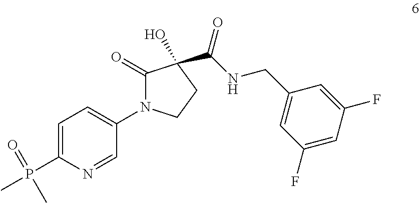 [SMILES: CP(C)(=O)c1ccc(N2CC[C@](O)(C(=O)NCc3cc(F)cc(F)c3)C2=O)cn1]

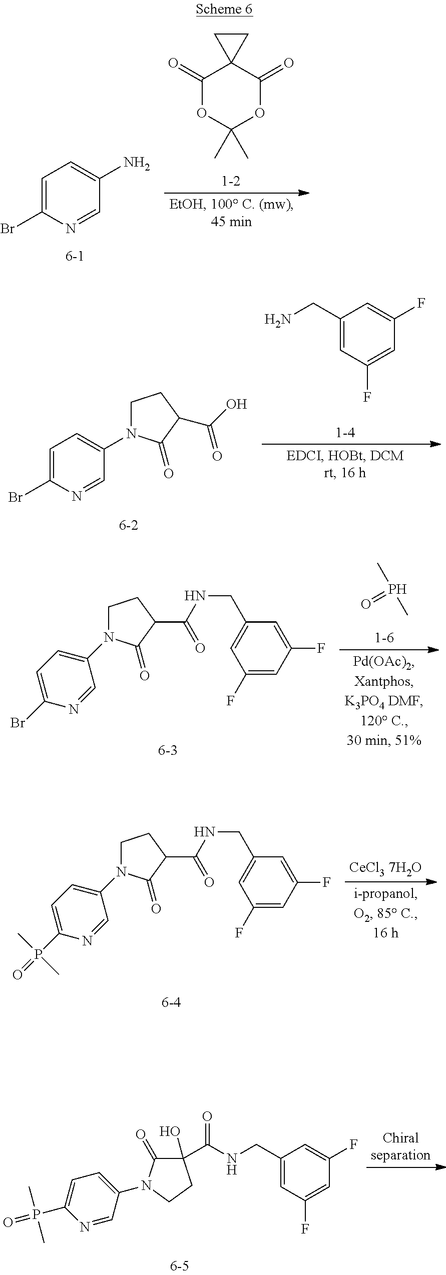

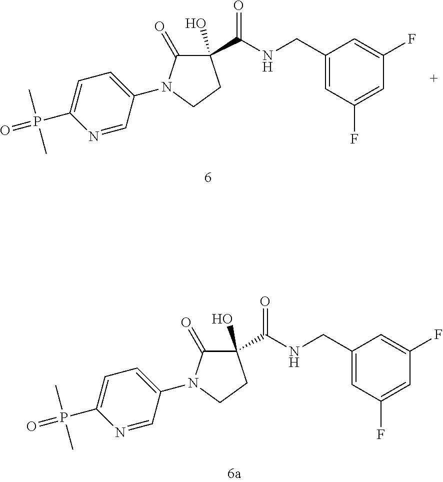 [SMILES: CP(C)(=O)c1ccc(N2CC[C@](O)(C(=O)NCc3cc(F)cc(F)c3)C2=O)cn1 ; CP(C)(=O)c1ccc(N2CC[C@@](O)(C(=O)NCc3cc(F)cc(F)c3)C2=O)cn1]

Step 1: To a mixture of compound 6-1 (1.0 g, 5.8 mmol, 1.0 eq) in ethanol (10 mL) was added compound 1-2 (1.5 g, 8.8 mmol, 1.5 eq). The mixture was microwaved at 100° C. for 45 min under Nitrogen atmosphere. TLC analysis of the reaction mixture showed full conversion to the desired product. Then the mixture was filtered and concentrated under reduced pressure. The residue was purified by column chromatography on a silica gel to afford compound 6-2 (400 mg, 23%). TLC (DCM:MeOH, 5:1): Rf (compound 6-1)=1; Rf (compound 6-2)=0.3.

Step 2: To a mixture of compound 6-2 (400 mg, 1.4 mmol, 1.0 eq) in dimethylformamide (5 mL) was added compound 1-4 (201 mg, 1.4 mmol, 1.0 eq), 1-(3-Dimethylaminopropyl)-3-ethylcarbodiimide hydrochloride (349 mg, 1.82 mmol, 1.3 eq), 1-hydroxybenzotriazole (584 mg, 1.82 mmol, 1.3 eq) and triethylamine (541.8 mg, 4.2 mmol, 3.0 eq). The mixture was stirred at room temperature overnight. TLC analysis of the reaction mixture showed full conversion to the desired product. Then the mixture was diluted with water (10 mL), extracted with ethyl acetate (2×10 mL). The combined organic layers were dried over sodium sulfate and concentrated under reduced pressure. The residue was purified by column chromatography (petroleum ether: ethyl acetate, 3:1-1:1) on a silica gel to afford crude compound 6-3 (517 mg, 90%) as a white solid. TLC (petroleum ether: ethyl acetate, 1:1): R_{f} (compound 6-2) =0; R_{f} (compound 6-3)=0.5.

Step 3: A mixture of compound 6-3 (800 mg, 1.95 mmol, 1.0 eq), compound 1-6 (168 mg, 2.15 mmol, 1.1 eq), Xantphos (68 mg, 0.117 mmol, 0.06 eq), Palladium (II) Acetate (22 mg, 0.097 mmol, 0.05 eq) and Tripotassium phosphate (571 mg, 1.1 mmol, 1.1 eq) in dimethylformamide (10 mL) was microwaved at 120° C. for 30 min under Nitrogen atmosphere. TLC analysis of the reaction mixture showed full conversion to the desired product. Then the mixture was cooled to room temperature and filtered. The filtrate was poured into water and extracted with ethyl acetate (3×10 mL). The combined organic layers were washed with water and brine, dried over anhydrous sodium sulfate and concentrated in vacuo. The residue was purified by silica gel chromatography (dichloromethane: methanol=30:1) to give compound 6-4 (480 mg, 51%). TLC (dichloromethane: methanol, 10:1): R_{f} (compound 6-3)=0.7; R_{f} (compound 6-4)=0.4.

Step 4: To a mixture of compound 6-4 (600 mg, 1.47 mmol, 1.0 eq) in i-pranol (5 mL) was added cerium(III) chloride heptahydrate (220 mg, 0.588 mmol, 0.4 eq). The mixture was stirred at 85° C. overnight under oxygen atmosphere. TLC analysis of the reaction mixture showed full conversion to the desired product. Then the mixture was diluted with water (10 mL), extracted with ethyl acetate (2×10 mL). The combined organic layers were dried over sodium sulfate and concentrated under reduced pressure. The residue was purified by Prep-HPLC to afford 6-5 (102 mg, 16%) as a white solid. LCMS: [M+1]: 424; ¹H NMR (CD₃OD, 400 MHz): δ 9.22 (d, J=2.4 Hz, 1H), 8.28-8.26 (m, 1H), 8.02-8.00 (m, 1H), 6.92-6.91 (d, J=6.0 Hz, 2H), 6.81-6.76 (t, J=9.2 Hz, 1H), 4.56 (s, 1H), 4.35 (m, 2H), 4.02 (m, 2H), 2.76-2.72 (m, 2H), 2.29-2.25 (m, 1H), 1.80 (s, 3H) and 1.77 (s, 3H).

Step 5: Similar to the chiral separation procedure for compound 1, compound 6-5 was applied to a Chiralcel OD column to afford compound 6 and 6a.

### Example 7

### (S)-N-(3-chloro-5-fluorobenzyl)-1-(4-(diisopropylphosphoryl)phenyl)-3-hydroxy-2-oxopyrr olidine-3-carboxamide (7)

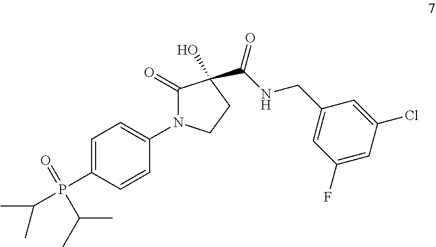 [SMILES: CC(C)P(=O)(c1ccc(N2CC[C@](O)(C(=O)NCc3cc(F)cc(Cl)c3)C2=O)cc1)C(C)C]

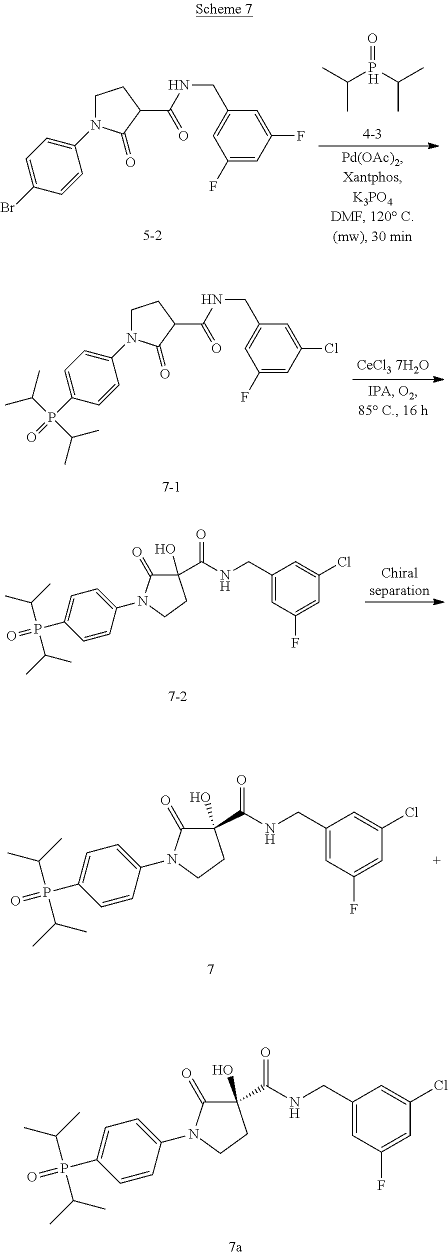

Step 1: To a mixture of compound 5-2 (400 mg, 0.94 mmol) in DMF (3 mL) was added compound 4-3 (84 mg, 1.08 mmol), Pd(OAc)₂ (11 mg, 0.05 mmol), Xantphos (34 mg, 0.06 mmol) and K₃PO₄ (288 mg, 1.08 mmol). The mixture was stirred at 120° C. for 30 min in MW. TLC analysis of the reaction mixture showed full conversion to the desired product. The residue was dried over Na₂SO₄ and concentrated under reduced pressure. The residue was purified by column chromatography on a silica gel eluting by 5% CH₃OH in CH₂Cl₂ to afford compound 7-1 (120 mg, 0.25 mmol, 27%). TLC (DCM:MeOH, 10:1): Rf (compound 7-1)=0.4, Rf (compound 5-2)=0.8.

Step 2: To a mixture of compound 7-1 (120 mg, 0.25 mmol) in IPA (5 mL) was added CeCl₃ 7H₂O (40 mg, 0.11 mmol). The mixture was stirred at 85° C. overnight under O₂. TLC analysis of the reaction mixture showed full conversion to the desired product. The residue was dried over Na₂SO₄ and concentrated under reduced pressure. The residue was purified by prep-HPLC to afford 7-2 (33 mg, 0.07 mmol, 28%) as a white solid. TLC (DCM:MeOH, 10:1): Rf (compound 7-1)=0.5, Rf (7-2)=0.3. LCMS: [M+1]: 495. 1H NMR (400 MHz, CD₃OD) δ7.94 (dd, J=8.7, 2.0 Hz, 2H), 7.74 (t, J=9.0 Hz, 2H), 7.17 (s, 1H), 7.07-6.94 (m, 2H), 4.46 (d, J=15.6 Hz, 1H), 4.36 (d, J=15.6 Hz, 1H), 4.07-3.89 (m, 2H), 2.72 (ddd, J=13.1, 7.6, 3.5 Hz, 1H), 2.44 (td, J=14.5, 7.1 Hz, 2H), 2.25 (dt, J=13.1, 8.1 Hz, 1H), 1.18-1.12 (m, 6H), 1.06-0.98 (m, 6H).

Step 3: Similar to the chiral separation procedure for compound 1 and 1a, compound 7-2 was applied to a Chiralcel OD column to afford compound 7 and 7a.

### Example 8

### (S)-N-(3-chloro-5-fluorobenzyl)-1-(4-(dicyclopropylphosphoryl)phenyl)-3-hydroxy-2-oxopy rrolidine-3-carboxamide (8)

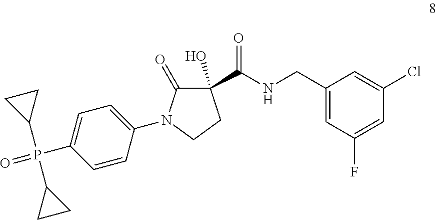 [SMILES: O=C(NCc1cc(F)cc(Cl)c1)[C@@]1(O)CCN(c2ccc(P(=O)(C3CC3)C3CC3)cc2)C1=O]

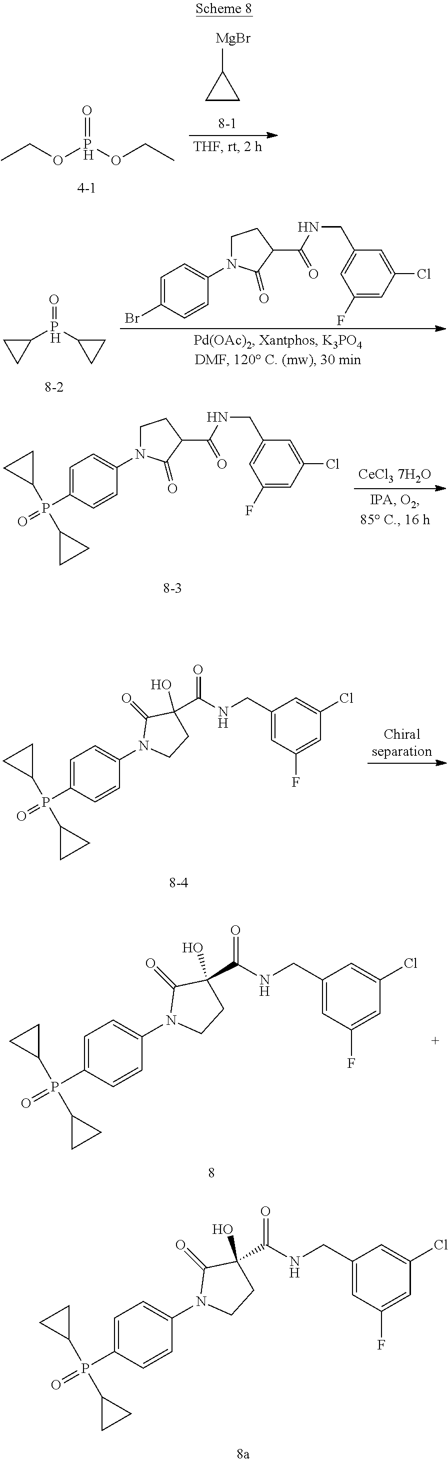

Step 1: A 100 mL RB flask equipped with an addition funnel was evacuated/N₂ filled (3X), then charged with cyclopropylmagnesium chloride 8-1 (1M in THF, 16.5 mL, 3.3 eq), and the solution was cooled to 0° C. under N₂. A solution of diethylphosphite 4-1 (690 mg, 5 mmol, 1.0 eq) in 10 mL THF was added dropwise over 15 minutes. The mixture was stirred at 0° C. for 15 min and at 25° C. for 2 hours. The reaction was cooled to 0° C. 0.1N HCl (15 mL) was added dropwise over 20 mins, then MTBE (25 mL) was added. The upper organic phase was decanted from the gel and saved. To the remaining gel was added 25 mL of CH₂Cl₂, and the mixture was stirred well for 5 minutes. The resulted mixture was then filtered through a celite pad and the pad was washed with CH₂Cl₂. The filtrate phases were separated, and the organic phase was combined, dried (MgSO4), and concentrated in vacuo. The residue was purified on silica gel eluting by 3% CH₃OH in CH₂Cl₂ to afford compound 8-2 (210 mg, 1.62 mmol, 32% yield) as a colorless oil. TLC (DCM:MeOH, 10:1): Rf (compound 8-1)=0.5, Rf (compound 8-2)=0.3. ¹H NMR (400 MHz, CDCl₃) δ 6.55 (d, J=468 Hz, 1H), 1.09-0.62 (m, 10H).

Compound 8 and 8a were prepared using the similar procedure as in compound 2. For compound 8-4, LCMS: [M+1]: 491; ¹H NMR (400 MHz, CDCl₃) δ 7.66 (d, J=7.2 Hz, 3H), 7.53 (d, J=7.2 Hz, 2H), 7.04 (s, 1H), 6.92 (dd, J=22.6, 8.6 Hz, 2H), 4.43 (dd, J=15.1, 6.6 Hz, 1H), 4.32 (dd, J=15.7, 6.0 Hz, 1H), 3.93 (d, J=8.0 Hz, 1H), 3.62 (s, 1H), 2.77-2.68 (m, 1H), 2.28 (d, J=12.7 Hz, 1H), 0.90 (d, J=12.9 Hz, 1OH).

### Example 9

### (S)-N-(3-chloro-5-fluorobenzyl)-1-(4-(dimethylphosphoryl)phenyl)-3-hydroxy-2-oxopyrroli dine-3-carboxamide (9)

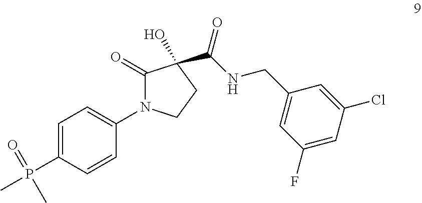 [SMILES: CP(C)(=O)c1ccc(N2CC[C@](O)(C(=O)NCc3cc(F)cc(Cl)c3)C2=O)cc1]

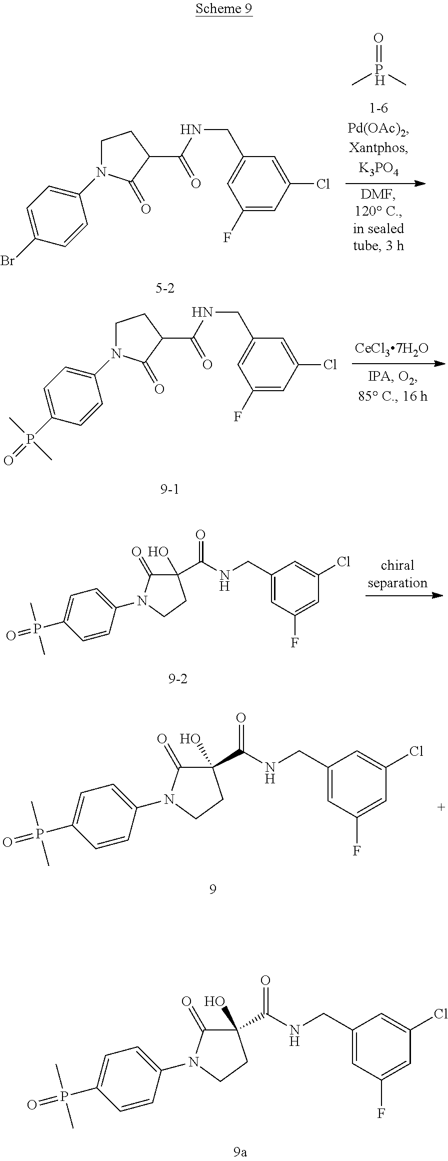

Step 1: The mixture of compound 5-2 (300 mg, 0.705 mmol, 1.0 eq), compound 1-6 (60.51 mg, 0.775 mmol, 1.1 eq), Palladium (II) Acetate (9.49 mg, 0.042 mmol, 0.06 eq), Xantphos (28.55 mg, 0.049 mmol, 0.07 eq) and potassium carbonate (164.51 mg, 0.775 mmol, 1.1 eq) in dimethylformamide (5 mL) was stirred at 120° C. for 3 h in a sealed tube. LCMS analysis of the reaction mixture showed full conversion to the desired product. The mixture was filtered, added water (5 mL), extracted with ethyl acetate (3×5 mL). The combined organic layers were dried over sodium sulfate and concentrated under reduced pressure. The residue was purified by column chromatography on a silica gel (dichloromethane: methanol, 94:6) to afford compound 9-2 (55 mg, 180%) as a yellow solid. 001771 Step 2: To a mixture of compound 9-1 (130 mg, 0.307 mmol, 1.0 eq) in PA (10 mL) was added cerium (III) chloride heptahydrate (45.8 mg, 0.123 mmol, 0.4 eq). The mixture was stirred at 85° C. overnight under oxygen atmosphere. TLC analysis of the reaction mixture showed full conversion to the desired product. The mixture was diluted with water (10 mL), extracted with dichloromethane (2×10 mL). The combined organic layers were dried over sodium sulfate and concentrated under reduced pressure. The residue was purified by prep-TLC (dichloromethane: methanol=10:1) to afford 9-2 (49.4 mg, 37% o) as white solid. TLC (dichloromethane: methanol, 15:1): Rf(compound 9-1)=0.6, Rf (9-2)=0.5. LCMS: [M+1]: 439. ¹H NMR (CD₃OD, 400 MHz): δ 7.96 (m, 2H), 7.94-7.82 (m, 2H), 7.22 (s, 1H), 7.09-7.06 (m, 2H), 4.52-4.38 (dd, J=40.4 Hz, 15.6 Hz, 2H), 4.05-4.00 (m, 2H), 2.76-2.74 (m, 1H), 2.30-2.27 (in, 1H) and 1.82-1.79 (d, J=13.2 Hz, 6H).

Step 3: Similar to the chiral separation procedure for compound 1 and 1a, compound 9-2 was applied to a Chiralcel OD column to afford compound 9 and 9a.

### Example 10

### (S)-1-(4-(dicyclopropylphosphoryl)phenyl)-N-(3,5-difluorobenzyl)-3-hydroxy-2-oxopyrrolid ine-3-carboxamide (10)

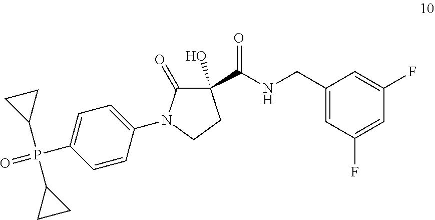 [SMILES: O=C(NCc1cc(F)cc(F)c1)[C@@]1(O)CCN(c2ccc(P(=O)(C3CC3)C3CC3)cc2)C1=O]

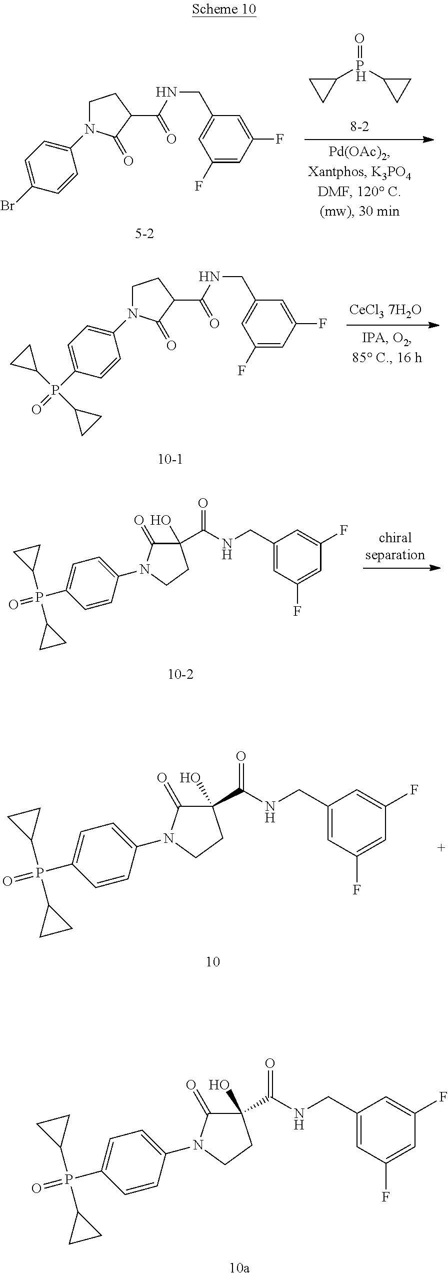

Step 1: The mixture of compound 1-5 (300 mg, 0.70 mmol, 1.0 eq), compound 8-2 (100.1 mg, 0.77 mmol, 1.1 eq), Palladium (II) Acetate (9.49 mg, 0.042 mmol, 0.06 eq), Xantphos (28.6 mg, 0.049 mmol, 0.07 eq) and potassium carbonate (164.5 mg, 0.775 mmol, 1.1 eq) in dimethylformamide (5 mL) was microwaved at 120° C. for 30 min under nitrogen atmosphere. LCMS analysis of the reaction mixture showed full conversion to the desired product. The mixture was filtered, added water (5 mL), extracted with ethyl acetate (3×5 mL). The combined organic layers were dried over sodium sulfate and concentrated under reduced pressure. The residue was purified by column chromatography on a silica gel (dichloromethane: methanol=94:6) to afford compound 10-1 (69.7 mg, 20%) as a yellow solid.

Step 2: To a mixture of compound 10-1 (130 mg, 0.31 mmol, 1.0 eq) in IPA (10 mL) was added cerium (III) chloride heptahydrate (45.8 mg, 0.12 mmol, 0.4 eq). The mixture was stirred at 85° C. overnight under oxygen atmosphere. TLC analysis of the reaction mixture showed full conversion to the desired product. The mixture was diluted with water (10 mL), extracted with dichloromethane (2×10 mL). The combined organic layers were dried over sodium sulfate and concentrated under reduced pressure. The residue was purified by prep-TLC (dichloromethane: methanol, 10:1) to afford 10-2 (48.9 mg, 33%) as a white solid. TLC (dichloromethane: methanol, 15:1): Rf (compound 10-1)=0.6, Rf (10-2)=0.5. LCMS: [M+1]: 475. ¹H NMR (DMSO, 400 MHz): δ 8.45-8.31 (t, J=6.4 Hz, 1H), 7.83-7.76 (m, 4H), 7.04-7.01 (m, 1H), 6.97-6.94 (m, 2H), 6.83 (s, 1H), 4.39-4.34 (dd, J=15.6 Hz, 6.8 Hz, 1H), 4.25-4.19 (dd, J=15.6 Hz, 6.0 Hz, 1H), 3.89-3.86 (t, J=6.8 Hz, 1H), 2.56-2.46 (m, 1H), 2.16-2.11 (m, 1H), 1.21-1.15 (m, 2H), 0.80-0.67 (m, 6H) and 0.59-0.53 (m, 2H).

Step 3: Similar to the chiral separation procedure for compound 1 and 1a, compound 10-2 was applied to a Chiralcel OD column to afford compound 10 and 10a.

### Example 11

### (S)-N-(3,5-difluorobenzyl)-3-hydroxy-2-oxo-1-(4-(trimethylsilyl)phenyl)pyrrolidine-3-carbo xamide (11)

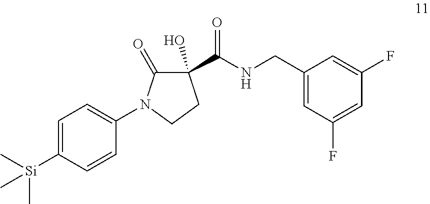 [SMILES: C[Si](C)(C)c1ccc(N2CC[C@](O)(C(=O)NCc3cc(F)cc(F)c3)C2=O)cc1]

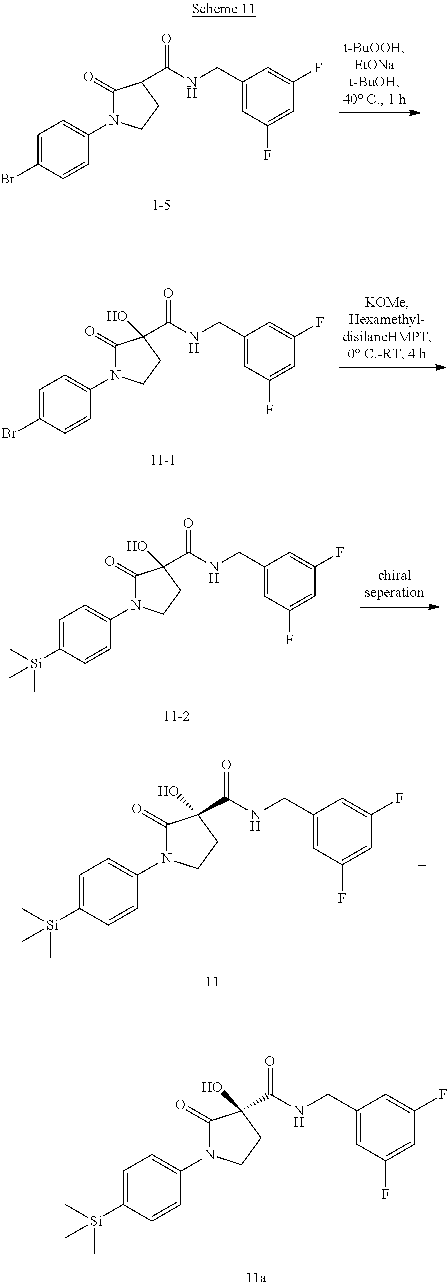 [SMILES: O=C(NCc1cc(F)cc(F)c1)C1CCN(c2ccc(Br)cc2)C1=O ; O=C(NCc1cc(F)cc(F)c1)C1(O)CCN(c2ccc(Br)cc2)C1=O ; C[Si](C)(C)c1ccc(N2CCC(O)(C(=O)NCc3cc(F)cc(F)c3)C2=O)cc1 ; C[Si](C)(C)c1ccc(N2CC[C@](O)(C(=O)NCc3cc(F)cc(F)c3)C2=O)cc1 ; C[Si](C)(C)c1ccc(N2CC[C@@](O)(C(=O)NCc3cc(F)cc(F)c3)C2=O)cc1]

Step 1: To a mixture of compound 1-5 (320 mg, 0.781 mmol) in t-BuOH (10 mL) at room temperature was added slowly EtONa (21% in EtOH, 500 mg, 1.54 mmol) and t-BuOOH (70% in H₂O, 200 mg, 1.55 mmol). Then the mixture was stirred at 40° C. for 1 h under nitrogen atmosphere. After the completion of the reaction, the mixture was quenched with saturated Na₂SO₃ solution and extracted with DCM (20 mL×2). The organic layers was washed with brine, dried over anhydrous Na₂SO₄, filtered and concentrated. The residue was purified by silical gel column (eluted with 5% MeOH in DCM) to afford the title compound 11-1 (145 mg, yield 43%) as a white solid. TLC (DCM:MeOH/20:1): Rf (compound 1-5)=0.65; Rf (compound 11-1)=0.55.

Step 2: To a solution of compound 11-1 (50 mg, 0.117 mmol) in HMPT (2 mL) was added KOMe (16 mg, 0.228 mmol) at 0° C. under nitrogen atmosphere. After stirring for 30 min, hexamethyldisilane (74 mg, 0.467 mmol) was added dropwise, and the mixture was stirred at room temperature for additional 4 h. After the completion of the reaction, the mixture was quenched with saturated NH₄Cl solution and extracted with DCM (10 mL×2). The organic layers was washed with brine, dried over anhydrous Na₂SO₄, filtered and concentrated. The residue was purified by silical gel column (eluted with 5% MeOH in DCM) to afford 11-2 (8.5 mg, yield 17%) as a white solid. ¹H NMR (500 MHz, Chloroform-d) δ 7.62 (d, J=8.1 Hz, 2H), 7.55 (d, J=8.4 Hz, 2H), 7.40 (d, J=6.4 Hz, 1H), 6.84-6.78 (m, 2H), 6.74-6.68 (m, 1H), 4.43 (qd, J=15.4, 6.2 Hz, 2H), 4.21 (td, J=9.1, 6.9 Hz, 1H), 4.13 (s, 1H), 3.86 (t, J=9.0 Hz, 1H), 2.80 (ddd, J=12.8, 7.0, 1.5 Hz, 1H), 2.31 (dt, J=12.8, 9.2 Hz, 1H), 0.28 (s, 9H). LCMS: m/z calculated for C₂₁H₂₄F₂N₂O₃Si: 418.52; found: 419.3 [M+H]⁺.

Step 3: Similar to the chiral separation procedure for compound 1 and 1a, compound 11-2 was applied to a Chiralcel OD column to afford compound 11 and 11a.

### Example 12

### (S)-N-(3,5-difluorobenzyl)-3-hydroxy-2-oxo-1-(spiro[cyclopropane-1,3′-indoline]-5′-yl)pyrr olidine-3-carboxamide (12)

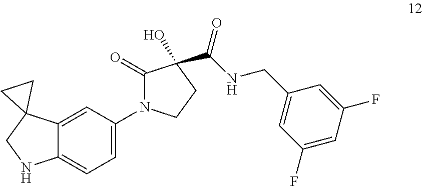 [SMILES: O=C(NCc1cc(F)cc(F)c1)[C@@]1(O)CCN(c2ccc3c(c2)C2(CC2)CN3)C1=O]

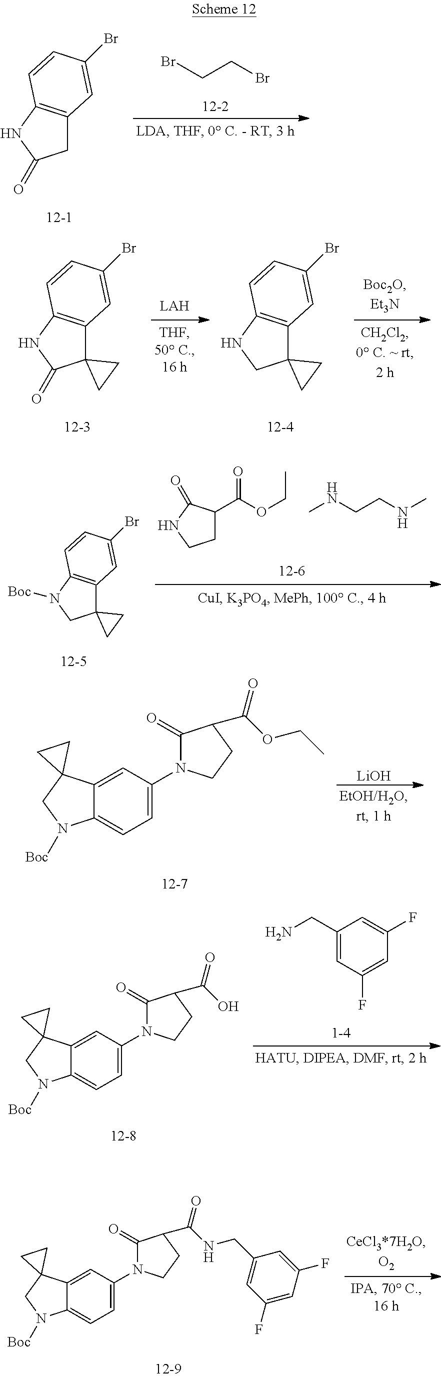

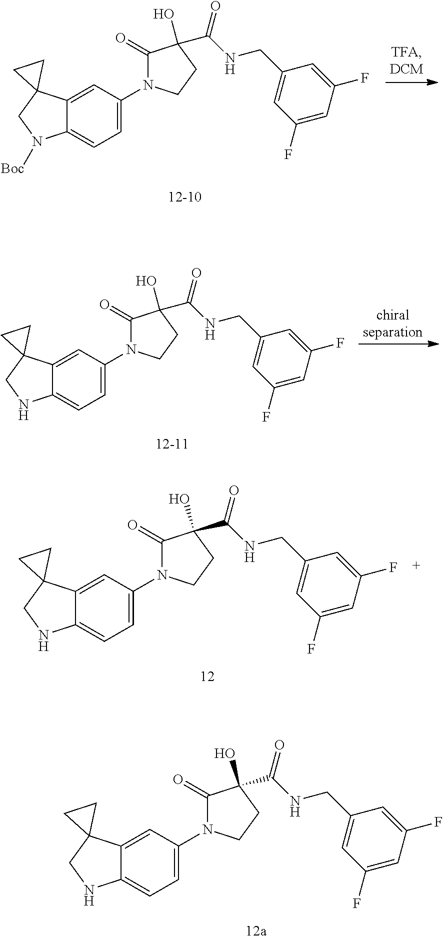 [SMILES: CC(C)(C)OC(=O)N1CC2(CC2)c2cc(N3CCC(O)(C(=O)NCc4cc(F)cc(F)c4)C3=O)ccc21 ; O=C(NCc1cc(F)cc(F)c1)C1(O)CCN(c2ccc3c(c2)C2(CC2)CN3)C1=O ; O=C(NCc1cc(F)cc(F)c1)[C@@]1(O)CCN(c2ccc3c(c2)C2(CC2)CN3)C1=O ; O=C(NCc1cc(F)cc(F)c1)[C@]1(O)CCN(c2ccc3c(c2)C2(CC2)CN3)C1=O]

Step 1: To a solution of compound 12-1 (10 g, 47.4 mmol) in THF (100 mL) was added dropwise LDA (2M, 95 mL, 4 eq.) at 0° C. The reaction mixture stirred for 0.5h and then added 1,2-dibromoethane (12-2) (11.7 g, 62.25 mmol) at 0° C. The reaction was warmed to room temperature and stirred for 3 h. The reaction was quenched with water. The mixture was extracted with ethyl acetate (3*100 mL). The combined organic layers were washed with water and brine, dried over sodium sulfate, filtered, and concentrated by rotary evaporation. The resulting residue was purified by column chromatography (30% EtOAc in Petrol) to give the compound 12-3 (6 g) as a light red solid. TLC (PE: EA=1:3): Rf (compound 12-1)=0.3, Rf (compound 12-3)=0.4. LCMS [M+1]: 238.10.

Step 2: LAH(2.5 M, 34 mL, 5 eq) was added to a solution of compound 12-3 (4 g, 1 eq) in THE (40 mL) at 0° C., then the mixture was stirred at 50° C. for 16 hrs. LCMS showed the reaction convention was completely and the mixture was quenched with H₂O (3.3 mL), 3.3 mL NaOH aq.(10%), 9.9 mL H₂O and filtered. The filtrate was extracted with EtOAc (50 mL *2). The combined organic layer was dried over anhydrous Na₂SO₄, concentrated and purified by column chromatography (30% EtOAc in Petrol) to afford compound 12-4 (1.3 g). LCMS [M+1]⁺: 224.10;

Step 3: Compound 12-4 (3.1 g, 1 eq.) in DCM (40 mL) was added Boc₂O (6.0 g, 2 eq), TEA (4.1 g, 3 eq) at 0° C., then the mixture was stirred at RT for 2 hrs. LCMS showed the reaction convention was completely, the mixture was concentrated and purified by column chromatography (5% EtOAc in Petrol) to afford compound 12-5 (2.1 g). LCMS: [M+23]⁺ 346.10; Rf (4, PE: EA=5:1)=0.8.

Step 4: To a solution of compound 12-5 (200 mg, 0.62 mmol) in MePh (3 mL) was added ethyl 2-oxopyrrolidine-3-carboxylate 12-6 (149 mg, 0.95 mmol), CuI (23 mg, 0.12 mmol), DMEDA (10.4 mg, 0.12 mmol) and K₃PO₄ (327 mg, 1.55 mmol) under N₂. The reaction mixture was stirred for 4 h at 110° C. The reaction mixture was quenched with H₂O and extracted with ethyl acetate. The combined organics were washed with water and brine, dried over sodium sulfate, filtered, and concentrated by rotary evaporation. The resulting residue was purified by column chromatography (25% EtOAc in petroleum ether) to give the compound 12-7 (90 mg) as a yellow solid. LCMS: [M+23]⁺423.10;

Step 5: Compound 12-7 (90 mg, 0.23 mmol) in EtOH/H₂O (1:1, 3 mL) in an ice bath was added LiGH (11 mg, 0.46 mmol). The reaction mixture was stirred for 1 h at room temperature. The combined organics were concentrated by rotary evaporation to give the compound 12-8 (70 mg).

Step 6: To a solution of Compound 12-8 (70 mg, 0.18 mmol) in DMF (3 mL) was added compound 1-4 (31 mg, 0.22 mmol), HATU (102.6 mg, 0.27 mmol) and DIEA (74.5 mg, 0.56 mmol). The reaction mixture was stirred at room temperature for 2h. The reaction was quenched with water. The mixture was extracted with ethyl acetate (3*20 mL). The combined organic layers were washed with water and brine, dried over sodium sulfate and concentrated by rotary evaporation. The resulting residue was purified by column chromatography (40% EtOAc in petroleum ether) to give the compound 12-9 (70 mg) as a white solid. LCMS: [M+1]+497.10;

Step 7: Compound 12-9 (70 mg, 0.14 mmol) in IPA (3 mL) was added CeCl₃*7H₂O (31 mg, 0.08 mmol) and the mixture was stirred at 70° C. for 18 hrs under a 02 atmosphere. TLC showed full convention. The mixture was quenched with water. The mixture was extracted with ethyl acetate (3*5 mL). The combined organic layers were washed with water, dried over sodium sulfate and concentrated by rotary evaporation in vacuo. The resulting residue purified by column chromatography (50% EtOAc in petroleum ether) to give the compound 12-10 (40 mg). LCMS: [M+1]+514.2;

Step 8: To a solution of 12-10 (40 mg, 0.1 mmol) in DCM (2 mL) was added TFA (1 mL) and the mixture was stirred at RT for 3 hrs. The mixture was concentrated in vacuo and purified by prep-HPLC to afford compound 12-11 (21 mg) as an off-white solid. ¹H NMR (400 MHz, CD₃OD) δ8.80 (s, 1H), 7.59 (d, J=8.6 Hz, 1H), 7.43-7.26 (m, 2H), 6.91 (d, J=6.6 Hz, 2H), 6.78 (t, J=9.1 Hz, 1H), 4.48 (dd, J=15.7, 6.6 Hz, 1H), 4.35 (dd, J=15.7, 5.8 Hz, 1H), 4.02-3.84 (m, 2H), 3.78 (s, 2H), 2.74-2.62 (m, 1H), 2.23 (dt, J=13.1, 8.0 Hz, 1H), 1.25-1.17 (m, 4H). LCMS: [M+1]⁺414.25;

Step 9: Similar to the chiral separation procedure for compound 1 and 1a, compound 12-11 was applied to a Chiralcel OD column to afford compound 12 and 12a.

### Example 13

### (S)-N-(3-chloro-5-fluorobenzyl)-3-hydroxy-2-oxo-1-(2′-oxospiro[cyclopropane-1,3′-indoline]-5′-yl)pyrrolidine-3-carboxamide (13)

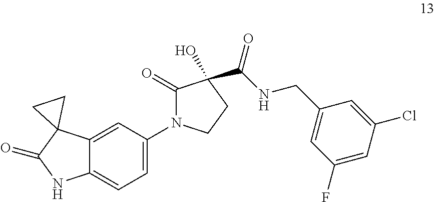 [SMILES: O=C1Nc2ccc(N3CC[C@](O)(C(=O)NCc4cc(F)cc(Cl)c4)C3=O)cc2C12CC2]

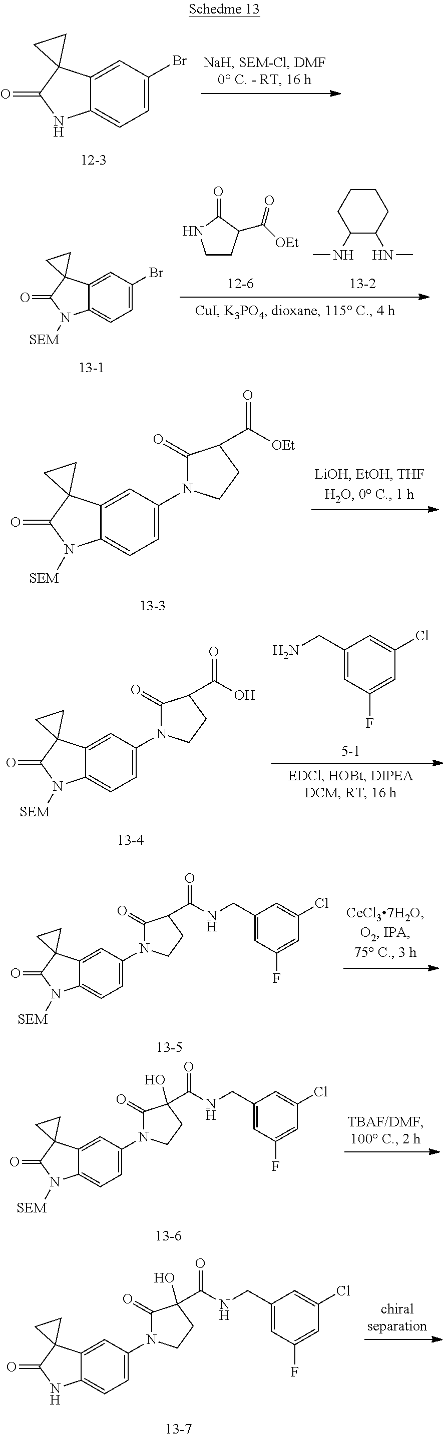

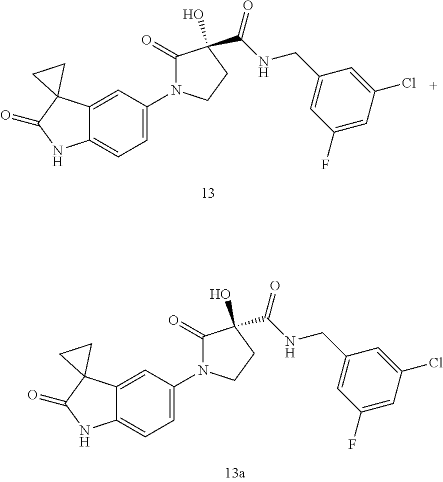 [SMILES: O=C1Nc2ccc(N3CC[C@](O)(C(=O)NCc4cc(F)cc(Cl)c4)C3=O)cc2C12CC2 ; O=C1Nc2ccc(N3CC[C@@](O)(C(=O)NCc4cc(F)cc(Cl)c4)C3=O)cc2C12CC2]

Step 1: To a mixture of compound 12-3 (17.8 g, 74.76 mmol) in anhydrous DMF (80 mL) was added NaH (60% in mineral oil, 5.98 g, 149 mmol) in several portions at 0° C. under nitrogen. After stirring at 0° C. for 30 min, SEM-Cl (18.7 g, 112 mmol) was added to the mixture. The reaction was stirred at room temperature for 16 h. After the completion of the reaction, ice was added to quench the reaction. The resulted mixture was partitioned between EtOAc (300 mL) and water (100 mL). The organic layer was washed with brine (200 mL), dried over anhydrous Na₂SO₄ and concentrated. The crude product was purified by silica gel column (eluted with 5% EtOAc in petroleum ether) to afford the title compound 13-1 (22.8 g, yield 82%) as a white crystal. TLC (Petroleum ether:EtOAc/20:1): R_{f}(12-3)=0.1; R_{f}(13-1)=0.45.

Step 2: While under nitrogen, a mixture of compound 13-1 (9.0 g, 24.43 mmol), compound 12-6 (5 g, 31.82 mmol), compound 13-2 (1.73 g, 12.16 mmol), CuI (2.32 g, 12.18) and K₃PO₄ (15.6 g, 73.49 mmol) in dioxane (90 mL) was stirred at 115° C. for 4 h. The mixture was filtered over celite, diluted with water (100 mL) and extracted with EtOAc (200 mL×2). The organic layers were washed with brine, dried over anhydrous Na₂SO₄, filtered and concentrated. The residue was purified by silica gel column (eluted with 30% EtOAc in petroleum ether) to afford title compound 13-3 (6.6 g, yield 60%) as a light yellow oil. TLC (Petroleum ether:EtOAc/2:1): R_{f} (compound 13-1)=0.8; R_{f} (compound 13-3)=0.35.

Step 3: To a solution of compound 13-3 (6.6 g, 14.86 mmol) in EtOH (20 mL), THE (10 mL) and H₂O (20 mL) was added LiOH·H₂O (1.25 g, 29.79 mmol) at 0° C. After the mixture was stirred at room temperature for 1 h, the mixture was concentrated to get rid of organic solvents. The left aqueous solution was adjusted to pH=2 and extracted with EtOAc (50 mL×2). The organic layers were washed with brine, dried over anhydrous Na₂SO₄, filtered and concentrated to afford the title compound 13-4 (5.8 g, yield 93%) as a light yellow oil, which was used directly at the next step. TLC (Petroleum ether:EtOAc/2:1): R_{f} (compound 13-3)=0.35; R_{f}(compound 13-4:)=0.1.

Step 4: A mixture of compound 13-4 (3.0 g, 7.2 mmol), compound 5-1 (1.72 g, 10.78 mmol), HOBt (970 mg, 7.18 mmol), DIPEA (2.32 g, 17.98 mmol) and EDCI (2.47 g, 12.93 mmol) in anhydrous DCM (30 mL) was stirred at room temperature for 16 h. After the completion of the reaction, the mixture was diluted by EtOAc (100 mL) and washed with water, 1 N HCl and brine. The organic layer was dried over anhydrous Na₂SO₄, filtered and concentrated. The residue was purified by silica gel column (eluted with 30% EtOAc in petroleum ether) to afford the title compound 13-5 (3.6 g, yield 90%) as a foam. TLC (Petroleum ether:EtOAc/2:1): R_{f} (compound 13-4)=0.1; R_{f} (compound 13-5)=0.4.

Step 5: A mixture of compound 13-5 (5.14 g, 9.21 mmol) and CeCl₃.7H₂O (1.37 g, 3.67 mmol) in isopropanol (50 mL) was stirred at 75° C. under oxygen atmosphere for 3 h. After the completion of the reaction, the mixture was concentrated. The residue was purified by silica gel column (eluted with 5% MeOH in DCM) to afford the title compound 13-6 (3.54 g, yield 64%) as a white solid. TLC (DCM:MeOH/20:1): R_{f} (compound 13-5)=0.46; R_{f} (compound 13-6)=0.35.

Step 6: A solution of compound 13-6 (3.5 g, 6.09 mmol) and TBAF (1 M THE solution, 30 mL, 30 mmol) in DMF (30 mL) was stirred at 100° C. under nitrogen atmosphere for 2 h. After the completion of the reaction, the mixture was cooled to room temperature and diluted by EtOAc (100 mL) and water (100 mL). The mixture was separated, the organic layer was washed by 1 N HCl, saturated NaHCO₃ and brine, dried over anhydrous Na₂SO₄, filtered and concentrated. The residue was purified by silica gel column (eluted with 5% MeOH in DCM) to afford 13-7 (1.2 g, yield 44%) as a white powder. ¹H NMR (400 MHz, DMSO-d6) δ 10.60 (s, 1H), 8.73 (t, J=6.4 Hz, 1H), 7.49 (dd, J=8.4, 2.2 Hz, 1H), 7.28 (dt, J=8.9, 2.2 Hz, 1H), 7.24-7.15 (m, 2H), 7.10 (dt, J=9.6, 1.8 Hz, 1H), 6.91 (d, J=8.3 Hz, 1H), 6.72 (s, 1H), 4.38 (dd, J=15.8, 6.7 Hz, 1H), 4.24 (dd, J=15.7, 6.1 Hz, 1H), 3.80 (dd, J=7.9, 5.6 Hz, 2H), 2.57 (dt, J=12.1, 5.9 Hz, 1H), 2.09 (dt, J=13.0, 7.5 Hz, 1H), 1.55 (t, J=3.3 Hz, 2H), 1.48 (q, J=4.1, 3.4 Hz, 2H). LCMS: m/z calculated for C₂₂H₁₉C1FN₃O₄: 443.86; found: 444.16. [M+H]⁺. TLC (DCM:MeOH/20:1): R_{f} (compound 13-6) =0.35, R_{f} (13-7)=0.25.

Step 7: Similar to the chiral separation procedure for compound 1 and 1a, compound 13-7 was applied to a Chiralcel OD column to afford compound 13 and 13a.

### Example 14

### (S)-N-(3-chloro-5-fluorobenzyl)-3-hydroxy-2-oxo-1-(2′-oxospiro[cyclobutane-1,3′-indoline]-5′-yl)pyrrolidine-3-carboxamide (14)

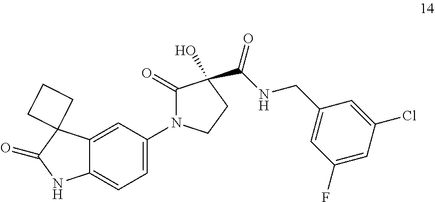 [SMILES: O=C1Nc2ccc(N3CC[C@](O)(C(=O)NCc4cc(F)cc(Cl)c4)C3=O)cc2C12CCC2]

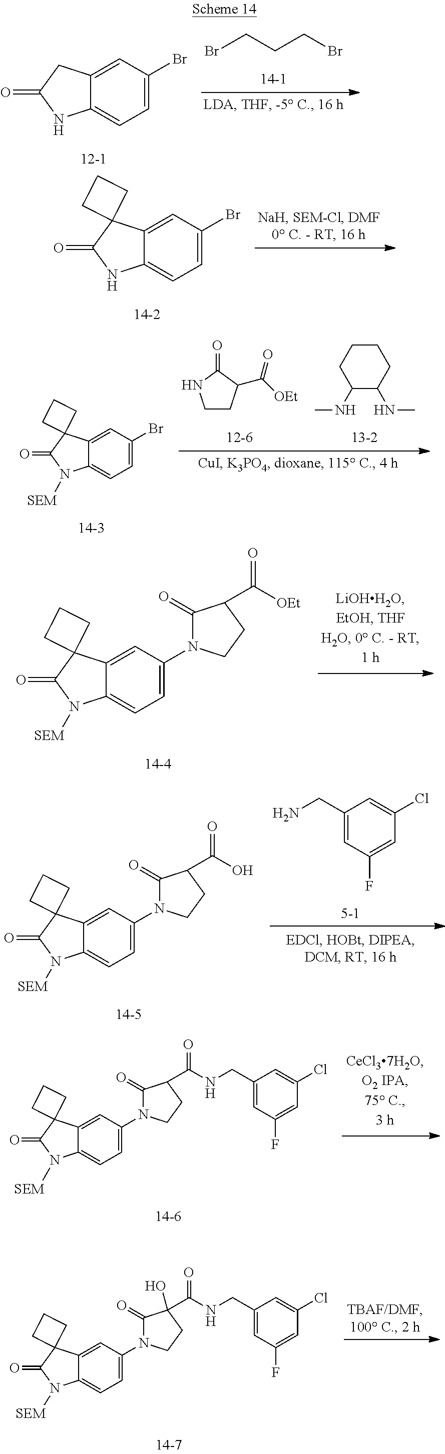

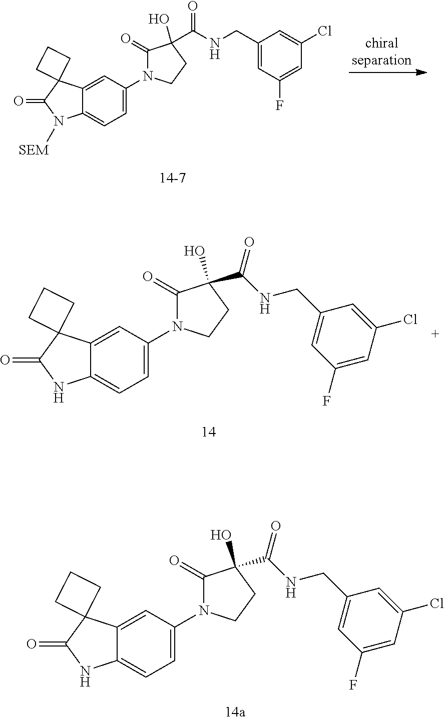 [SMILES: C[Si](C)(C)CCOCN1C(=O)C2(CCC2)c2cc(N3CCC(O)(C(=O)NCc4cc(F)cc(Cl)c4)C3=O)ccc21 ; O=C1Nc2ccc(N3CC[C@](O)(C(=O)NCc4cc(F)cc(Cl)c4)C3=O)cc2C12CCC2 ; O=C1Nc2ccc(N3CC[C@@](O)(C(=O)NCc4cc(F)cc(Cl)c4)C3=O)cc2C12CCC2]

Step 1: To a solution of compound 12-1 (5 g, 23.6 mmol) in anhydrous THF (50 mL) at −5° C. was added dropwise LDA (2 M in THF solution, 48 mL, 96 mmol) under nitrogen atmosphere. After stirring at −5° C. for 30 min, compound 14-1 (14.3 g, 70.8 mmol) was added in one portion. Then the mixture was stirred at room temperature for 16 h. After the completion of the reaction, water (150 mL) was added slowly to quench the reaction and the mixture was extracted with EtOAc (200 mL×2). The organic layers were washed with brine, dried by anhydrous Na₂SO₄, filtered and concentrated. The residue was triturated with EtOAc to afford the title compound 14-2 (2.6 g, yield 43%) as a pink solid. TLC (Petroleum ether:EtOAc/5:1): R_{f}(compound 12-1)=0.2; R_{f} (compound 14-2)=0.3.

Step 2: To a mixture of compound 14-2 (2.4 g, 9.50 mmol) in anhydrous DMF (20 mL) was added NaH (60% in mineral oil, 759 mg, 18.98 mmol) in several portions at 0° C. under nitrogen. After stirring at for 0° C. for 30 min, SEM-Cl (2.37 g, 14.21 mmol) was added to the mixture. The reaction mixture was stirred at room temperature for 16 h. After the completion of the reaction, ice was added to quench the reaction. The resulted mixture was partitioned between EtOAc (150 mL) and water (100 mL). The organic layer was washed with brine (100 mL), dried over anhydrous Na₂SO₄ and concentrated. The crude product was purified by silica gel column (eluted with 5% EtOAc in petroleum ether) to afford the title compound 14-3 (2.83 g, yield 77%) as a colorless oil. TLC (Petroleum ether:EtOAc/20:1): R_{f} (compound 14-2)=0.05; R_{f} (compound 14-3)=0.5.

Step 3: While under nitrogen, a mixture of compound 14-3 (1.85 g, 4.83 mmol), compound 12-6 (1.0 g, 6.36 mmol), compound 13-2 (348 mg, 2.44 mmol), CuI (466 mg, 2.45 mmol) and K₃PO₄ (15.6 g, 14.70 mmol) in dioxane (20 mL) was stirred at 115° C. for 4 h. After the completion of the reaction, the mixture was filtered over celite. The filtrate was diluted with water (50 mL) and extracted with EtOAc (50 mL×2). The organic layers were washed with water and brine, dried over anhydrous Na₂SO₄, filtered and concentrated. The residue was purified by silica gel column (eluted with 30% EtOAc in petroleum ether) to afford the title compound 14-4 (1.03 g, yield 46%) as a light yellow oil. TLC (Petroleum ether:EtOAc/5:1): R_{f} (compound 14-3)=0.8; R_{f}(compound 14-4)=0.2

Step 4: To a solution of compound 14-4 (1.03 g, 2.24 mmol) in EtOH (4 mL), THE (2 mL) and H₂O (4 mL) was added LiOH·H₂O (188 mg, 4.48 mmol) at 0° C. After the reaction was stirred at room temperature for 1 h, the mixture was concentrated to get rid of organic solvents. The left aqueous solution was adjusted to pH=2 and extracted with EtOAc (50 mL×2). The organic layers were washed with brine, dried over anhydrous Na₂SO₄, filtered and concentrated to afford the title compound 14-5 (890 mg, yield 92%) as a light yellow oil, which was used directly at the next step. TLC (Petroleum ether:EtOAc/2:1): R_{f} (compound 14-4)=0.48; R_{f} (compound 14-5)=0.1.

Step 5: A mixture of compound 14-5 (890 mg, 2.06 mmol), compound 5-1 (530 mg, 3.32 mmol), HOBt (300 mg, 2.22 mmol), DIPEA (722 mg, 5.60 mmol) and EDCI (770 mg, 4.03 mmol) in anhydrous DCM (10 mL) was stirred at room temperature for 16 h. After the completion of the reaction, the mixture was diluted by EtOAc (50 mL) and washed with water, 1 N HCl and brine. The organic layer was dried over anhydrous Na₂SO₄, filtered and concentrated. The residue was purified by silica gel column (eluted with 30% EtOAc in petroleum ether) to afford the title compound 14-6 (890 mg, yield 75%) as a foam. TLC (Petroleum ether:EtOAc/2:1): R_{f} (compound 5-1)=0.1; R_{f} (compound 14-6)=0.4.

Step 6: A mixture of compound 14-6 (500 mg, 0.873 mmol) and CeCl₃.7H₂O (160 mg, 0.430 mmol) in isopropanol (10 mL) was stirred at 75° C. under oxygen atmosphere for 3 h. After the completion of the reaction, the mixture was concentrated. The residue was purified by silica gel column (eluted with 5% MeOH in DCM) to afford compound 14-7 (400 mg, yield 78%) as a pale-yellow oil. TLC (DCM:MeOH/20:1): R_{f} (compound 14-6)=0.75; R_{f} (compound 14-7)=0.55.

Step 7: A solution of compound 14-7 (400 mg, 0.680 mmol) and TBAF (1 M THE solution, 4 mL, 4 mmol) in DMF (5 mL) was stirred at 100° C. under nitrogen atmosphere for 2 h. After the completion of the reaction, the mixture was cooled to room temperature and diluted by EtOAc (50 mL) and water (50 mL). The mixture was separated, and the organic layer was washed by 1 N HCl, saturated NaHCO₃ and brine, dried over anhydrous Na₂SO₄, filtered and concentrated. The residue was purified by silica gel column (eluted with 5% MeOH in DCM) to afford 14-8 (70 mg, yield 22%) as a white powder. ¹H NMR (400 MHz, Chloroform-d) δ 8.18 (s, 1H), 7.78 (s, 1H), 7.56 (s, 1H), 7.22 (s, 1H), 7.06 (s, 1H), 6.98-6.86 (m, 2H), 6.74 (d, J=8.0 Hz, 1H), 4.80 (s, 1H), 4.42 (dd, J=6.5, 3.3 Hz, 2H), 4.16 (d, J=8.8 Hz, 1H), 3.84 (s, 1H), 2.61 (s, 2H), 2.33 (s, 4H), 1.65 (s, 2H). LCMS: m/z calculated for C₂₃H₂₁ClFN₃O₄:457.89; found: 458.29 [M+H]⁺. TLC (DCM:MeOH/20:1): R_{f} (compound 14-7)=0.55; R_{f} (14-8)=0.45.

Step 8: Similar to the chiral separation procedure for compound 1 and 1a, compound 14-8 was applied to a Chiralcel OD column to afford compound 14 and 14a.

### Example 15

### (S)-N-(3,5-difluorobenzyl)-3-hydroxy-2-oxo-1-(2′-oxospiro[cyclopentane-1,3′-indoline]-5′-yl )pyrrolidine-3-carboxamide (15)

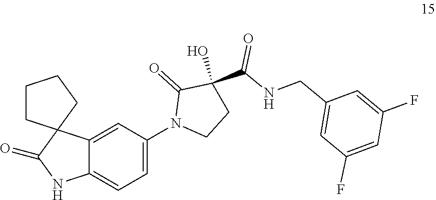 [SMILES: O=C1Nc2ccc(N3CC[C@](O)(C(=O)NCc4cc(F)cc(F)c4)C3=O)cc2C12CCCC2]

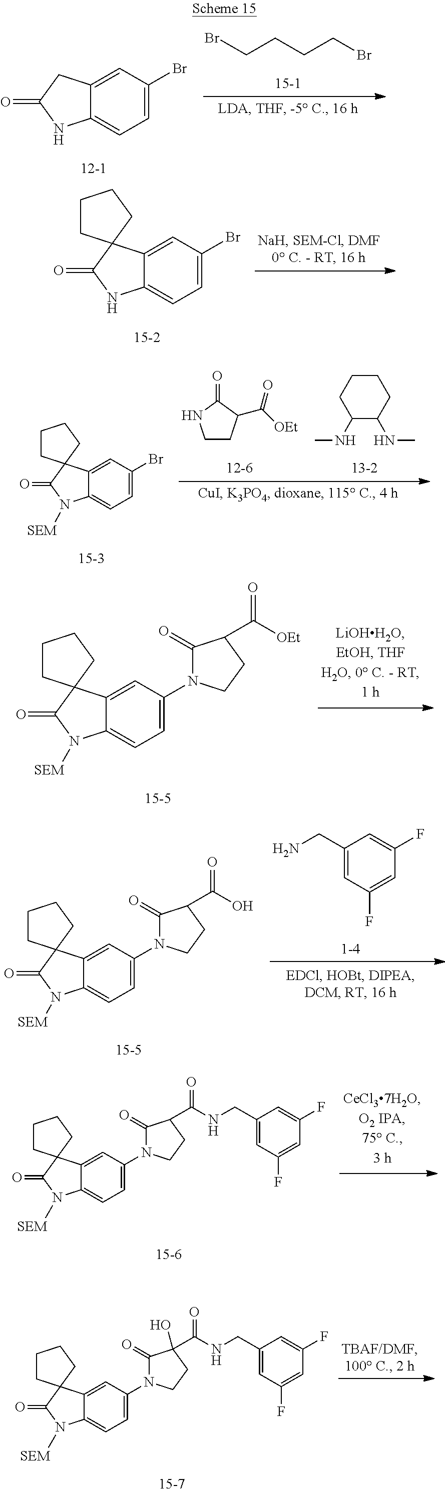

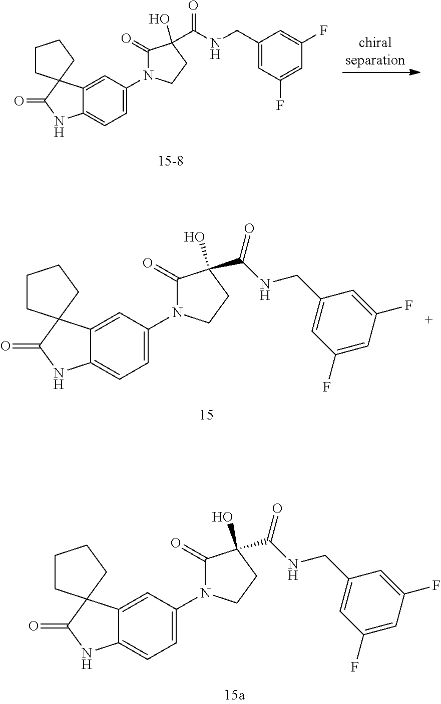 [SMILES: O=C(NCc1cc(F)cc(F)c1)C1(O)CCN(c2ccc3c(c2)C2(CCCC2)C(=O)N3)C1=O ; O=C1Nc2ccc(N3CC[C@](O)(C(=O)NCc4cc(F)cc(F)c4)C3=O)cc2C12CCCC2 ; O=C1Nc2ccc(N3CC[C@@](O)(C(=O)NCc4cc(F)cc(F)c4)C3=O)cc2C12CCCC2]

Step 1: To a solution of compound 12-1 (5 g, 23.6 mmol) in anhydrous THF (50 mL) at −5° C. was added dropwise LDA (2 M in THF solution, 48 mL, 96 mmol) under nitrogen atmosphere. After stirring at −5° C. for 30 min, compound 15-1 (15.3 g, 70.8 mmol) was added in one portion. Then the mixture was stirred at room temperature for 16 h. After the completion of the reaction, water (150 mL) was added slowly to quench the reaction and the mixture was extracted with EtOAc (200 mL×2). The organic layers were washed with brine, dried by anhydrous Na₂SO₄, filtered and concentrated. The residue was triturated with EtOAc to afford compound 15-2 (3.4 g, yield 54%) as a pink solid. TLC (Petroleum ether:EtOAc/5:1):R_{f} (compound 12-1)=0.2; R_{f} (compound 15-2)=0.25.

Step 2: To a mixture of compound 15-2 (3.2 g, 12.02 mmol) in anhydrous DMF (30 mL) was added NaH (60% in mineral oil, 960 mg, 24.00 mmol) in several portions at 0° C. under nitrogen. After stirring at for 0° C. for 30 min, SEM-Cl (3.00 g, 18.00 mmol) was added to the mixture. The reaction was stirred at room temperature for 16 h. After the completion of the reaction, ice was added to quench the reaction. The resulted mixture was partitioned between EtOAc (150 mL) and water (100 mL). The organic layer was washed with brine (100 mL), dried over anhydrous Na₂SO₄ and concentrated. The crude product was purified by silica gel column (eluted with 5% EtOAc in petroleum ether) to afford the title compound 15-3 (2.8 g, yield 58%) as a colorless oil. TLC (Petroleum ether:EtOAc/20:1): R_{f} (compound 15-2)=0.1; R_{f} (compound 15-3)=0.45.

Step 3: While under nitrogen, a mixture of compound 15-3 (1.95 g, 4.92 mmol), compound 12-6 (1.0 g, 6.36 mmol), compound 13-2 (348 mg, 2.45 mmol), CuI (466 mg, 2.45) and K₃PO₄ (3.12 g, 14.7 mmol) in dioxane (20 mL) was stirred at 115° C. for 4 h. After the completion of the reaction, the mixture was filtered over celite. The filtrate was diluted with water (50 mL) and extracted with EtOAc (50 mL×2). The organic layers were washed with water and brine, dried over anhydrous Na₂SO₄, filtered and concentrated. The residue was purified by silica gel column (eluted with 30% EtOAc in petroleum ether) to afford the title compound 15-4 (1.23 g, yield 52%) as a light yellow oil. TLC (Petroleum ether:EtOAc/5:1): R_{f} (compound 15-3)=0.8; R_{f} (compound 15-4)=0.15.

Step 4: To a solution of compound 15-4 (1.23 g, 2.60 mmol) in EtOH (4 mL), THE (2 mL) and H₂O (4 mL) was added LiOH·H₂O (218 mg, 5.20 mmol) at 0° C. After the reaction was stirred at room temperature for 1 h, the mixture was concentrated to get rid of organic solvents. The left aqueous solution was adjusted to pH=2 and extracted with EtOAc (50 mL×2). The organic layers were washed with brine, dried over anhydrous Na₂SO₄, filtered and concentrated to afford the title compound 15-5 (1.0 g, yield 86%) as a light yellow oil, which was used directly at the next step. TLC (Petroleum ether:EtOAc/2:1): R_{f} (compound 15-4)=0.65; R_{f} (compound 15-5) =0.1.

Step 5: A mixture of compound 15-5 (1.0 g, 2.25 mmol), compound 1-4 (560 mg, 3.91 mmol), HOBt (350 mg, 2.59 mmol), DIPEA (725 mg, 5.62 mmol) and EDCI (900 mg, 4.71 mmol) in anhydrous DCM (10 mL) was stirred at room temperature for 16 h. After the completion of the reaction, the mixture was diluted by EtOAc (50 mL) and washed with water, 1 N HCl and brine. The organic layer was dried over anhydrous Na₂SO₄, filtered and concentrated. The residue was purified by silica gel column (eluted with 30% EtOAc in petroleum ether) to afford the title compound 15-6 (1.0 g, yield 78%) as a pale-yellow foam. TLC (Petroleum ether:EtOAc/2:1): R_{f}(compound 1-4)=0.1; R_{f} (compound 15-6)=0.5.

Step 6: A mixture of compound 15-6 (500 mg, 0.877 mmol) and CeCl₃.7H₂O (160 mg, 0.430 mmol) in isopropanol (5 mL) was stirred at 75° C. under oxygen atmosphere for 3 h. After the completion of the reaction, the mixture was concentrated. The residue was purified by silica gel column (eluted with 5% MeOH in DCM) to afford the title compound 15-7 (420 mg, yield 81%) as a pale-yellow oil. TLC (DCM:MeOH/20:1): R_{f} (compound 15-6)=0.65; R_{f} (compound 15-7)=0.3.

Step 7: A solution of compound 15-7 (420 mg, 0.717 mmol) and TBAF (1 M THE solution, 4 mL, 4 mmol) in DMF (4 mL) was stirred at 100° C. under nitrogen atmosphere for 2 h. After the completion of the reaction, the mixture was cooled to room temperature and diluted by EtOAc (50 mL) and water (50 mL). The mixture was separated and the aqueous layer was re-extracted by EtOAc (50 mL). The organic layers were washed by 1 N HCl, saturated NaHCO₃ and brine, dried over anhydrous Na₂SO₄, filtered and concentrated. The residue was purified by silica gel column (eluted with 5% MeOH in DCM) to afford 15-8 (75 mg, yield 23%) as a white powder. ¹H NMR (400 MHz, Chloroform-d) δ 8.69 (s, 1H), 7.67 (d, J=6.2 Hz, 1H), 7.46 (s, 1H), 7.21 (d, J=8.5 Hz, 1H), 6.84-6.69 (m, 3H), 6.63 (d, J=9.3 Hz, 1H), 5.25 (s, 1H), 4.46-4.33 (m, 2H), 4.11 (d, J=8.5 Hz, 1H), 3.80 (d, J=9.9 Hz, 1H), 2.81-2.71 (m, 1H), 2.34-2.24 (m, 1H), 2.09 (d, J=9.2 Hz, 2H), 2.00 (s, 2H), 1.91 (s, 2H), 1.83 (s, 2H). LCMS: m/z calculated for C₂₄H₂₃F₂N₃O₄: 455.46; found: 456.17 [M+H]⁺. TLC (DCM:MeOH/20:1): R_{f} (compound 15-7)=0.3, R_{f} (15-8)=0.2.

Step 8: Similar to the chiral separation procedure for compound 1 and 1a, compound 15-8 was applied to a Chiralcel OD column to afford compound 15 and 15a.

### Example 16

### (S)-N-(3,5-difluorobenzyl)-3-hydroxy-2-oxo-1-(2′-oxospiro[cyclopropane-1,3′-indoline]-5′-yl )pyrrolidine-3-carboxamide (16)

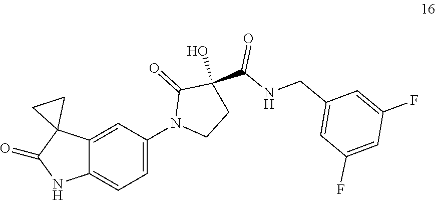 [SMILES: O=C1Nc2ccc(N3CC[C@](O)(C(=O)NCc4cc(F)cc(F)c4)C3=O)cc2C12CC2]

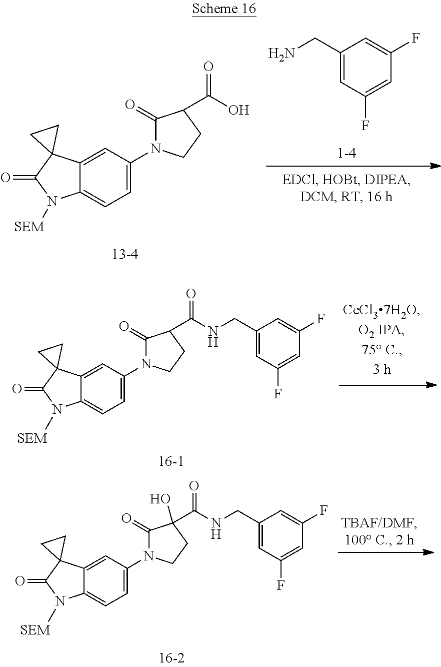 [SMILES: C[Si](C)(C)CCOCN1C(=O)C2(CC2)c2cc(N3CCC(C(=O)O)C3=O)ccc21 ; NCc1cc(F)cc(F)c1 ; C[Si](C)(C)CCOCN1C(=O)C2(CC2)c2cc(N3CCC(C(=O)NCc4cc(F)cc(F)c4)C3=O)ccc21 ; C[Si](C)(C)CCOCN1C(=O)C2(CC2)c2cc(N3CCC(O)(C(=O)NCc4cc(F)cc(F)c4)C3=O)ccc21]

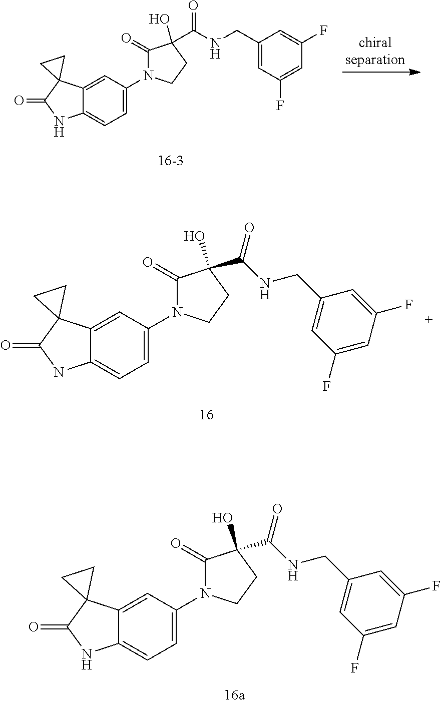 [SMILES: O=C(NCc1cc(F)cc(F)c1)C1(O)CCN(c2ccc3c(c2)C2(CC2)C(=O)N3)C1=O ; O=C1Nc2ccc(N3CC[C@](O)(C(=O)NCc4cc(F)cc(F)c4)C3=O)cc2C12CC2 ; O=C1Nc2ccc(N3CC[C@@](O)(C(=O)NCc4cc(F)cc(F)c4)C3=O)cc2C12CC2]

Step 1: A mixture of compound 13-4 (300 mg, 0.72 mmol), compound 1-4 (150 mg, 1.04 mmol), HOBt (97 mg, 0.72 mmol), DIPEA (218 mg, 1.69 mmol) and EDCI (250 mg, 1.31 mmol) in anhydrous DCM (3 mL) was stirred at room temperature for 16 h. After the completion of the reaction, the mixture was diluted by EtOAc (20 mL) and washed with water, 1 N HCl and brine. The organic layer was dried over anhydrous Na₂SO₄, filtered and concentrated. The residue was purified by silica gel column (eluted with 30% EtOAc in petroleum ether) to afford the title compound 10 (310 mg, yield 76%) as a foam. TLC (Petroleum ether:EtOAc/2:1): R_{f} (compound 1-4)=0.1; R_{f} (compound 16-1)=0.4.

Step 2: A mixture of compound 16-1 (310 mg, 0.572 mmol) and CeCl₃.7H₂O (106 mg, 0.285 mmol) in isopropanol (6 mL) was stirred at 75° C. under oxygen atmosphere for 3 h. After the completion of the reaction, the mixture was concentrated. The residue was purified by silica gel column (eluted with 5% MeOH in DCM) to afford the title compound 16-2 (193 mg, yield 60%) as a white solid. TLC (DCM:MeOH/20:1): R_{f} (compound 16-1)=0.5; R_{f} (compound 16-2)=0.4.

Step 3: A solution of compound 16-2 (190 mg, 0.341 mmol) and TBAF (1 M THE solution, 1.7 mL, 1.7 mmol) in DMF (2 mL) was stirred at 100° C. under nitrogen atmosphere for 2 h. After the completion of the reaction, the mixture was cooled to room temperature and diluted by EtOAc (20 mL) and water (20 mL). The mixture was separated, the organic layer was washed by 1 N HCl, saturated NaHCO₃ and brine, dried over anhydrous Na₂SO₄, filtered and concentrated. The residue was purified by silica gel column (eluted with 5% MeOH in DCM) to afford 16-3 (50 mg, yield 34%) as a white powder. ¹H NMR (400 MHz, Methanol-d₄) δ 7.35 (dd, J=8.4, 2.2 Hz, 1H), 7.22 (d, J=2.1 Hz, 1H), 6.97 (d, J=8.3 Hz, 1H), 6.94-6.88 (m, 2H), 6.78 (ddd, J=9.3, 8.0, 2.4 Hz, 1H), 4.47 (d, J=15.7 Hz, 1H), 4.36 (d, J=15.6 Hz, 1H), 3.94 (dt, J=9.7, 7.5 Hz, 1H), 3.86 (dt, J=12.6, 6.2 Hz, 1H), 2.69 (ddd, J=13.4, 7.7, 3.5 Hz, 1H), 2.21 (ddd, J=13.0, 8.8, 7.1 Hz, 1H), 1.61 (d, J=3.4 Hz, 4H). LCMS: m/z calculated for C₂₂H₁₉F₂N₃O₄: 427.41; found: 428.04 [M+H]⁺. TLC (DCM:MeOH/20:1): R_{f} (compound 16-2)=0.4, R_{f} (16-3)=0.3.

Step 4: Similar to the chiral separation procedure for compound 1 and 1a, compound 16-3 was applied to a Chiralcel OD column to afford compound 16 and 16a.

### Example 17

### (S)-N-(3-chloro-5-fluorobenzyl)-3-hydroxy-2-oxo-1-(2′-oxo-1′,2′-dihydrospiro[cyclopropane-1,3′-pyrrolo[2,3-b]pyridine]-5′-yl)pyrrolidine-3-carboxamide (17)

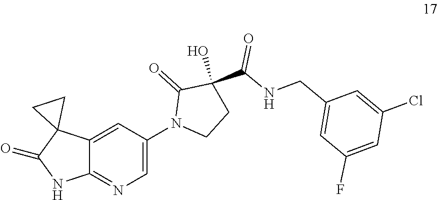 [SMILES: O=C1Nc2ncc(N3CC[C@](O)(C(=O)NCc4cc(F)cc(Cl)c4)C3=O)cc2C12CC2]

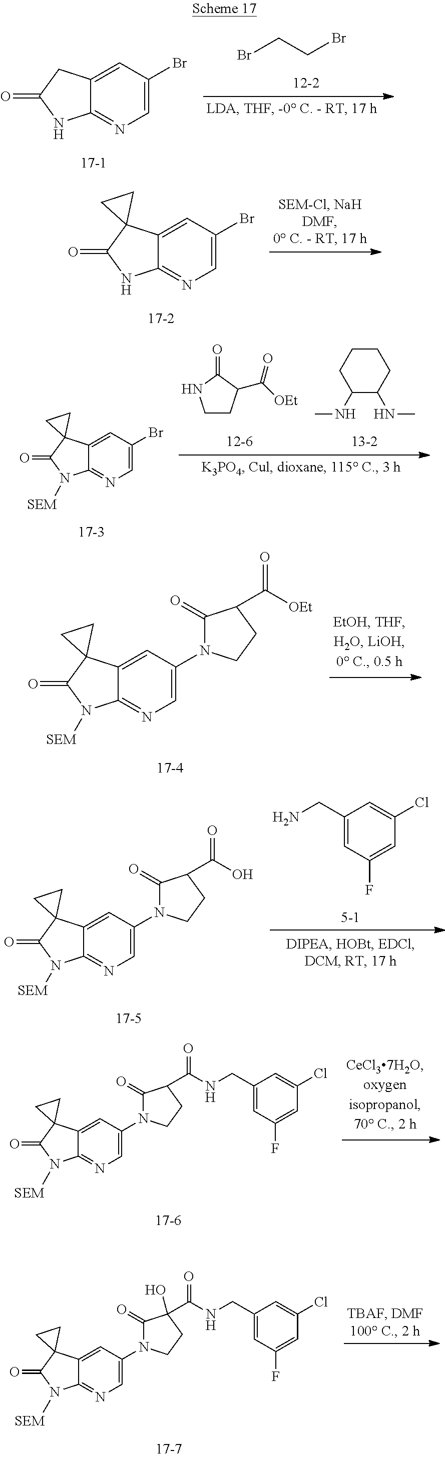

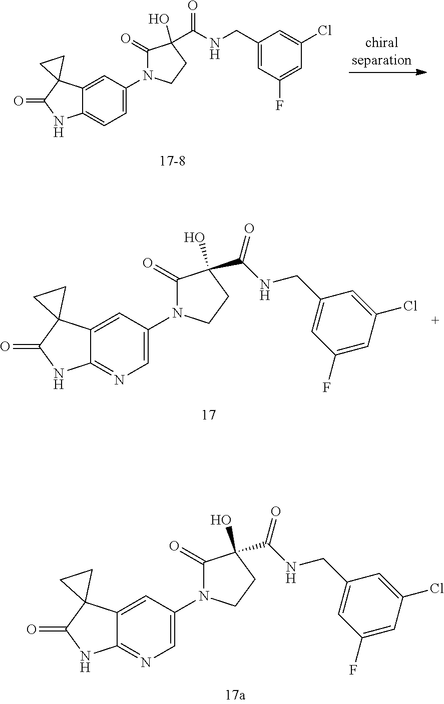 [SMILES: O=C(NCc1cc(F)cc(Cl)c1)C1(O)CCN(c2ccc3c(c2)C2(CC2)C(=O)N3)C1=O ; O=C1Nc2ncc(N3CC[C@](O)(C(=O)NCc4cc(F)cc(Cl)c4)C3=O)cc2C12CC2 ; O=C1Nc2ncc(N3CC[C@@](O)(C(=O)NCc4cc(F)cc(Cl)c4)C3=O)cc2C12CC2]

Step 1: To a solution of compound 17-1 (2.0 g, 9.39 mmol) in anhydrous THF (10 mL) at −5° C. was added dropwise LDA (2 M in THF solution, 18.6 mL, 37.56 mmol) under nitrogen atmosphere. After stirring at −5° C. for 30 min, compound 12-2 (5.29 g, 28.16 mmol) was added in one portion. Then the mixture was stirred at room temperature for 17 h. After the completion of the reaction, water (40 mL) was added slowly to quench the reaction and the mixture was extracted with EtOAc (30 mL×2). The organic layers were washed with brine, dried by anhydrous Na₂SO₄, filtered and concentrated. The crude product was purified by silica gel column (Petroleum ether:EtOAc=5:1) to afford the title compound 17-2 (480 mg, yield 21.4%) as a pink solid. TLC (Petroleum ether:EtOAc/2:1): R_{f} (compound 17-1): =0.3, R_{f} (compound 17-2): =0.4.

Step 2: To a mixture of compound 17-2 (480 mg, 2.01 mmol) in anhydrous DMF (4 mL) was added NaH (60% in mineral oil, 241 mg, 6.02 mmol) in several portions at 0° C. under nitrogen. After stirring at for 0° C. for 30 min, SEM-Cl (670 mg, 4.01 mmol) was added to the mixture. The reaction mixture was stirred at room temperature for 17 h. After the completion of the reaction, the mixture was partitioned between EtOAc (40 mL) and water (40 mL). The organic layer was washed with brine (40 mL), dried over anhydrous Na₂SO₄ and concentrated. The crude product was purified by silica gel column (eluted with 20% EtOAc in petroleum ether) to afford the title compound 17-3 (300 mg, yield 40%) as a colorless oil. TLC (Petroleum ether:EtOAc/5:1): R_{f} (compound 17-2): =0.1, R_{f} (compound 17-3): =0.5.

Step 3: While under nitrogen, a mixture of compound 17-3 (300 mg, 0.812 mmol), compound 12-6 (128 mg, 0.812 mmol), compound 13-2 (116 g, 0.821 mmol), CuI (155 mg, 0.812 mml) and K₃PO₄ (604 mg, 2.84 mmol) in dioxane (3 mL) was stirred at 115° C. for 3 h. After the completion of the reaction, the mixture was filtered over celite. The filtrate was diluted with water (20 mL) and extracted with EtOAc (20 mL×2). The combined organic layers were washed with water and brine, dried over anhydrous Na₂SO₄, filtered and concentrated. The residue was purified by silica gel column (eluted with 50% EtOAc in petroleum ether) to afford the title compound 17-4 (204 mg, yield 56.5%) as a colorless oil. TLC (Petroleum ether:EtOAc/2:1): R_{f} (compound 17-3): =0.9, R_{f} (compound 17-4): =0.1.

Step 4: To a solution of compound 17-4 (200 mg, 0.449 mmol) in EtOH (1.0 mL), THE (0.5 mL) and H₂O (1.0 mL) was added LiOH·H₂O (38 mg, 0.898 mmol) at 0° C. After the reaction was stirred at room temperature for 0.5 h, the mixture was concentrated to get rid of organic solvents. The left aqueous solution was adjusted to pH=2 and extracted with EtOAc (20 mL×2). The organic layers were washed with brine, dried over anhydrous Na₂SO₄, filtered and concentrated to afford the title compound 17-5 (143 mg, yield 75%) as a colorless oil, which was used directly at the next step. TLC (DCM:MeOH/20:1): R_{f} (compound 17-4): =0.5, R_{f} (compound 17-5): =0.25.

Step 5: Under nitrogen atmosphere, a mixture of compound 17-5 (140 mg, 0.335 mmol), compound 5-1 (64.0 mg, 0.402 mmol), HOBt (45 mg, 0.335 mmol), DIPEA (86 mg, 0.67 mmol) and EDCI (96 g, 0.503 mmol) in anhydrous DCM (1 mL) was stirred at room temperature for 17 h. After the completion of the reaction, the mixture was diluted by EtOAc (20 mL) and washed with water, 1 N HCl and brine. The organic layer was dried over anhydrous Na₂SO₄, filtered and concentrated. The residue was purified by silica gel column (eluted with 5% MeOH in DCM) to afford the title compound 17-6 (107 mg, yield 57%) as a colorless oil. TLC (DCM:MeOH/10:1): R_{f} (compound 17-5): =0.25, R_{f} (compound 17-6): =0.4.

Step 6: A mixture of compound 17-6 (105 mg, 0.188 mmol) and CeCl₃.7H₂O (70 mg, 0.188 mmol) in isopropanol (2 mL) was stirred at 70° C. under oxygen atmosphere for 2 h. After the completion of the reaction, the mixture was concentrated. The residue was purified by silica gel column (eluted with 5% MeOH in DCM) to afford the title compound 17-7 (80 mg, yield 78%) as a colorless oil. TLC (DCM:MeOH/20:1): R_{f} (compound 17-6): =0.3, R_{f} (compound 17-7): =0.25.

Step 7: A solution of compound 17-7 (80 mg, 0.14 mmol) and TBAF (1 M THE solution, 2.78 mL, 2.78 mmol) in DMF (1 mL) was stirred at 100° C. under nitrogen atmosphere for 2 h. The mixture was cooled to room temperature and diluted by EtOAc (10 mL) and water (10 mL). The mixture was separated, the organic layer was washed with 1 N HCl, saturated NaHCO₃ solution and brine, dried over anhydrous Na₂SO₄, filtered and concentrated. The residue was purified by pre-HPLC (C18 column, eluted with acetonitrile/H₂O, HCl condition) to afford 17-8 (1.7 g, yield 3%). LCMS: m/z calculated for C₂₁H₁₈ClFN₄O₄: 444.1; found: 445.12. [M+H]⁺.

Step 8: Similar to the chiral separation procedure for compound 1 and 1a, compound 17-8 was applied to a Chiralcel OD column to afford compound 17 and 17a.

### Example 18

### (S)-N-(3,5-difluorobenzyl)-1-(2,2-dioxido-1H-spiro[benzo[c]isothiazole-3,1′-cyclobutan]-5-y 1)-3-hydroxy-2-oxopyrrolidine-3-carboxamide (18)

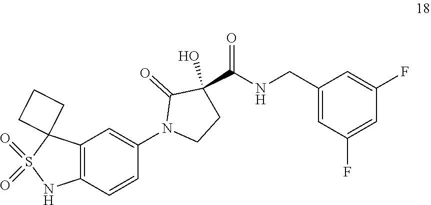 [SMILES: O=C(NCc1cc(F)cc(F)c1)[C@@]1(O)CCN(c2ccc3c(c2)C2(CCC2)S(=O)(=O)N3)C1=O]

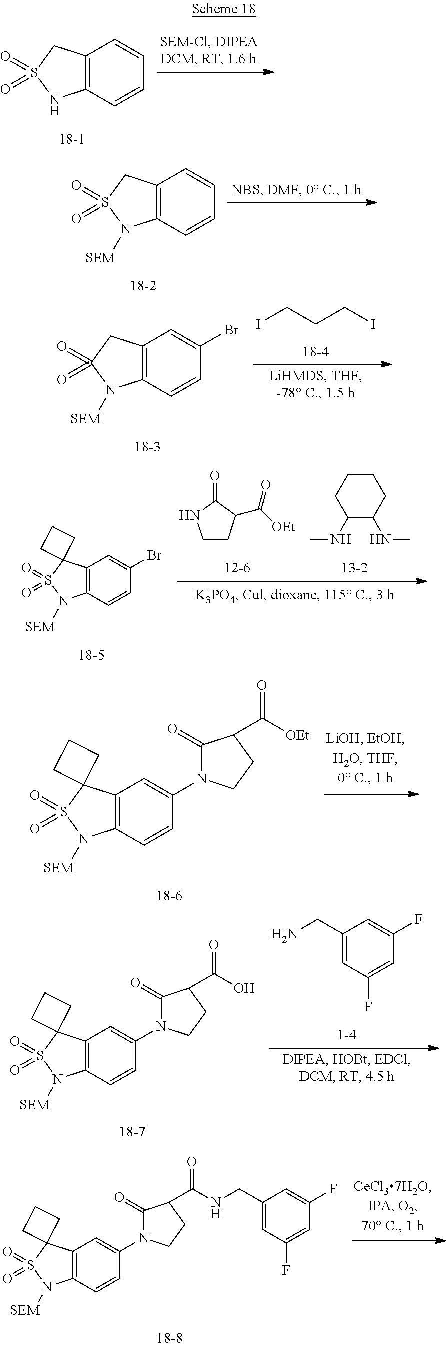

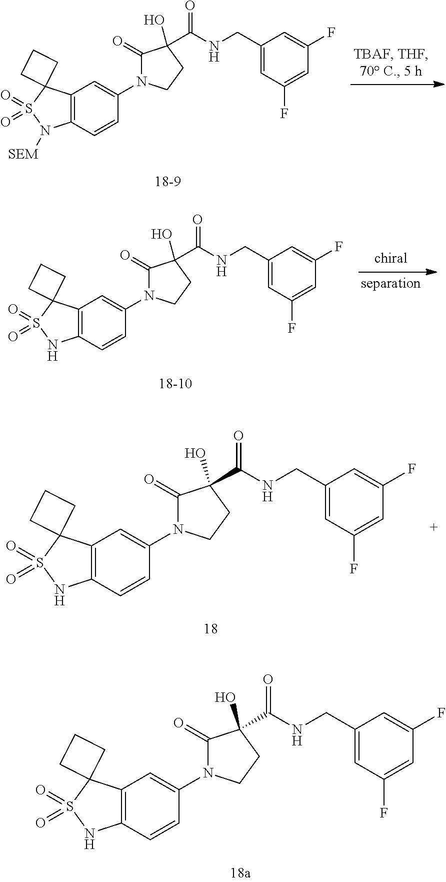 [SMILES: C[Si](C)(C)CCOCN1c2ccc(N3CCC(O)(C(=O)NCc4cc(F)cc(F)c4)C3=O)cc2C2(CCC2)S1(=O)=O ; O=C(NCc1cc(F)cc(F)c1)C1(O)CCN(c2ccc3c(c2)C2(CCC2)S(=O)(=O)N3)C1=O ; O=C(NCc1cc(F)cc(F)c1)[C@@]1(O)CCN(c2ccc3c(c2)C2(CCC2)S(=O)(=O)N3)C1=O ; O=C(NCc1cc(F)cc(F)c1)[C@]1(O)CCN(c2ccc3c(c2)C2(CCC2)S(=O)(=O)N3)C1=O]

Step 1: To a solution of compound 18-1 (2.0 g, 11.8 mmol) and DIPEA (3.0 g, 23.2 mmol) in anhydrous DCM (20 mL) at room temperature was added SEM-Cl (2.9 g, 14.4 mmol). After stirring for 1.5 h, the reaction mixture was diluted with DCM (30 mL) and washed with brine. The organic layer was dried over anhydrous Na₂SO₄ and concentrated. The crude product was purified by column (eluted with 15% EtOAc in petroleum ether) to afford compound 18-2 (2.9 g, yield 83%) as a white solid. TLC (Petroleum ether:EtOAc/5:1): R_{f} (compound 18-1)=0.1; R_{f}(compound 18-2)=0.37.

Step 2: To a solution of compound 18-2 (1.0 g, 3.34 mmol) in anhydrous DMF (10 mL) at 0° C. was added NBS (713 mg, 4.00 mmol). After stirring at room temperature for 1 h, the reaction mixture was diluted with water (50 mL) and extracted with EtOAc (50 mL×2). The organic layers were washed with brine, dried over anhydrous Na₂SO₄ and concentrated. The crude product was purified by column (eluted with 15% EtOAc in petroleum ether) to afford compound 18-3 (1.1 g, yield 87%) as a yellow solid. TLC (Petroleum ether:EtOAc/5:1): R_{f}(compound 18-2)=0.2; R_{f} (compound 18-3)=0.3.

Step 3: Under nitrogen atmosphere, to a mixture of compound 18-3 (770 mg, 2.04 mmol) and compound 18-4 (1.21 g, 4.07 mmol) in anhydrous THE (5 mL) was added dropwise LiHMDS (1 M, 6.11 mL, 6.11 mmol) at −78° C. After the reaction was stirred at −78° C. for 1 h, the mixture was quenched by addition of cold water and extracted with EtOAc (20 mL×2). The organic layers were washed with brine, dried over anhydrous Na₂SO₄ and concentrated. The crude product was purified by column (eluted with 5% EtOAc in petroleum ether) to afford compound 18-5 (477 mg, yield 56%) as a yellow oil. TLC (Petroleum ether:EtOAc/20:1): R_{f}(compound 18-3)=0.2; R_{f} (compound 18-5)=0.3.

Step 4: Under nitrogen atmosphere, a mixture of compound 18-5 (474 mg, 1.13 mmol), compound 12-6 (230 mg, 1.46 mmol), compound 13-2 (160 mg, 1.12 mmol), CuI (215 mg, 1.27 mmol) and K₃PO₄ (720 mg, 3.39 mmol) in anhydrous dioxane (4 mL) was stirred at 115° C. for 3.5 h. After the reaction was completed, the mixture was filtered. The filtrate was diluted with water (30 mL) and extracted with EtOAc (30 mL×2). The organic layers were washed with brine, dried over anhydrous Na₂SO₄ and concentrated. The crude product was purified by column (eluted with 30% EtOAc in petroleum ether) to afford compound 18-6 (268 mg, yield 48%) as a yellow oil. TLC (Petroleum ether:EtOAc/2:1): R_{f} (compound 18-5)=0.7; R_{f} (compound 18-6)=0.3.

Step 5: To a solution of compound 18-6 (268 mg, 0.54 mmol) in EtOH (1.2 mL) and THE (0.6 mL) was added a solution of LiOH·H₂O (45.5 mg, 1.08 mmol) in water (1.2 mL) at 0° C. After stirring at room temperature for 1 h, the mixture was concentrated under reduced pressure to remove organic solvents. The aqueous solution was acidified with 1N HCl to pH=3 and then extracted with EtOAc (20 mL×2). The organic layers were washed with brine, dried over anhydrous Na₂SO₄ and concentrated to afford compound 18-7 (191 mg, yield 75%) as a yellow oil. TLC (DCM:MeOH/20:1): R_{f} (compound 18-6)=0.4; R_{f} (compound 18-7)=0.1.

Step 6: A mixture of compound 18-7 (241 mg, 0.52 mmol), DIPEA (0.27 mL, 1.56 mmol), compound 1-4 (110.7 mg, 0.78 mmol), HOBt (70.1 mg, 0.52 mmol) and EDCI (295.8 mg, 1.56 mmol) in DCM (5 mL) was stirred at room temperature for 16 h. After the reaction was completed, the mixture was diluted with EtOAc (30 mL) and washed with 1 N HCl (30 mL). The organic layer was washed with brine, dried over anhydrous Na₂SO₄ and concentrated. The crude product was purified by column (eluted with 50% EtOAc in petroleum ether) to afford compound 18-8 (188 mg, yield 61%) as a yellow oil. TLC (DCM:MeOH/10:1): R_{f} (compound 18-7)=0.2; R_{f} (compound 18-8)=0.4.

Step 7: A mixture of compound 18-8 (188 mg, 0.32 mmol) and CeCl₃.7H₂O (59.2 mg, 0.16 mmol) in isopropanol (3 mL) was stirred under oxygen atmosphere at 70° C. for 1 h. After the reaction was completed, the mixture was cooled to room temperature and concentrated. The crude product was purified by column (eluted with 70% EtOAc in petroleum ether) to afford compound 18-9 (89 mg, yield 46%) as a yellow oil. TLC (DCM:MeOH/20:1): R_{f} (compound 18-8)=0.5; R_{f} (compound 18-9)=0.4.

Step 8: A mixture of compound 18-9 (89 mg, 0.15 mmol) and TBAF (1 Min THF, 1.9 mL, 1.9 mmol) was stirred at 70° C. under nitrogen atmosphere for 5 h. The reaction mixture was cooled to room temperature and diluted with EtOAc (10 mL). The mixture was washed with water and brine. The organic layer was dried over anhydrous Na₂SO₄ and concentrated. The residue was re-dissolved in acetonitrile and water, and purified by HPLC (C18 column, eluted with acetonitrile/H₂O, TFA condition). The desired component was lyophilized to give compound 18-10 (5.7 mg, yield 8%) as a white solid. ¹H NMR (500 MHz, DMSO-d₆) δ 10.42 (s, 1H), 8.72 (t, J=6.4 Hz, 1H), 7.88 (d, J=2.4 Hz, 1H), 7.58 (dd, J=8.6, 2.3 Hz, 1H), 7.07 (tt, J=9.4, 2.4 Hz, 1H), 7.03-6.94 (m, 2H), 6.84 (d, J=8.6 Hz, 1H), 6.75 (s, 1H), 4.40 (dd, J=15.8, 6.7 Hz, 1H), 4.26 (dd, J=15.8, 6.0 Hz, 1H), 3.95-3.84 (m, 2H), 2.91-2.81 (m, 2H), 2.64-2.58 (m, 1H), 2.47-2.43 (m, 1H), 2.21-1.94 (m, 4H). LCMS: m/z calculated for C₂₂H₂₁F₂N₃O₅S: 477.48; found: 478.26. [M+H]⁺.

Step 9: Similar to the chiral separation procedure for compound 1 and 1a, compound 18-10 was applied to a Chiralcel OD column to afford compound 18 and 18a.

### Example 19

### (S)-N-(3-chloro-5-fluorobenzyl)-1-(2,2-dioxido-1H-spiro[benzo[c]isothiazole-3,1′-cyclobuta n]-5-yl)-3-hydroxy-2-oxopyrrolidine-3-carboxamide (19)

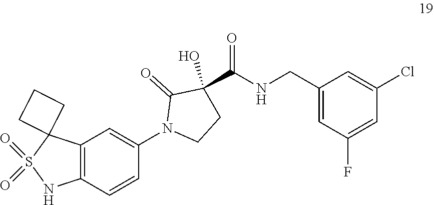 [SMILES: O=C(NCc1cc(F)cc(Cl)c1)[C@@]1(O)CCN(c2ccc3c(c2)C2(CCC2)S(=O)(=O)N3)C1=O]

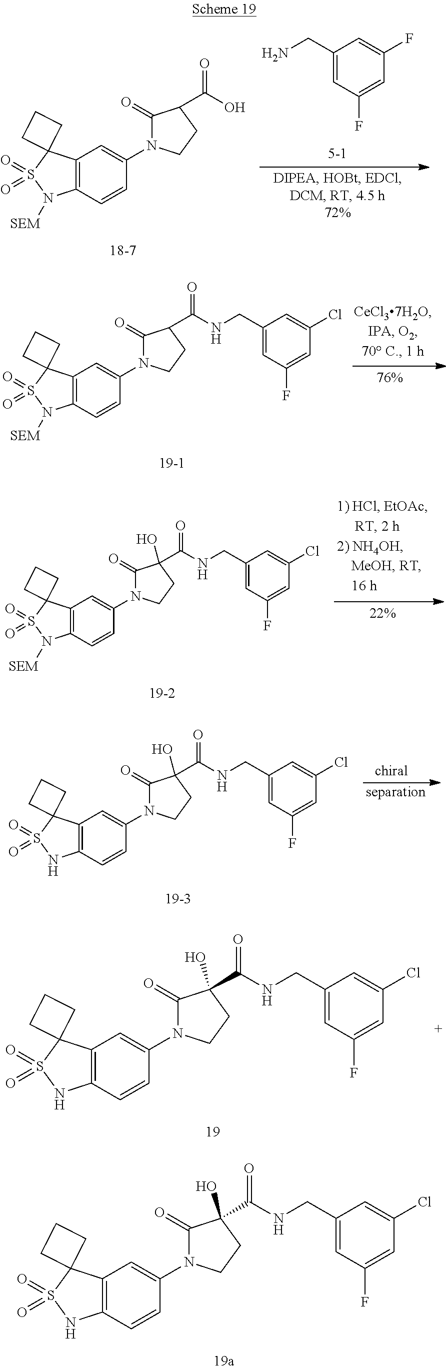

Step 1: A mixture of compound 18-7 (138 mg, 0.30 mmol), DIPEA (115 mg, 0.89 mmol), compound 5-1 (70.8 mg, 0.44 mmol), HOBt (39.9 mg, 0.30 mmol) and EDCI (169.5 mg, 0.89 mmol) and in DCM (5 mL). was stirred at room temperature for 16 h. After the reaction was completed, the mixture was diluted with EtOAc (30 mL) and washed with 1 N HCl (30 mL). The organic layer was washed with brine, dried over anhydrous Na₂SO₄ and concentrated. The crude product was purified by column (eluted with 50% EtOAc in petroleum ether) to afford compound 19-1 (130 mg, yield 72%) as a yellow oil. TLC (DCM:MeOH/10:1): R_{f} (compound 18-7)=0.2; R_{f} (compound 19-1)=0.4.

Step 2: A mixture of compound 19-1 (130 mg, 0.21 mmol) and CeCl₃ 7H₂O (39.9 mg, 0.11 mmol) in isopropanol (3 mL) was stirred under oxygen atmosphere at 70° C. for 1 h. After the reaction was completed, the mixture was cooled to room temperature and concentrated. The crude product was purified by column (eluted with 70% EtOAc in petroleum ether) to afford compound 19-2 (102 mg, yield 76%) as a yellow oil. TLC (DCM:MeOH/20:1): R_{f} (compound 19-1)=0.5; R_{f} (compound 19-2)=0.4.

Step 3: To a solution of compound 19-2 (45 mg, 0.072 mmol) in EtOAc (0.5 mL) was added a solution of HCl in EtOAc (4 N, 0.5 mL). After stirring at room temperature for 2 h, the mixture was concentrated. The residue was re-dissolved in MeOH (0.5 mL) and treated with NH₃ H₂O (0.2 mL). The mixture was then stirred at room temperature for 16 h. After the completion of the reaction, the mixture was concentrated. The residue was purified by pre-HPLC (C18 column, eluted with acetonitrile/H₂O, TFA condition). The desired component was lyophilized to give 19-3 (7.8 mg, yield 22%) as a white powder. ¹H NMR (600 MHz, DMSO-d₆) δ 10.42 (s, 1H), 8.73 (t, J=6.4 Hz, 1H), 7.90 (d, J=2.3 Hz, 1H), 7.58 (dd, J=8.6, 2.3 Hz, 1H), 7.28 (dt, J=8.7, 2.2 Hz, 1H), 7.22 (d, J=1.7 Hz, 1H), 7.11 (dt, J=9.6, 1.8 Hz, 1H), 6.84 (d, J=8.6 Hz, 1H), 6.76 (s, 1H), 4.39 (dd, J=15.7, 6.8 Hz, 1H), 4.26 (dd, J=15.7, 6.0 Hz, 1H), 3.92-3.86 (m, 2H), 2.89-2.83 (m, 2H), 2.62-2.58 (m, 1H), 2.46-2.43 (m, 1H), 2.22-1.94 (m, 4H). LCMS: m/z calculated for C₂₂H₂₁ClFN₃05S: 493.93; found: 494.04. [M+H]⁺.

Step 4: Similar to the chiral separation procedure for compound 1 and 1a, compound 19-3 was applied to a Chiralcel OD column to afford compound 19 and 19a.

### Example 20

### (S)-N-(3-chloro-5-fluorobenzyl)-1-(2,2-dioxido-1H-spiro[benzo[c]isothiazole-3,1′-cycloprop an]-5-yl)-3-hydroxy-2-oxopyrrolidine-3-carboxamide (20)

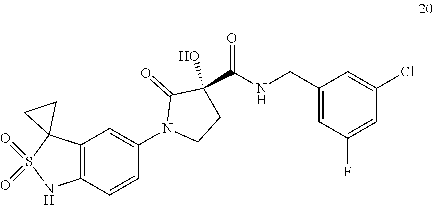 [SMILES: O=C(NCc1cc(F)cc(Cl)c1)[C@@]1(O)CCN(c2ccc3c(c2)C2(CC2)S(=O)(=O)N3)C1=O]

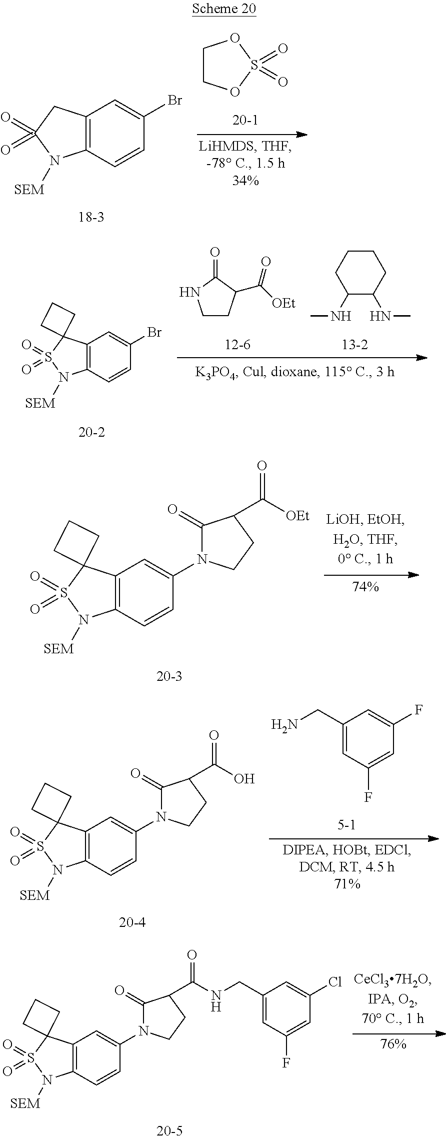

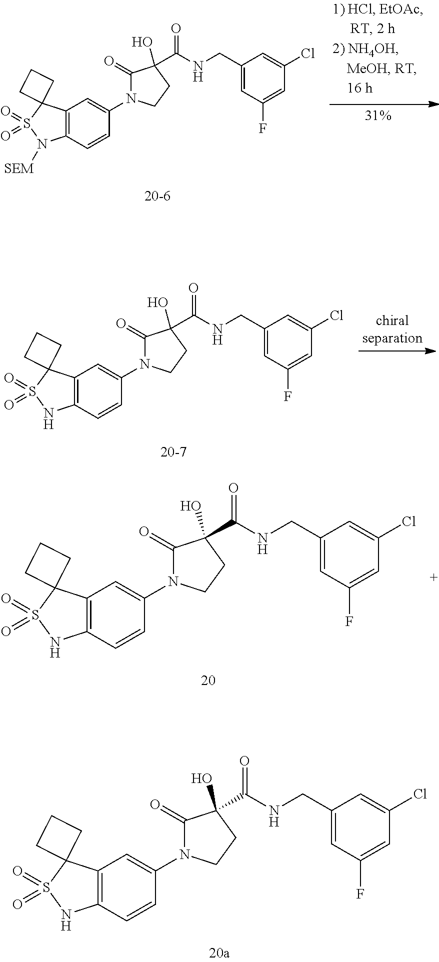 [SMILES: C[Si](C)(C)CCOCN1c2ccc(N3CCC(O)(C(=O)NCc4cc(F)cc(Cl)c4)C3=O)cc2C2(CCC2)S1(=O)=O ; O=C(NCc1cc(F)cc(Cl)c1)C1(O)CCN(c2ccc3c(c2)C2(CCC2)S(=O)(=O)N3)C1=O ; O=C(NCc1cc(F)cc(Cl)c1)[C@@]1(O)CCN(c2ccc3c(c2)C2(CCC2)S(=O)(=O)N3)C1=O ; O=C(NCc1cc(F)cc(Cl)c1)[C@]1(O)CCN(c2ccc3c(c2)C2(CCC2)S(=O)(=O)N3)C1=O]

Step 1: Under nitrogen atmosphere, to a mixture of compound 18-3 (550 mg, 1.45 mmol) and compound 20-1 (327.9 mg, 2.91 mmol) in anhydrous THF (5 mL) was added slowly LiHMDS (1 M, 4.5 mL, 4.5 mmol) at −78° C. Then the mixture was stirred at −78° C. for 1 h. After the completion of the reaction, the mixture was diluted with EtOAc, quenched by addition of ice water, and separated. The organic layer was washed with brine, dried over anhydrous Na₂SO₄ and concentrated. The crude product was purified by silica gel column (eluted with 2% EtOAc in petroleum ether) to afford compound 20-2 (203 mg, yield 34%) as a yellow oil. TLC (Petroleum ether:EtOAc/20:1): R_{f} (compound 18-3)=0.2; R_{f} (compound 20-2)=0.3.

Step 2: Under nitrogen atmosphere, a mixture of compound 20-2 (287 mg, 0.71 mmol), compound 12-6 (145 mg, 0.92 mmol), compound 13-2 (100 mg, 0.71 mmol), CuI (135 mg, 0.71 mmol) and K₃PO₄ (452 mg, 2.13 mmol) in anhydrous dioxane (4 mL) was stirred at 115° C. for 3.5 h. After the reaction was completed, the mixture was filtered. The filtrate was diluted with water (30 mL) and extracted with EtOAc (30 mL×2). The organic layers were washed with brine, dried over anhydrous Na₂SO₄ and concentrated. The crude product was purified by column (eluted with 30% EtOAc in petroleum ether) to afford compound 20-3 (209 mg, yield 61%) as a yellow oil. TLC (Petroleum:EtOAc/2:1): R_{f} (compound 20-2)=0.7; R_{f} (compound 20-3)=0.3.

Step 3: A mixture of compound 20-3 (204 mg, 0.42 mmol) in EtOH (1.0 mL) and THE (0.5 mL) and was added a solution of LiOH·H₂O (35.6 mg, 0.85 mmol) in H₂O (1.0 mL). The reaction was stirred at 0° C. for 1 h. The mixture was concentrated, acidified with 1N HCl to pH=3 and then extracted with EtOAc (20 mL×2). The organic layers were washed with brine, dried over anhydrous Na₂SO₄ and concentrated to afford the title compound 20-4 (143 mg, yield 74%) as a yellow oil. TLC (DCM:MeOH/20:1): R_{f} (compound 20-3)=0.4; R_{f} (compound 20-4)=0.1.

Step 4: A mixture of compound 20-4 (128 mg, 0.28 mmol), DIPEA (0.15 mL, 0.85 mmol), and compound 5-1 (54.1 mg, 0.31 mmol), HOBt (38.2 mg, 0.28 mmol) and EDCI (162.1 mg, 0.85 mmol) in anhydrous DCM (4 mL) was stirred at room temperature for 2 h. After the completion of the reaction. The mixture was diluted with EtOAc and washed with 1 N HCl and brine. The organic layer was dried over anhydrous Na₂SO₄ and concentrated. The crude product was purified by pre-TLC (SiO₂, DCM:MeOH/20:1) to afford compound 20-5 (120 mg, yield 71%) as a yellow oil. TLC (DCM/MeOH/10:1): R_{f} (compound 20-4)=0.1; R_{f} (compound 20-5)=0.4.

Step 5: A mixture of compound 20-5 (115 mg, 0.19 mmol) and CeCl₃ 7H₂O (36 mg, 0.10 mmol) in isopropanol (2 mL) was stirred at 70° C. under oxygen for 1 h. After the completion of the reaction, the mixture was concentrated. The crude product was purified by pre-TLC (SiO₂, DCM:MeOH/20:1) to afford compound 20-6 (70 mg, yield 59%) as a yellow oil. TLC (DCM:MeOH/20:1): R_{f} (compound 20-5)=0.5; R_{f} (compound 20-6)=0.4.

Step 6: To a solution of compound 20-6 (65 mg, 0.11 mmol) in EtOAc (0.5 mL) was added a solution of HCl in EtOAc (4 N, 0.5 mL). After stirring at room temperature for 2 h, the mixture was concentrated. The residue was re-dissolved in MeOH (1 mL) and treated with NH₃·H₂O (1 mL). The mixture was then stirred at room temperature for 16 h. After the completion of the reaction, the mixture was concentrated. The residue was purified by pre-HPLC (C18 column, eluted with acetonitrile/H₂O, TFA condition). The desired component was lyophilized to give 20-7 (16 mg, yield 31%) as a white powder. ¹H NMR (600 MHz, DMSO-d₆) δ 10.69 (s, 1H), 8.71 (t, J=6.4 Hz, 1H), 7.63 (dd, J=8.6, 2.3 Hz, 1H), 7.27 (dt, J=8.8, 2.3 Hz, 1H), 7.23-7.15 (m, 2H), 7.10 (d, J=9.2 Hz, 1H), 6.89 (d, J=8.6 Hz, 1H), 6.73 (s, 1H), 4.37 (dd, J=15.7, 6.7 Hz, 1H), 4.25 (dd, J=15.7, 6.0 Hz, 1H), 3.85-3.77 (m, 2H), 2.57 (ddd, J=12.2, 7.0, 4.7 Hz, 1H), 2.10 (dt, J=13.0, 7.6 Hz, 1H), 1.77 (q, J=5.2 Hz, 2H), 1.63 (d, J=2.9 Hz, 2H). LCMS: m/z calculated for C₂₁H₁₉C1FN₃05S: 479.91; found: 480.69. [M+H]⁺.

Step 7: Similar to the chiral separation procedure for compound 1 and 1a, compound 20-7 was applied to a Chiralcel OD column to afford compound 20 and 20a.

### Example 21

### N-(3-chloro-5-fluorobenzyl)-3-hydroxy-2-oxo-1-(5H-pyrrolo[2,3-b]pyrazin-2-yl)pyrrolidine-3-carboxamide (21)

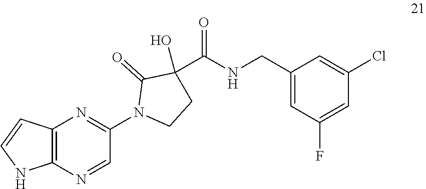 [SMILES: O=C(NCc1cc(F)cc(Cl)c1)C1(O)CCN(c2cnc3[nH]ccc3n2)C1=O]

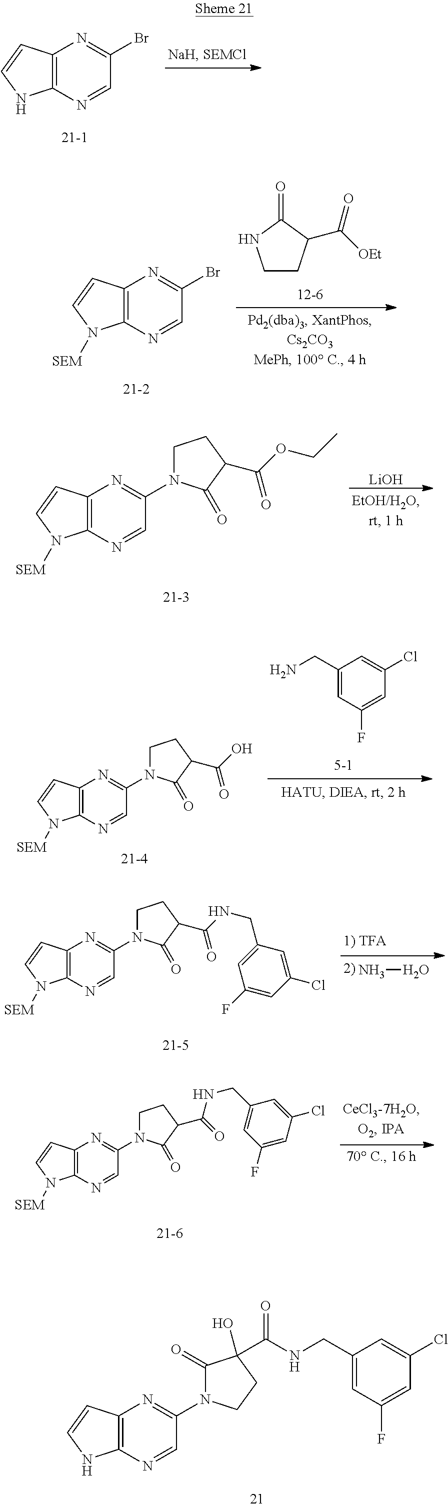

Step 1: To a solution of compound 21-1 (1 g, 5.05 mmol) in DMF (15 mL) was added 60% NaH (282.8 mg, 7.07 mmol) at 0° C. The reaction mixture stirred for 0.5 h at 0° C. Then the mixture was added SEMCl (1.1 g, 6.56 mmol) at 0° C., warmed to room temperature and stirred for 2 h. The reaction was quenched with water. The mixture was extracted with ethyl acetate (3*30 mL). The combined organics were washed with brine (100 mL*4), dried over sodium sulfate, filtered, and concentrated by rotary evaporation. The resulting residue was purified by column chromatography (15% EtOAc in Petroleum ether) to give the compound 21-2 (1.2 g) as a yellow solid.

Step 2: To a solution of 21-2 (700 mg, 2.13 mmol) in toluene (12 mL) was added ethyl 2-oxopyrrolidine-3-carboxylate 12-6 (368.57 mg, 2.35 mmol), Pd₂(dba)₃ (97 mg, 0.11 mmol), Xantphos (74 mg, 0.14 mmol) and Cs₂CO₃(2.1 g, 6.48 mmol). The reaction mixture was stirred for 4 h at 100° C. The reaction mixture was quenched with water (15 mL) and extracted by ethyl acetate (3*20 mL). The combined organics were washed with brine (50 mL*4), dried over sodium sulfate, filtered, and concentrated by rotary evaporation. The resulting residue was purified by column chromatography (20% EtOAc in Petroleum ether) to give the compound 21-3 (450 mg) as a white solid.

Step 3: To a solution of Compound 21-3 (450 mg, 1.11 mmol) was dissolved in a EtOH/H₂O (8 ml/3 ml), added LiGH (79.9 mg, 3.33 mmol) under nitrogen atmosphere. The reaction mixture was stirred for 1 h at room temperature. The combined organics were concentrated by rotary evaporation to give the compound 21-4 (400 mg) as a yellow solid.

Step 4: To a solution of Compound 21-4 (400 mg, 1.06 mmol) in DMF (7 mL) was added compound 5-1 (187 mg, 1.17 mmol), HATU (604.2 mg, 1.59 mmol) and DIEA (439 mg, 3.18 mmol). The reaction mixture was stirred at room temperature for 2 h. The progress of the reaction mixture was monitored by TLC. The reaction was quenched with water (15 mL). The mixture was extracted with ethyl acetate (3*20 mL). The combined organics were washed with brine (60 mL*4), dried over sodium sulfate, filtered, and concentrated by rotary evaporation. The resulting residue was purified by column chromatography to give the compound 21-5 (400 mg) was a white solid.

Step 5: To a solution of Compound 21-5 (200 mg, 0.39 mmol) was dissolved in TFA/DCM (2 mL/4 ml) under N₂. The reaction mixture was stirred at room temperature for 4 h. The mixture was concentrated by rotary evaporation. Then the reaction was added NH₃*H₂O/DCM (5 ml/5 ml). The reaction mixture was stirred at room temperature for 12 h. The combined organics were concentrated by rotary evaporation to give the compound 21-6 (100 mg) was a white solid.

Step 6: To a solution of Compound 21-6 (100 mg, 0.26 mmol) in IPA (5 mL) was added CeCl₃*7H₂O (67.5 mg, 0.18 mmol) under O₂. The reaction mixture was stirred at 70° C. for 12 h. The mixture was filtered through a pad of Celite, washed by EtOAc (20 mL). The filtration was concentrated, and the residue was purified by prep-HPLC to give 21 (26.9 mg) as a white solid. ¹H NMR (400 MHz, CD₃OD) δ8.77 (s, 1H), 8.06 (d, J=2.8 Hz, 1H), 7.93 (s, 1H), 6.93 (d, J=6.5 Hz, 2H), 6.86 (d, J=2.9 Hz, 1H), 6.79 (t, J=9.1 Hz, 1H), 4.52 (d, J=15.7 Hz, 1H), 4.36 (d, J=15.7 Hz, 1H), 4.14 (t, J=6.7 Hz, 2H), 2.81 (dd, J=13.1, 7.1 Hz, 1H), 2.42-2.31 (m, 1H).

### Example 22

### N-(3,5-difluorobenzyl)-3-hydroxy-2-oxo-1-(1H-pyrrolo[2,3-c]pyridin-5-yl)pyrrolidine-3-car boxamide (22)

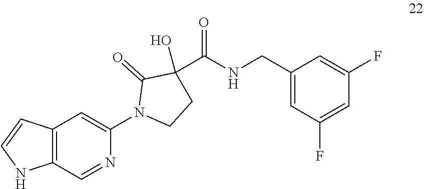 [SMILES: O=C(NCc1cc(F)cc(F)c1)C1(O)CCN(c2cc3cc[nH]c3cn2)C1=O]

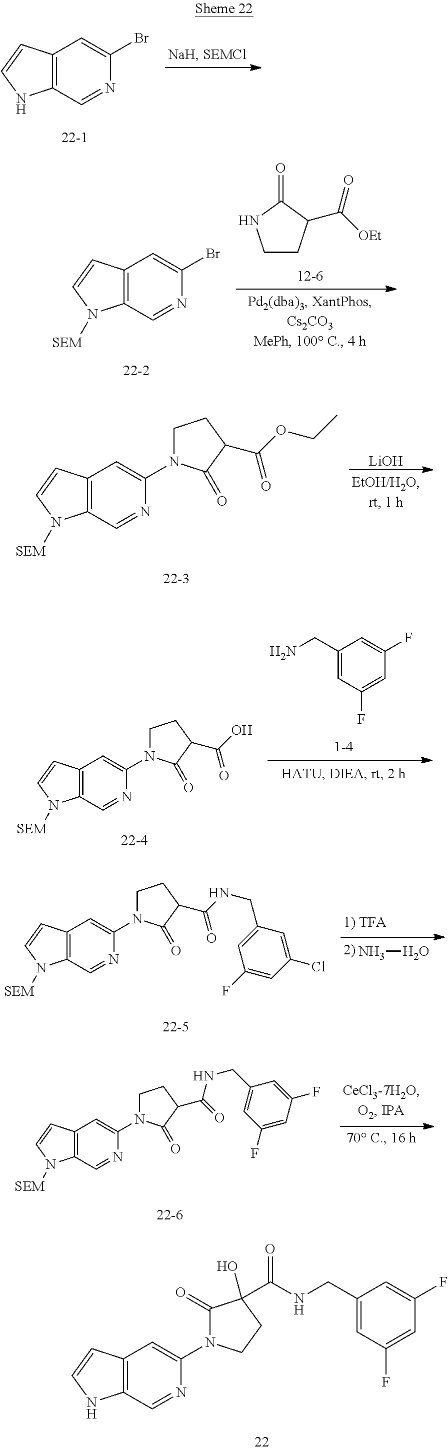

Step 1: To a solution of 5-bromo-1H-pyrrolo[2,3-c]pyridine 22-1 (1 g, 5.08 mmol) in DMF (15 mL) was added 60% NaH (0.325 g, 8.12 mmol) at 0° C. The reaction mixture stirred for 0.5h at 0° C. And the mixture was added SEMCl (1.1 g, 6.5 mmol) at 0° C. Then the reaction was warmed to room temperature and stirred for 2h. The reaction was quenched with water. The mixture was extracted with ethyl acetate (3*200 mL). The combined organic layers were washed with water and brine, dried over sodium sulfate, filtered, and concentrated by rotary evaporation. The resulting residue was purified by column chromatography to give the compound 22-2 (1.7 g) as a yellow oil that partially solidified under vacuum.

Step 2: To a solution of 22-2 (600 mg) in toluene (10 mL) was added ethyl 2-oxopyrrolidine-3-carboxylate 12-6 (511 mg), Pd₂(dba)₃ (90 mg) Xantphos (176 mg) and Cs₂CO₃(1.55 g). The reaction mixture was stirred for 36 h at 100° C. The reaction mixture was quenched with water and extracted by ethyl acetate (3*100 mL). The combined organic layers were washed with water and brine, dried over sodium sulfate, filtered, and concentrated by rotary evaporation. The resulting residue was purified by column chromatography to give the compound 22-3 (310 mg) as a white solid.

Step 3: To a solution of Compound 22-3 (270 mg) was dissolved in a mixture of EtOH/H₂O (4 ml/2 ml), added LiGH (56 mg) under nitrogen atmosphere. The reaction mixture was stirred for 1h at room temperature. The combined organics were concentrated by rotary evaporation to give the compound 22-4 (250 mg) as a yellow solid.

Step 4: To a solution of Compound 22-4 (250 mg) was dissolved in DMF (4 mL) was added compound 1-4 (123 mg), HATU (380 mg) and DIEA (257 mg). The reaction mixture was stirred at room temperature for 3h. The progress of the reaction mixture was monitored by TLC. The reaction was quenched with water. The mixture was extracted with ethyl acetate (3*200 mL). The combined organics were washed with water and brine, dried over sodium sulfate, filtered, and concentrated by rotary evaporation. The resulting residue was purified by column chromatography to give the compound 22-5 (390 mg) as a white solid.

Step 5: To a solution of Compound 22-5 (390 mg) was dissolved in TFA/DCM (10 ml/5 ml) under N₂. The reaction mixture was stirred at room temperature for 1h. The mixture was concentrated by rotary evaporation. Then the reaction was added NH₃*H₂O/DCM (5 ml/5 ml). The reaction mixture was stirred at room temperature for 12h. The combined organics were concentrated by rotary evaporation to give the compound 22-6 (215 mg) as a yellow solid.

Step 6: To a solution of Compound 22-6 (215 mg) in IPA (5 mL) was added CeCl₃*7H₂O (108 mg) under O₂. The reaction mixture was stirred at 70° C. for 12h. The reaction residue was purified by pre-HPLC to give 22 (80 mg) as a white solid. ¹H NMR (400 MHz, cd₃od) δ8.77 (s, 1H), 8.06 (d, J=2.8 Hz, 1H), 7.93 (s, 1H), 6.93 (d, J=6.5 Hz, 2H), 6.86 (d, J=2.9 Hz, 1H), 6.79 (t, J=9.1 Hz, 1H), 4.52 (d, J=15.7 Hz, 1H), 4.36 (d, J=15.7 Hz, 1H), 4.14 (t, J=6.7 Hz, 2H), 2.81 (dd, J=13.1, 7.1 Hz, 1H), 2.42-2.31 (m, 1H).

### Example 23. 2-(3-(3,5-difluorobenzylcarbamoyl)-3-hydroxy-2-oxopyrrolidin-1-yl)-6H-thieno[2,3-b]pyrro 1e-5-carboxamide (23)

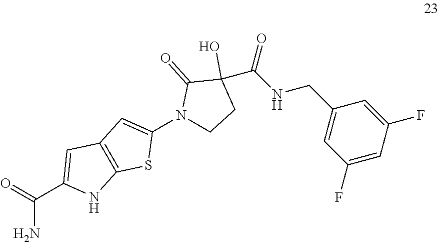 [SMILES: NC(=O)c1cc2cc(N3CCC(O)(C(=O)NCc4cc(F)cc(F)c4)C3=O)sc2[nH]1]

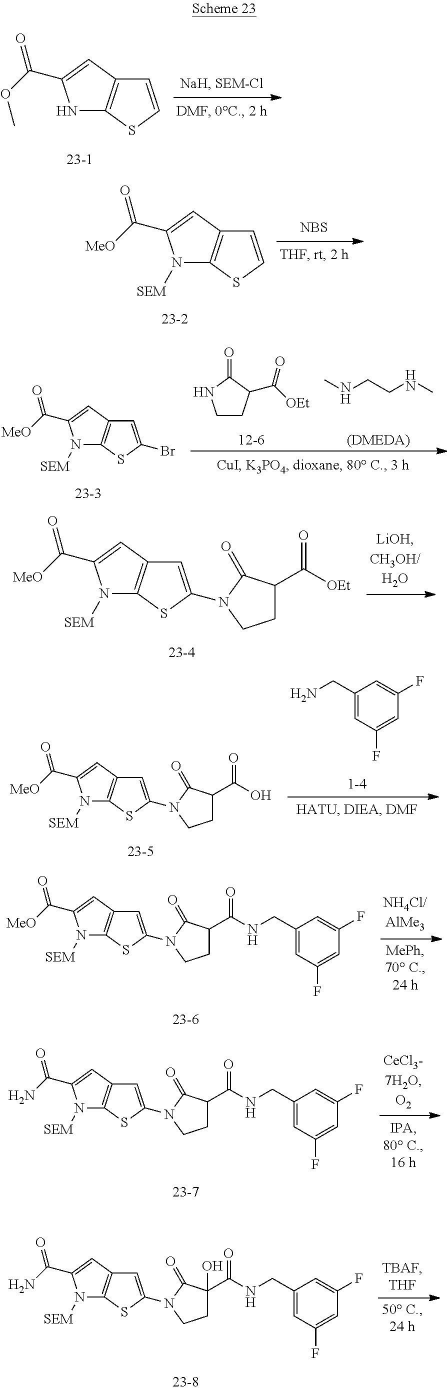

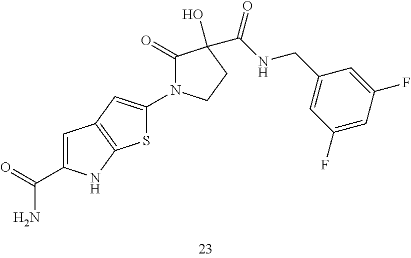 [SMILES: NC(=O)c1cc2cc(N3CCC(O)(C(=O)NCc4cc(F)cc(F)c4)C3=O)sc2[nH]1]

Step 1: To a solution of compound 23-1 (1 g, 5.5 mmol) in DMF (12 mL) was added 60% NaH (0.375 g, 9.4 mmol) at 0° C. The reaction mixture stirred for 0.5h at 0° C. And the mixture was added SEMCl (1.2 g, 7.2 mmol) at 0° C. Then the reaction was warmed to room temperature and stirred for 2h. The reaction was quenched with water. The mixture was extracted with ethyl acetate (3*20 mL). The combined organics were washed with water and brine, dried over sodium sulfate, filtered, and concentrated by rotary evaporation. The resulting residue was purified by column chromatography (10% EtOAc in Petrol) to give the compound 23-2 (1.55 g) as a yellow oil that partially solidified under vacuum. TLC (Petroleum ether: EtOAc, 10:1): R_{f} (23-2)=0.7.

Step 2: NBS (178 mg, 1.6 mmol) was added to a solution of compound 23-2 (500 mg, 1.6 mmol) in THF (10 mL) at 0° C., then the mixture was stirred at 30° C. for 2 hrs. LCMS showed the reaction convention was completely and the mixture was washed with H₂O (20 mL), extracted with EtOAc (20 mL*2). The combined organic layer was dried over anhydrous Na₂SO₄, concentrated in vacuo and purified by column chromatography (10% EtOAc in Petrol) to afford compound 23-3 (600 mg) as a yellow solid. TLC (Petroleum ether: EtOAc, 10:1): R_{f} (23-3)=0.7.

Step 3: To a solution of compound 23-3 (600 mg, 1.54 mmol) in dioxane (15 mL) was added ethyl 2-oxopyrrolidine-3-carboxylate 12-6 (290 mg, 1.84 mmol), CuI (146 mg, 0.77 mmol), DMEDA (54 mg, 0.616 mmol) and K₃PO₄ (980 mg, 4.62 mmol) under N₂. The reaction mixture was stirred for 3 h at 120° C. The reaction mixture was filtered by celite and wash with ethyl acetate, then organic layer was quenched with water and extracted with ethyl acetate (3*30 mL). The combined organics were washed with water and brine, dried over sodium sulfate, filtered, and concentrated by rotary evaporation. The resulting residue was purified by column chromatography (25% EtOAc in petroleum ether) to give the compound 23-4 (270 mg) as a yellow solid. TLC (Petroleum ether: EtOAc, 3:1): R_{f} (23-4)=0.5.

Step 4: LiGH H₂O (56 mg, 1.34 mmol) was added to a solution of Compound 23-4 (270 mg, 0.54 mmol) in MeOH/H₂O/THF (2 ml/2 ml/1 mL) under nitrogen atmosphere. The reaction mixture was stirred for 2h at room temperature. The combined organics were concentrated by rotary evaporation to give the compound 23-5 (250 mg) as a yellow solid. TLC (CH₂Cl₂: MeOH, 10:1): R_{f} (23-5)=0.2.

Step 5: To a solution of Compound 23-5 (250 mg, 0.54 mmol) in DMF (5 mL) was added compound 1-4 (86 mg), HATU (246 mg) and DIEA (210 mg). The reaction mixture was stirred at room temperature for 3h. The reaction was quenched with water. The mixture was extracted with ethyl acetate (3*20 mL). The combined organics were washed with water and brine, dried over sodium sulfate and concentrated by rotary evaporation. The resulting residue was purified by column chromatography (50% EtOAc in petroleum ether) to give the compound 23-6 (150 mg) as a white solid. TLC (Petroleum ether: EtOAc, 1:1): R_{f} (23-6)=0.3.

Step 5: AlMe₃ (1 M in hexane, 0.8 mL) was added to a solution of compound 23-6 (150 mg, 0.27 mmol) and NH₄Cl (200 mg, 2.7 mmol) in toluene (5 mL) and the mixture was stirred at 50° C. for 18 hrs, TLC showed 50% convention and longing time would not improve convention. The mixture was quenched with water. The mixture was extracted with ethyl acetate (3*10 mL). The combined organics were washed with water, dried over sodium sulfate and concentrated in vacuo. The resulting residue purified by prep-TLC (10% MeOH in CH₂Cl₂) to afford compound 23-7 (70 mg) as a yellow oil. TLC (CH₂Cl₂: MeOH, 10:1): R_{f} (23-7)=0.7.

Step 6: To a solution of compound 23-7 (70 mg) in IPA (2 mL) was added CeCl₃*7H₂O (110 mg, 0.3 mmol) under O₂. The reaction mixture was stirred at 70° C. for 12h. The mixture was quenched with water. The mixture was extracted with ethyl acetate (3* 10 mL). The combined organics were washed with water, dried over sodium sulfate and concentrated in vacuo. The resulting residue purified by prep-TLC (10% CH₃OH in CH₂Cl₂) to give 23-8 (40 mg) as a yellow oil. TLC (CH₂Cl₂: MeOH, 10:1): R_{f} (23-8)=0.5.

Step 7: TBAF (1 M in THF, 2 mL) was added to compound 23-8 (40 mg) and the mixture was stirred at 50° C. for 24h. The mixture was concentrated in vacuo and purified by prep-TLC (10% CH₃OH in CH₂Cl₂), further purified by prep-HPLC to afford compound 23 (7 mg) as a white solid. ¹H NMR (400 MHz, CD₃OD) δ8.82 (s, 1H), 6.88-6.96 (d, J=10.4 Hz, 3H), 6.79 (s, 1H), 6.66 (s, 1H), 4.42 (dd, J=15.6, 6.0 Hz, 2H), 4.06-3.88 (m, 2H), 2.75-2.80 (m, 1H), 2.24-2.33 (m, 1H). LCMS: m/z 435.25 [M+1], 433.20 [M-1];

### Example 24. Enzymatic activity assay of Methionine aminopeptidase 2

The MetAP-2 activity was determined by an enzyme coupled assay using the tripeptide Met-Ala-Ser (MAS) as substrate and recombinant human MetAP-2 (His-Tev-MetAP-2, prepared in-house). The released methionine is converted by L-amino acid oxidase (AAO) to Met ox and hydrogen peroxide is released. In a second step horse radish peroxidase catalyses the oxidation of the leuko dye dianisidine to dianisidine ox with hydrogen peroxide as co-substrate. The produced dianisidine ox was detected photometrically as increase in absorbance at 450 nm. MetAP-2 activity was determined in a kinetic measurement mode. The release of one molecule methionine corresponds to the production of one molecule dianisidine ox. The MetAP-2 enzymatic activity is directly corresponding to the increase in absorbance per time.

The IC₅₀ for the Methionine aminopeptidase 2 assay is shown in Table 4.

TABLE 4

| compound | MeAP2 IC50 |
|---|---|
| 1-7 | A |
| 1 | A |
| 1a | B |
| 2-8 | |
| 2 | A |
| 2a | B |
| 3-3 | |
| 3 | A |
| 3a | B |
| 4-5 | |
| 4 | A |
| 4a | A |
| 5-7 | A |
| 5 | A |
| 5a | B |
| 6-8 | A |
| 6 | A |
| 6a | C |
| 7-6 | |
| 7 | A |
| 7a | C |
| 8-5 | |
| 8 | A |
| 8a | B |
| 9-3 | |
| 9 | A |
| 9a | A |
| 10-5 | |
| 10 | A |
| 10a | B |
| 11 | A |
| 12 | A |
| 13 | A |
| 13a | B |
| 14 | A |
| 15 | A |
| 16 | A |
| 17 | A |
| 18 | A |
| 19 | A |
| 20 | A |
| 21 | |
| 22 | |
| 23 | A |

A: <1 μM;
B: 1-10 μM.
C: >10 μM

### Pharmaceutical Preparations

(A) Injection vials: A solution of 100 g of an active ingredient according to the invention and 5 g of disodium hydrogen phosphate in 31 of bidistilled water is adjusted to pH 6.5 using 2 N hydrochloric acid, sterile filtered, transferred into injection vials, is lyophilized under sterile conditions and is sealed under sterile conditions. Each injection vial contains 5 mg of active ingredient.
(B) Suppositories: A mixture of 20 g of an active ingredient according to the invention is melted with 100 g of soy lecithin and 1400 g of cocoa butter, is poured into moulds and is allowed to cool. Each suppository contains 20 mg of active ingredient.
(C) Solution: A solution is prepared from 1 g of an active ingredient according to the invention, 9.38 g of NaH₂PO₄·2 H₂O, 28.48 g of Na₂HIPO₄·12 H₂O and 0.1 g of benzalkonium chloride in 940 ml of bidistilled water. The pH is adjusted to 6.8, and the solution is made up to 11 and sterilized by irradiation. This solution could be used in the form of eye drops.
(D) Ointment: 500 mg of an active ingredient according to the invention is mixed with 99.5 g of Vaseline under aseptic conditions.
(E) Tablets: A mixture of 1 kg of an active ingredient according to the invention, 4 kg of lactose, 1.2 kg of potato starch, 0.2 kg of talc and 0.1 kg of magnesium stearate is pressed to give tablets in a conventional manner in such a way that each tablet contains 10 mg of active ingredient.
(F) Coated tablets: Tablets are pressed analogously to Example E and subsequently are coated in a conventional manner with a coating of sucrose, potato starch, talc, tragacanth and dye.
(G) Capsules: 2 kg of an active ingredient according to the invention are introduced into hard gelatin capsules in a conventional manner in such a way that each capsule contains 20 mg of the active ingredient.
(H) Ampoules: A solution of 1 kg of an active ingredient according to the invention in 60 1 of bidistilled water is sterile filtered, transferred into ampoules, is lyophilized under sterile conditions and is sealed under sterile conditions. Each ampoule contains 10 mg of active ingredient.
(I) Inhalation spray: 14 g of an active ingredient according to the invention are dissolved in 10 1 of isotonic NaCl solution, and the solution is transferred into commercially available spray containers with a pump mechanism. The solution could be sprayed into the mouth or nose. One spray shot (about 0.1 ml) corresponds to a dose of about 0.14 mg.

While a number of embodiments of this invention are described herein, it is apparent that the basic examples may be altered to provide other embodiments that utilize the compounds and methods of this invention. Therefore, it will be appreciated that the scope of this invention is to be defined by the appended claims rather than by the specific embodiments that have been represented by way of example.

Applicant's disclosure is described herein in preferred embodiments with reference to the Figures, in which like numbers represent the same or similar elements. Reference throughout this specification to “one embodiment,” “an embodiment,” or similar language means that a particular feature, structure, or characteristic described in connection with the embodiment is included in at least one embodiment of the present invention. Thus, appearances of the phrases “in one embodiment,” “in an embodiment,” and similar language throughout this specification may, but do not necessarily, all refer to the same embodiment.

The described features, structures, or characteristics of Applicant's disclosure may be combined in any suitable manner in one or more embodiments. In the description, herein, numerous specific details are recited to provide a thorough understanding of embodiments of the invention. One skilled in the relevant art will recognize, however, that Applicant's composition and/or method may be practiced without one or more of the specific details, or with other methods, components, materials, and so forth. In other instances, well-known structures, materials, or operations are not shown or described in detail to avoid obscuring aspects of the disclosure.

In this specification and the appended claims, the singular forms “a,” “an,” and “the” include plural reference, unless the context clearly dictates otherwise.

Unless defined otherwise, all technical and scientific terms used herein have the same meaning as commonly understood by one of ordinary skill in the art. Although any methods and materials similar or equivalent to those described herein can also be used in the practice or testing of the present disclosure, the preferred methods and materials are now described. Methods recited herein may be carried out in any order that is logically possible, in addition to a particular order disclosed.

### INCORPORATION BY REFERENCE

References and citations to other documents, such as patents, patent applications, patent publications, journals, books, papers, web contents, have been made in this disclosure. All such documents are hereby incorporated herein by reference in their entirety for all purposes. Any material, or portion thereof, that is said to be incorporated by reference herein, but which conflicts with existing definitions, statements, or other disclosure material explicitly set forth herein is only incorporated to the extent that no conflict arises between that incorporated material and the present disclosure material. In the event of a conflict, the conflict is to be resolved in favor of the present disclosure as the preferred disclosure.

### EQUIVALENTS

The representative examples are intended to help illustrate the invention, and are not intended to, nor should they be construed to, limit the scope of the invention. Indeed, various modifications of the invention and many further embodiments thereof, in addition to those shown and described herein, will become apparent to those skilled in the art from the full contents of this document, including the examples and the references to the scientific and patent literature included herein. The examples contain important additional information, exemplification and guidance that can be adapted to the practice of this invention in its various embodiments and equivalents thereof.

1-20. (canceled)

21. A compound having the following structural formula (III^{A}): 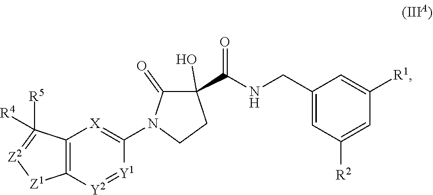
wherein
each of R¹ and R² is independently selected from F, Cl, Br, and I;
X is N or CH;
each of Yⁱ and Y² is independently N or CH, provided that if one of Y¹ and Y² is N, the other is CH;
Z¹ is NH;
Z² is C═O, S(═O)₂, or CR′R″ wherein R′ and R″ is independently H or a C₁₋₃ alkyl;
-- is absent; and
R⁴ and R⁵, together with the carbon atom to which they are attached, form a 3- to 6-membered aliphatic ring,
or a pharmaceutically acceptable form or an isotope derivative thereof.

22. (canceled)

23. The compound of claim 21, wherein Z¹ is NH and Z² is CH₂;

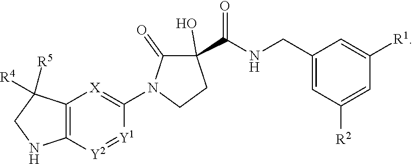

24. The compound of claim 21, wherein Z¹ is NH and Z² is C═O

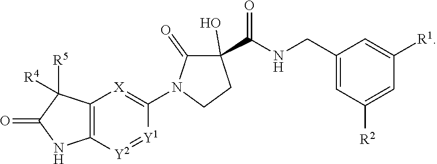

25. The compound of claim 21, wherein Z¹ is NH and Z² is S(═O)₂

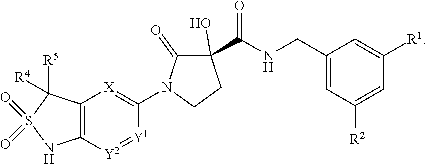

26. The compound of claim 24, wherein each of X, Y¹ and Y² is CH.

27. The compound of claim 24, wherein one of X, Y¹ and Y² is not CH.

28. The compound of claim 24, wherein X is CH, Y¹ is CH, and Y² is N.

29. The compound of claim 24, wherein X is N, Y¹ is CH, and Y² is N.

30. The compound of claim 24, wherein X is CH, Y¹ is N, and Y² is CH.

33-64. (canceled)

65. The compound of claim 21, selected from:

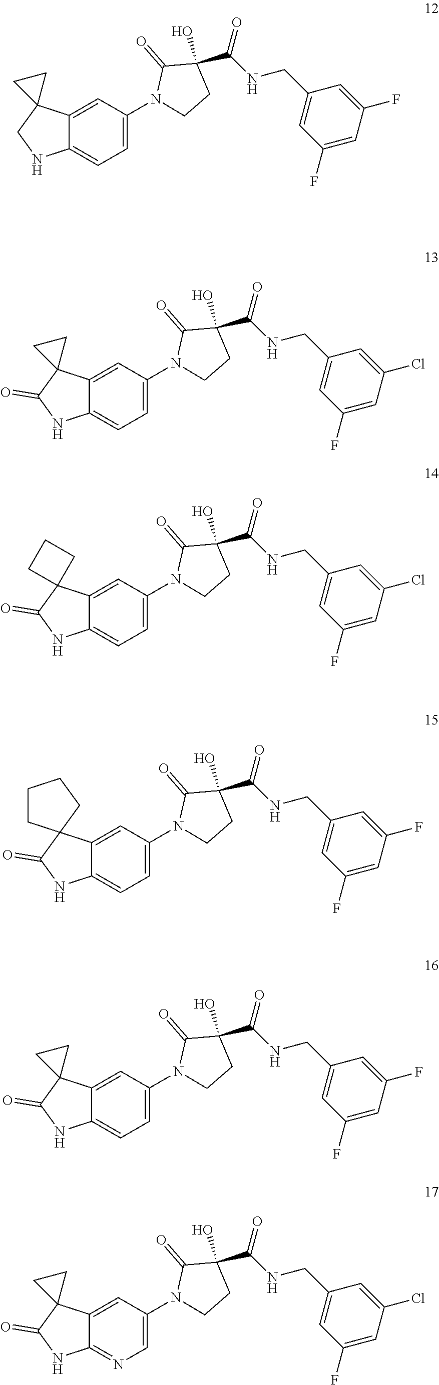 [SMILES: O=C(NCc1cc(F)cc(F)c1)[C@@]1(O)CCN(c2ccc3c(c2)C2(CC2)CN3)C1=O ; O=C1Nc2ccc(N3CC[C@](O)(C(=O)NCc4cc(F)cc(Cl)c4)C3=O)cc2C12CC2 ; O=C1Nc2ccc(N3CC[C@](O)(C(=O)NCc4cc(F)cc(Cl)c4)C3=O)cc2C12CCC2 ; O=C1Nc2ccc(N3CC[C@](O)(C(=O)NCc4cc(F)cc(F)c4)C3=O)cc2C12CCCC2 ; O=C1Nc2ccc(N3CC[C@](O)(C(=O)NCc4cc(F)cc(F)c4)C3=O)cc2C12CC2 ; O=C1Nc2ncc(N3CC[C@](O)(C(=O)NCc4cc(F)cc(Cl)c4)C3=O)cc2C12CC2]

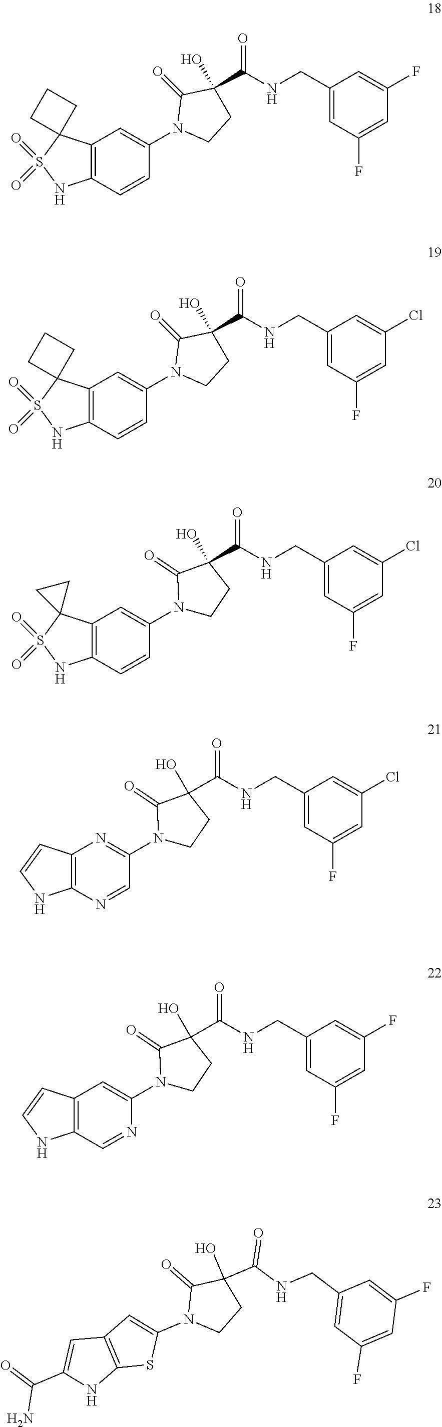 [SMILES: O=C(NCc1cc(F)cc(F)c1)[C@@]1(O)CCN(c2ccc3c(c2)C2(CCC2)S(=O)(=O)N3)C1=O ; O=C(NCc1cc(F)cc(Cl)c1)[C@@]1(O)CCN(c2ccc3c(c2)C2(CCC2)S(=O)(=O)N3)C1=O ; O=C(NCc1cc(F)cc(Cl)c1)[C@@]1(O)CCN(c2ccc3c(c2)C2(CC2)S(=O)(=O)N3)C1=O ; O=C(NCc1cc(F)cc(Cl)c1)C1(O)CCN(c2cnc3[nH]ccc3n2)C1=O ; O=C(NCc1cc(F)cc(F)c1)C1(O)CCN(c2cc3cc[nH]c3cn2)C1=O ; NC(=O)c1cc2cc(N3CCC(O)(C(=O)NCc4cc(F)cc(F)c4)C3=O)sc2[nH]1]

66. A pharmaceutical composition comprising a compound of claim 21 and a pharmaceutically acceptable excipient, carrier, or diluent.

67. The compound of claim 65, having the structural formula:

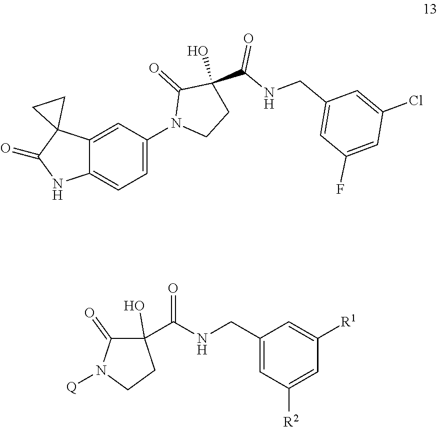

68-75. (canceled)

76. A unit dosage form comprising a pharmaceutical composition according to claim 66.

77. A method for treating, reducing, or preventing a disease or condition, comprising administering to a subject in need thereof a pharmaceutical composition comprising a compound of claim 21 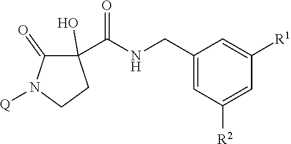
and a pharmaceutically acceptable excipient, carrier, or diluent, wherein the disease or condition is selected from cancer, obesity, diabetes, rheumatoid arthritis and psoriasis, or a related disease or condition thereof.

78-95. (canceled)
